SEQUENCE LISTING (34 sequences)

SEQ ID NO: 1 - protein, 2 aa, Artificial Sequence
  Peptide linker
  misc_feature (1)..(2): wherein the sequence can be repeated from an integer from 1-50
  misc_feature (1)..(1): wherein the amino acid can be present 0 or 1 time
  misc_feature (1)..(1): wherein the amino acid can be repeated 1-4 times
GS

SEQ ID NO: 2 - protein, 4 aa, Artificial Sequence
  peptide linker
GGGG

SEQ ID NO: 3 - protein, 2 aa, Artificial Sequence
  peptide linker
  MISC_FEATURE (1)..(2): wherein the sequence can be repeated from an integer between 1 and 100
GA

SEQ ID NO: 4 - protein, 3 aa, Artificial Sequence
  Peptide linker
  MISC_FEATURE (1)..(3): wherein the sequence can be repeated from an integer between 1 and 100
GGS

SEQ ID NO: 5 - protein, 4 aa, Artificial Sequence
  Peptide linker
  MISC_FEATURE (1)..(4): wherein the sequence can be repeated from an integer from 1-100
GGGS

SEQ ID NO: 6 - protein, 8 aa, Artificial Sequence
  Peptide linker
  MISC_FEATURE (1)..(3): wherein the sequence can be repeated from an integer from 1-100
  MISC_FEATURE (1)..(5): wherein the sequence can be repeated from an integer from 1-100
GGSGGGGS

SEQ ID NO: 7 - protein, 7 aa, Artificial Sequence
  Peptide linker
SGGSGGS

SEQ ID NO: 8 - protein, 15 aa, Artificial Sequence
  Peptide linker
GGSGGSGGSGGSGGG

SEQ ID NO: 9 - protein, 16 aa, Artificial Sequence
  Peptide linker
GGSGGSGGGGSGGGGS

SEQ ID NO: 10 - protein, 18 aa, Artificial Sequence
  Peptide linker
GGSGGSGGSGGSGGSGGS

SEQ ID NO: 11 - protein, 15 aa, Artificial Sequence
  Peptide linker
GGGGSGGGGSGGGGS

SEQ ID NO: 12 - protein, 4 aa, Artificial Sequence
  Peptide linker
  MISC_FEATURE (1)..(4): wherein the sequence can be repeated from an integer from 1-100
GGGG

SEQ ID NO: 13 - DNA, 32 bp, Artificial Sequence
  HA-5′-UTR
agcaaaagcaggggaaaataaaagcaacaaaa

SEQ ID NO: 14 - DNA, 44 bp, Artificial Sequence
  HA-3′-UTR
cattaggatttcagaagcatgagaaaaacacccttgtttctact

SEQ ID NO: 15 - DNA, 287 bp, Artificial Sequence
  2hBgUTR
ctcgagagctcgctttcttgctgtccaatttctattaaaggttcctttgttccctaagtccaactactaaactgggggatattatgaagggccttgagcatctggattctgcctaataaaaaacatttattttcattgctgcgtcgagagctcgctttcttgctgtccaatttctattaaaggttcctttgttccctaagtccaactactaaactgggggatattatgaagggccttgagcatctggattctgcctaataaaaaacatttattttcattgctgcgtc

SEQ ID NO: 16 - DNA, 4302 bp, Artificial Sequence
  Codon-optimized coronavirus spike nucleotide sequence
atgttcgtgttccttgtgctgctgcctctggtgtccagccagtgcgtgaacctgacaacaagaacccagctgccacctgcctatacaaacagcttcaccaggggagtctactaccctgacaaggtgttcagatccagcgtcctgcattctacccaggacctgttcctgcctttcttcagcaacgtcacatggtttcacgccatccacgtgtctggaaccaacggaacaaagagattcgataaccccgtactgcctttcaatgacggcgtttactttgcctctacggagaagtcaaacattattcggggctggatcttcggcacgaccctggacagcaagacccagtctctgctgatagtgaacaacgccacaaacgtggtcatcaaggtgtgtgaattccagttctgcaacgatcctttcctgggcgtgtattatcacaagaacaacaagagctggatggaaagcgaattccgcgtttacatgttcgtgttccttgtgctgctgcctctggtgtccagccagtgcgtgaacctgacaacaagaacccagctgccacctgcctatacaaacagcttcaccaggggagtctactaccctgacaaggtgttcagatccagcgtcctgcattctacccaggacctgttcctgcctttcttcagcaacgtcacatggtttcacgccatccacgtgtctggaaccaacggaacaaagagattcgataaccccgtactgcctttcaatgacggcgtttactttgcctctacggagaagtcaaacattattcggggctggatcttcggcacgaccctggacagcaagacccagtctctgctgatagtgaacaacgccacaaacgtggtcatcaaggtgtgtgaattccagttctgcaacgatcctttcctgggcgtgtattatcacaagaacaacaagagctggatggaaagcgaattccgcgtttactctagcgccaacaactgcaccttcgagtacgtgagccagccattcctgatggacctcgaaggcaagcagggcaactttaagaatctcagagaattcgtgttcaagaatatcgacggctacttcaaaatctatagcaagcacaccccaatcaacctggtcagagatctgcctcagggcttcagcgccctggaacctctggtggatctgcctatcggaatcaacatcaccaggttccagaccctgctggccctgcatcggagctacctgacccccggagatagcagcagcggctggaccgccggcgccgccgcttactacgtgggctacctgcaacctagaaccttcctgctcaaatacaacgagaatggaacaatcacagacgccgtggactgcgccctcgaccctctgtccgagacaaaatgtacactgaagtcttttacagtggaaaagggcatataccagacaagcaattttcgggtccagcctaccgagagcatcgtgagatttcccaacattaccaatctgtgtcctttcggcgaggtgttcaacgccactcggtttgcctctgtgtacgcctggaacagaaaacggatcagcaactgcgtggccgactactctgtcctttataacagcgcctcctttagcaccttcaagtgctacggcgtgtcacccaccaagctgaacgacctgtgcttcacaaacgtgtacgccgacagcttcgtgatccggggcgatgaggttcgacagatcgcccctggacagaccggcaagatcgctgactacaactataagctgcccgatgacttcaccggctgcgtgatcgcctggaatagcaataacctggacagcaaagtgggcggaaactacaactacctgtaccgcctctttagaaagagcaatctgaagcctttcgaaagagatatctctacagagatctaccaggccggctccactccttgtaatggcgtcgagggctttaactgttacttccctctgcagtcctatggcttccagcctaccaacggcgtggggtaccagccctacagagtggtggtgctgagctttgagctgctgcacgcacctgccaccgtgtgcggtcctaagaaaagcaccaacctggtgaagaacaagtgcgtgaattttaacttcaacggactgaccggcaccggcgtgttgaccgaatctaacaagaagttcctgcccttccagcagttcggcagagacatagctgacaccaccgatgccgtgcgggaccctcagaccctggaaatcctggacattaccccatgcagcttcggcggcgtgtctgtcatcacccctggcaccaacacctccaatcaagtggctgtcctgtaccaagatgtgaactgcacagaagtgcctgtggctatccacgccgaccagctgacgcccacctggcgggtgtacagcacgggcagcaacgtgttccagacccgggctggctgcctgatcggagccgagcacgtgaacaacagctacgaatgcgacatcccgatcggcgccggcatctgcgccagctaccaaacacagaccaatagcccaagacgggccagatccgtggctagccaaagcatcatcgcctacacaatgagcctgggcgctgagaactccgttgcctatagcaacaacagcatcgccatccccacaaactttaccatttctgtgaccacagaaatcctgcctgtgtctatgaccaagacatctgtggactgcaccatgtacatctgcggagacagcaccgagtgctccaacctgctgctgcagtacggctccttctgcacccaacttaatagagccctcaccggtatcgctgttgagcaggacaagaacactcaggaggtgttcgctcaggtgaagcaaatctacaagacccctcctatcaaggacttcggaggctttaacttcagccaaatcctgcctgacccatctaaacctagcaagagaagcttcatcgaggacctgctgttcaacaaggtgaccctggccgatgccggcttcatcaaacagtacggcgactgcctgggcgacatcgcggcccgtgatctcatctgcgctcaaaagttcaacggcctgaccgtgctgccccccctactgacagatgaaatgatcgcccagtacacctctgccctgctggctggcacaatcacctccggctggacatttggcgctggagccgccctgcagatccctttcgccatgcagatggcctacagattcaacggcatcggcgtgacacagaacgtgctgtacgagaaccagaagctgatcgccaaccagtttaattctgctatcggcaaaatccaggactccctgtcctccacagcctccgccctgggcaaactgcaggacgtggtgaaccaaaatgcccaggccttgaacaccctggtgaagcagctgagcagtaactttggagccatcagctctgtgctgaatgatatcctgtctcggctggataaggtcgaggccgaggtgcagatcgatagactgattaccggtagactgcagagcctgcagacatacgtgacccagcagctgatccgggccgccgaaattagagcctctgcaaatctggctgctaccaagatgagcgagtgtgtgctgggccagagcaagagagtcgacttttgtggcaaaggctaccacctgatgagcttccctcagtctgccccccacggcgtggtcttcctgcacgtgacgtacgtgcccgcccaggagaagaacttcacaaccgcccctgccatctgccatgacggcaaggcccacttccccagagaaggcgtgttcgttagcaacggcacccactggttcgtgacacagagaaacttctacgagccccagatcattacaacggacaataccttcgtgtctggcaactgtgacgtcgtgatcggcatcgttaacaacacagtgtacgaccccctgcagcctgagctggattctttcaaagaggaactggacaagtacttcaagaaccacaccagcccagatgtggatctgggcgacattagcggaatcaacgcaagcgtggtgaatatccagaaggaaatcgaccggctgaacgaggtggcaaagaaccttaatgaaagcttgattgacctgcaagagctgggcaagtacgagcagtatatcaagtggccttggtacatctggctaggcttcattgccggactgatcgccatcgtgatggtgacaatcatgctgtgctgcatgacatcttgttgtagctgcctgaaaggctgttgtagctgtggaagctgctgcaagttcgacgaagatgattcagagcctgtgcttaagggcgtgaagctgcactacacatga

SEQ ID NO: 17 - DNA, 3822 bp, Artificial Sequence
  Wild-type coronavirus spike nucleotide sequence
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 18 - protein, 1273 aa, Artificial Sequence
  Wild-type coronavirus spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 19 - protein, 13 aa, Artificial Sequence
  Spike protein - signal peptide
MFVFLVLLPLVSS

SEQ ID NO: 20 - protein, 1199 aa, Artificial Sequence
  Spike protein - extracellular domain
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKW

SEQ ID NO: 21 - protein, 24 aa, Artificial Sequence
  Spike protein - transmembrane domain
PWYIWLGFIAGLIAIVMVTIMLCC

SEQ ID NO: 22 - protein, 37 aa, Artificial Sequence
  Spike protein - cytoplasmic domain
MTSCCSCLKGCCSCGSCCKFDEDDSEPVLKGVKLHYT

SEQ ID NO: 23 - protein, 672 aa, Artificial Sequence
  Spike protein - S1 Subunit
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 24 - protein, 588 aa, Artificial Sequence
  Spike protein - S2 Subunit
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1260 aa, Artificial Sequence
  Coronavirus mature spike protein
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - protein, 1236 aa, Artificial Sequence
  Mature spike protein without transmembrane domain
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWMTSCCSCLKGCCSCGSCCKFDEDDSEPVLKGVKLHYT

SEQ ID NO: 27 - protein, 1249 aa, Artificial Sequence
  Spike protein without transmembrane domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWMTSCCSCLKGCCSCGSCCKFDEDDSEPVLKGVKLHYT

SEQ ID NO: 28 - protein, 1223 aa, Artificial Sequence
  Mature spike protein without cytoplasmic domain
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCC

SEQ ID NO: 29 - protein, 1236 aa, Artificial Sequence
  Spike protein without cytoplasmic domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCC

SEQ ID NO: 30 - protein, 1199 aa, Artificial Sequence; identical to SEQ ID NO: 20
  Mature spike protein without TM and cytoplasmic domain

SEQ ID NO: 31 - protein, 1212 aa, Artificial Sequence
  Spike protein without TM and cytoplasmic domain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKW

SEQ ID NO: 32 - protein, 1 aa, Artificial Sequence
  Peptide linker
  MISC_FEATURE (1)..(1): where the amino acid can be repeated from an integer from 1-100
G

SEQ ID NO: 33 - DNA, 73 bp, Artificial Sequence
  hBg-5′-UTR
cagggcagagccatctattgcttacatttgcttctgacacaactgtgttcactagcaacctcaaacagacacc

SEQ ID NO: 34 - DNA, 131 bp, Artificial Sequence
  hBg-3′-UTR
gctcgctttcttgctgtccaatttctattaaaggttcctttgttccctaagtccaactactaaactgggggatattatgaagggccttgagcatctggattctgcctaataaaaaacatttattttcattg

(19) United States
(12) Patent Application Publication
(10) Pub. No.: US 2024/0209035 A1
(43) Pub. Date: Jun. 27, 2024

(54) POLYNUCLEOTIDES CAPABLE OF ENHANCED PROTEIN EXPRESSION AND USES THEREOF

(71) Applicant: BIORCHESTRA CO., LTD., Daejeon (KR)

(72) Inventors: Jin-Hyeob RYU, Daejeon (KR); Sang Moo LEE, Daejeon (KR); Eun Ha KIM, Daejeon (KR); Rak Kyun SEONG, Daejeon (KR); Hyun Su MIN, Daejeon (KR); Yu Na LIM, Daejeon (KR)

(73) Assignee: BIORCHESTRA CO., LTD., Daejeon (KR)

(21) Appl. No.: 18/555,304

(22) PCT Filed: Apr. 15, 2022
(86) PCT No.: PCT/IB2022/053578

§ 371 Date: Oct. 13, 2023

Related U.S. Application Data
(60) Provisional application No. 63/175,459, filed on Apr. 15, 2021.

(51) Int. Cl. C07K 14/005; A61K 39/00; A61K 39/145; A61K 39/215; A61P 31/16; C07K 14/705

(52) U.S. Cl. CPC C07K 14/005; A61K 39/001186; A61K 39/001188; A61K 39/145; A61K 39/215; A61P 31/16; C07K 14/705; A61K 2039/53; A61K 2039/55511; A61K 2039/6093; C12N 2760/16122; C12N 2760/16134; C12N 2770/20022; C12N 2770/20034; C12N 2830/50

(57) ABSTRACT

The present disclosure is directed to polynucleotides comprising an ORF encoding a protein of interest and UTRs (e.g., 5′-UTR and 3′-UTR), wherein the UTRs are heterologous to the encoded protein and capable of increasing the expression of the encoded protein, compared to a corresponding polynucleotide without the UTRs. The present disclosure is also directed to the use of such polynucleotides to treat various diseases and disorders.

### FIELD OF THE DISCLOSURE

This PCT application claims the priority benefit of U.S. Provisional Application No. 63/175,459, filed on Apr. 15, 2021, which is herein incorporated by reference in its entirety.

### REFERENCE TO SEQUENCE LISTING SUBMITTED ELECTRONICALLY VIA EFS-WEB

The content of the electronically submitted sequence listing (Name: 4366_028PC01_Seqlisting_ST25.txt, Size: 124,555 bytes; and Date of Creation: Apr. 15, 2022) submitted in this application is incorporated herein by reference in its entirety.

### FIELD OF THE DISCLOSURE

The present disclosure provides modified polynucleotides that are capable of inducing an enhanced expression of the encoded protein, and the uses of such polynucleotides to treat various diseases and disorders.

### BACKGROUND OF THE DISCLOSURE

Vaccines have had profound effect on world health. For instance, small pox has been eradicated, and polio is near elimination. Nonetheless, as evidenced by the recent coronavirus pandemic, there remains many human pathogens that have yet to be successfully contained. Moreover, conventional vaccination strategies generally involve the administration of either “live” (attenuated) or “dead” vaccines. Such vaccines often have undesirable side effects and risks (e.g., live attenuated vaccines can revert to pathogenic organism) or have limited efficacy.

More recently, there has been much interest in the use of polynucleotides as a vaccine platform (e.g., DNA and mRNA vaccines). Compared to conventional vaccines, polynucleotide-based vaccines are generally safer, easier to manufacture, and are capable of inducing wider range of immune responses. However, generally, such vaccines have limited efficacy, particularly in humans. See Hobernik et al., Int J Mol Sci 19(11): 3605 (November 2018). As a result, to date, there are still no DNA vaccines that have been approved for human use. And, only due to the recent pandemic, three coronavirus-specific mRNA vaccines have been approved. Accordingly, there remains a need for new polynucleotide-based vaccines with greater potency for human use.

### BRIEF SUMMARY OF THE DISCLOSURE

Provided herein is an isolated polynucleotide comprising an open reading frame (ORF) and (i) a 5′-untranslated region element (5′-UTR) of an influenza hemagglutinin (HA) protein, (ii) a 3′-untranslated region element (3′-UTR) of an influenza hemagglutinin (HA) protein, or both (i) and (ii); wherein the ORF encodes a protein that is heterologous to the 5′-UTR, 3′-UTR, or both 5′-UTR and 3′-UTR. In some aspects, the isolated polynucleotide comprises both the 5′-UTR and the 3′-UTR.

In some aspects, the 5′-UTR comprises the nucleic acid sequence set forth in SEQ ID NO: 13 (AGCAAAAGCAGGGGAAAATAAAAGCAACAAAA). In some aspects, the 5′-UTR consists of the nucleic acid sequence set forth in SEQ ID NO: 13 (AGCAAAAGCAGGGGAAAATAAAAGCAACAAAA). In some aspects, the 3′-UTR comprises the nucleic acid sequence set forth in SEQ ID NO: 14 (CATTAGGATTTCAGAAGCATGAGAAAAACACCCTTGTTTCTACT). In some aspects, the 3′-UTR consists of the nucleic acid sequence set forth in SEQ ID NO: 14 (CATTAGGATTTCAGAAGCATGAGAAAAACACCCTTGTTTCTACT).

In some aspects, the 5′-UTR, the 3′-UTR, or both the 5′-UTR and the 3′-UTR are capable of increasing the expression of the heterologous protein encoded by the ORF when transfected in a cell, compared to a corresponding expression in a cell transfected with a reference polynucleotide that does not comprise both the 5′-UTR and the 3′-UTR.

In some aspects, an isolated polynucleotide of the present disclosure further comprises a 5′-cap, a poly(A) tail, at least one translation enhancer element (TEE), a translation initiation sequence, at least one microRNA binding site or seed thereof, a 3′ tailing region of linked nucleosides, an AU rich element (ARE), a post transcription control modulator, or combinations thereof.

In some aspects, the 5′-cap comprises m₂^{7,2′-O}GppspGRNA, m⁷GpppG, m⁷GPPPPm⁷G, m₂^{(7,3′-O)}GPPPG, m₂^{(7,2′-O)}GppspG(D1), m₂^{(7,2′-O)}GppspG(D2), m₂^{7,3′-O}Gppp(m₁^{2′-O})ApG, (m⁷G-3′ mppp-G; which may equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, or combinations thereof. In some aspects, the 3′ tailing region of linked nucleosides comprises a poly-A tail, a polyA-G quartet, or a stem loop sequence.

In some aspects, an isolated polynucleotide of the present disclosure comprises at least one modified or non-naturally occurring nucleotide. In certain aspects, the least one modified or non-naturally occurring nucleotide comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, or combinations thereof.

In some aspects, the heterologous protein encoded by the ORF of an isolated polynucleotide described herein comprises a coronavirus protein. In some aspects, the coronavirus protein comprises a SARS-CoV-2 spike protein.

In some aspects, the heterologous protein encoded by the ORF of an isolated polynucleotide described herein comprises an influenza protein. In certain aspects, the influenza protein comprises a HA protein, a neuraminidase (NA) protein, a nucleoprotein (NP), a matrix 1 (M1) protein, a matrix 2 (M2) protein, a non-structural protein 1 (NS1), a non-structural protein 2 (NS2), a polymerase acidic (PA) protein, a polymerase basic 1 (PB1) protein, a PB1-F2 protein, a polymerase basic 2 (PB2) protein, or any combination thereof.

In some aspects, the heterologous protein encoded by the ORF comprises a tumor antigen. In some aspects, the tumor antigen comprises an alpha-fetoprotein (AFP), B-cell maturation antigen (BCMA), carcinoembryonic antigen (CEA), epithelial tumor antigen (ETA), mucin 1 (MUC1), Tn-MUC1, mucin 16 (MUC16), tyrosinase, melanoma-associated antigen (MAGE; e.g., MAGEA3), tumor protein p53 (p53), CD4, CD8, CD45, CD80, CD86, programmed death ligand 1 (PD-L1), programmed death ligand 2 (PD-L2), prostate-specific membrane antigen (PSMA), TAG-72, human epidermal growth factor receptor 2 (HER2), GD2, cMET, EGFR, mesothelin, VEGFR, alpha-folate receptor, CE7R, IL-3, cancer-testis antigen (e.g., New York esophageal squamous cell carcinoma 1 (NY-ESO-1), MART-1 gp100, ROR1, ROR2, glypican-2, glypican-3, TNF-related apoptosis-inducing ligand, or combinations thereof.

In some aspects, the heterologous protein encoded by the ORF of an isolated polynucleotide described herein comprises a protein associated with a genetic disorder. In certain aspects, the genetic disorder comprises a Hunter syndrome.

Also provided herein is an isolated polynucleotide comprising, from 5′ to 3′: (a) a 5′-untranslated region element (5′-UTR) of an influenza hemagglutinin (HA) protein, which comprises the nucleic acid sequence set forth in SEQ ID NO: 13 (AGCAAAAGCAGGGGAAAATAAAAGCAACAAAA); (b) an open reading frame (ORF);
and (c) a 3′-untranslated region element (3′-UTR) of an influenza hemagglutinin (HA) protein, which comprises the nucleic acid sequence set forth in SEQ ID NO: 14 (CATTAGGATTTCAGAAGCATGAGAAAAACACCCTTGTTTCTACT); wherein the ORF encodes a protein that is heterologous to both the 5′-UTR and the 3′-UTR.

Present disclosure further provides a vector comprising any of the isolated polynucleotides described herein. Also provided herein is a cell comprising any of the isolated polynucleotides or vectors described herein.

Provided herein is a pharmaceutical composition comprising (i) any of the isolated polynucleotides, vectors, or cells described herein; and (ii) a pharmaceutically acceptable excipient. Also provided herein is a kit comprising (i) any of the isolated polynucleotides, vectors, or cells described herein; and (ii) instructions for use.

Also provided herein is a method of treating a disease or disorder in a subject in need thereof, comprising administering to the subject any of the isolated polynucleotides described herein. In some aspects, the disease or disorder that can be treated comprises a viral infection, cancer, genetic disorder, or combinations thereof. In some aspects, the viral infection comprises a coronavirus infection, influenza virus infection, or both. In some aspects, the cancer comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or combinations thereof. In certain aspects, the genetic disorder comprises a Hunter syndrome.

Also provided herein is a method of increasing the expression of a protein, comprising contacting a cell with any of the isolated polynucleotides of the present disclosure. In some aspects, the contacting occurs in vivo. In some aspects, the contacting occurs ex vivo. In some aspects, the expression of the protein is increased by at least about 0.5-fold, about 1-fold, about 2-fold, about 3-fold, about 4-fold, about 5-fold, about 10-fold, about 20-fold, about 30-fold, about 40-fold, or about 50-fold, compared to the expression of the protein in a cell contacted with a reference polynucleotide that does not comprise both the 5′-UTR and the 3′-UTR.

In some aspects, the isolated polynucleotide provided herein is delivered, e.g., to a subject, in a delivery agent. In certain aspects, the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

In some aspects, the delivery agent comprises a cationic carrier unit which comprises:

[WP]-L1-[CC]-L2-[AM] (formula I)

or

[WP]-L1-[AM]-L2-[CC] (formula II),
wherein
WP is a water-soluble biopolymer moiety;
CC is a cationic carrier moiety;
AM is an adjuvant moiety; and,
L1 and L2 are independently optional linkers.

In some aspects, the cationic carrier unit and the isolated polynucleotide are capable of associating with each other to form a micelle when mixed together. In some aspects, the association is via a covalent bond. In some aspects, the association is via a non-covalent bond. In some aspects, the non-covalent bond comprises an ionic bond.

In some aspects, the water-soluble polymer of the cationic carrier unit comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”).

In some aspects, the water-soluble polymer comprises:

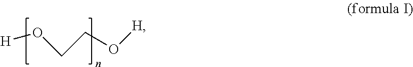

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In certain aspects, the n is about 80 to about 90, about 90 to about 100, about 100 to about 110, about 110 to about 120, about 120 to about 130, about 140 to about 150, or about 150 to about 160. In some aspects, the n is about 114.

In some aspects, the water-soluble polymer of the cationic carrier unit is linear, branched, or dendritic.

In some aspects, the cationic carrier moiety comprises one or more basic amino acids. In some aspects, the cationic carrier moiety comprises at least about three, at least about four, at least about five, at least about six, at least about seven, at least about eight, at least about nine, at least about ten, at least about 11, at least about 12, at least about 13, at least about 14, at last about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 basic amino acids. In some aspects, the cationic carrier moiety comprises about 60, about 70, about 80, about 90, or about 100 basic amino acids. In some aspects, the cationic carrier moiety comprises about 80 basic amino acids.

In some aspects, the basic amino acid comprises arginine, lysine, histidine, or any combination thereof. In some aspects, the cationic carrier moiety comprises about 80 lysine monomers.

In some aspects, the adjuvant moiety of the cationic carrier unit is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

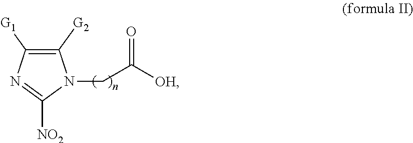

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof.

In some aspects, the adjuvant moiety comprises an amino acid. In some aspects, the adjuvant moiety comprises

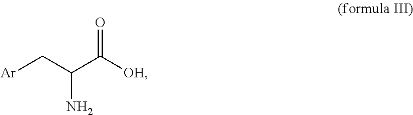

wherein Ar is

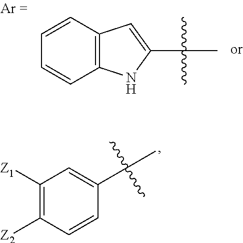

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

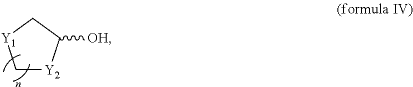

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

In some aspects, the vitamin is vitamin B3. In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, or at least about 50 vitamin B3. In some aspects, the adjuvant moiety comprises about 35 vitamin B3.

In some aspects, the delivery agent comprises a water-soluble biopolymer moiety with about 120 to about 130 PEG units, a cationic carrier moiety comprising a poly-lysine with about 80 lysines, and an adjuvant moiety with about 35 vitamin B3.

In some aspects, the delivery agent comprises a cationic carrier unit comprising:

[CC]-L1-[CM]-L2-[HM] (Schema I);

[CC]-L1-[HM]-L2-[CM] (Schema II);

[HM]-L1-[CM]-L2-[CC] (Schema III);

[HM]-L1-[CC]-L2-[CM] (Schema IV);

[CM]-L1-[CC]-L2-[HM] (Schema V); or

[CM]-L1-[HM]-L2-[CC] (Schema VI);
wherein
CC is a positively charged carrier moiety;
CM is a crosslinking moiety;
HM is a hydrophobic moiety; and,
L1 and L2 are independently optional linkers, and
wherein the number of HM is less than 40% relative to [CC] and [CM].

In some aspects, the number of HM is less than 39%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or about 1% relative to [CC] and [CM]. In some aspects, the cationic carrier unit is capable of interacting with any of the isolated polynucleotides described herein.

In some aspects, the cationic carrier moiety comprises at least about three, at least about four, at least about five, at least about six, at least about seven, at least about eight, at least about nine, at least about ten, at least about 11, at least about 12, at least about 13, at least about 14, at least 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 amino acids. In some aspects, the cationic carrier moiety comprises 80 amino acids.

In some aspects, the amino acids comprise a lysine.

In some aspects, the hydrophobic moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, or at least about 32 amino acids, each linked to a vitamin. In some aspects, the hydrophobic moiety comprises about two vitamin B3, about three vitamin B3, about four vitamin B3, about five vitamin B3, about six vitamin B3, about seven vitamin B3, about eight vitamin B3, about nine vitamin B3, about ten vitamin B3, about 11 vitamin B3, about 12 vitamin B3, about 13 vitamin B3, about 14 vitamin B3, about 15 vitamin B3, about 16 vitamin B3, about 17 vitamin B3, about 18 vitamin B3, about 19 vitamin B3, about 20 vitamin B3, about 21 vitamin B3, about 22 vitamin B3, about 23 vitamin B3, about 24 vitamin B3, about 25 vitamin B3, about 26 vitamin B3, about 27 vitamin B3, about 28 vitamin B3, about 29 vitamin B3, about vitamin B3, about 31 vitamin B3, about 32 vitamin B3, about 33 vitamin B3, about 34 vitamin B3, or about 35 vitamin B3.

In some aspects, a cationic carrier moiety of the delivery agent described herein comprises about 35 to about 45 lysines, the crosslinking moiety comprises about 5 to about 40 lysine-thiol, and the hydrophobic moiety comprises about 1 to about 50 lysine-vitamin B3. In certain aspects, the cationic carrier moiety comprises about 40 lysines, the crosslinking moiety comprises about 5 lysine-thiol, and the hydrophobic moiety comprises about 35 lysine-vitamin B3. In some aspects, the water-soluble biopolymer moiety comprises about 114 PEG units.

In some aspects, any of the isolated polynucleotides described herein is administered, e.g., to a subject, parenthetically, intramuscularly, subcutaneously, ophthalmic, intravenously, intraperitoneally, intradermally, intraorbitally, intranasally, intracerebrally, intracranially, intracerebroventricularly, intraspinally, intraventricular, intrathecally, intracistemally, intracapsularly, intratumorally, topically, or any combination thereof.

### BRIEF DESCRIPTION OF THE DRAWINGS/FIGURES

FIG. 1 provides a schematic of an exemplary polynucleotide described herein. As shown, the polynucleotide comprises (from 5′ to 3′): (i) a 5′-cap; (ii) HA-5′-UTR (SEQ ID NO: 13); (iii) an open reading frame encoding a protein of interest (“antigen-CDS”); (iv) HA-3′-UTR (SEQ ID NO: 14); and (v) a 3′-poly(A) tail.

FIG. 2 provides a comparison of influenza HA protein expression (as measured using an immunofluorescence assay) in cells transfected with either (i) a HA-encoding polynucleotide comprising the UTRs described herein (i.e., HA-5′-UTR and HA-3′-UTR) (right panel) (“mod-mRNA”) or (ii) a corresponding polynucleotide lacking the UTRs (middle panel) (“cont-mRNA”). Non-transfected cells were used as control (left panel).

FIG. 3 provides a comparison of influenza HA protein expression (as measured using Western blot) in cells transfected with either (i) a HA-encoding polynucleotide comprising the UTRs described herein (i.e., HA-5′-UTR and HA-3′-UTR) (last column) (“mod-mRNA”) or (ii) a corresponding polynucleotide lacking the UTRs (middle column) (“cont-mRNA”). Non-transfected cells were used as control (1^{st} column) (“mock”). HA protein expression was measured at both 6 hours and 24 hours post transfection.

FIG. 4 provides a comparison of GFP expression (as measured using immunofluorescence microscopy) in cells transfected with either (i) an GFP-encoding polynucleotide comprising the UTRs described herein (i.e., HA-5′-UTR and HA-3′-UTR) (middle column) (“mod-mRNA”) or (ii) a corresponding polynucleotide lacking the UTRs (last column) (“cont-mRNA”). Non-transfected cells were used as control (1^{st} column) (“mock”). GFP expression was measured at 6 hours (top row), 12 hours (middle row), and 24 hours (bottom row) post-transfection.

FIG. 5 provides a comparison of GFP expression (as measured using Western blot) in cells transfected with either (i) an GFP-encoding polynucleotide comprising the UTRs described herein (i.e., HA-5′-UTR and HA-3′-UTR) (last column) (“mod-mRNA”) or (ii) a corresponding polynucleotide lacking the UTRs (middle column) (“cont-mRNA”). Non-transfected cells were used as control (1^{st} column) (“mock”). The cells were transfected with two different concentrations of the polynucleotides: 1 μg or 3 μg.

FIG. 6 provides a comparison of the mean fluorescence intensity of GFP expression (as measured using flow cytometer) in cells transfected with either (i) an GFP-encoding polynucleotide comprising the UTRs described herein (i.e., HA-5′-UTR and HA-3′-UTR) (right panel) (“mod-mRNA”) or (ii) a corresponding polynucleotide lacking the UTRs (middle panel) (“cont-mRNA”). Non-transfected cells were used as control (left panel) (“mock”).

FIG. 7 provides a comparison of luciferase (luc) expression (as measured with IVIS imaging) in cells transfected with either (i) a luc-encoding polynucleotide comprising the UTRs described herein (i.e., HA-5′-UTR and HA-3′-UTR) (last column) (“mod-mRNA”) or (ii) a corresponding polynucleotide lacking the UTRs (middle column) (“cont-mRNA”). Non-transfected cells were used as control (1^{st} column) (“mock”). The cells were transfected with two different concentrations of the polynucleotides: 1 μg (top row) or 3 μg (bottom row).

FIG. 8 provides a comparison of luciferase (luc) expression (as measured with Western blot) in cells transfected with either (i) a luc-encoding polynucleotide comprising the UTRs described herein (i.e., HA-5′-UTR and HA-3′-UTR) (last column) (“mod-mRNA”) or (ii) a corresponding polynucleotide lacking the UTRs (middle column) (“cont-mRNA”). Non-transfected cells were used as control (1^{st} column) (“mock”). Luc expression was measured at 6 hours and 24 hours post-transfection.

FIG. 9 provides a comparison of luciferase (luc) expression in mice that received an administration of either (i) a luc-encoding polynucleotide comprising the UTRs described herein (i.e., HA-5′-UTR and HA-3′-UTR) (“3”) or (ii) a corresponding polynucleotide lacking the UTRs (“2”). Non-treated animals were used as control (“1”). Each row represents images of the mice taken from a different perspective. Luc expression was measured at 6 hours, 1 day, 2 days, 3 days, 4 days, 5 days, and 6 days after administration.

FIG. 10 provides a comparison of GFP expression (as measured using immunofluorescence microscopy) in cells transfected with either (i) a GFP-encoding polynucleotide comprising the UTRs described herein (i.e., HA-5′-UTR and HA-3′-UTR) (“mod-mRNA”) or (ii) a corresponding polynucleotide comprising control UTR sequences as described in Example 6 (“cont-UTR-mRNA”). The cells were transfected with the polynucleotides at three different concentrations: 0.5 μg, 1 μg, and 3 μg. Non-transfected cells were used as control (“mock”). GFP expression was measured at 6 hours (top row), 12 hours (middle row), and 24 hours (bottom row) post-transfection.

FIG. 11 provides a comparison of GFP expression (as measured using Western blot) in cells transfected with either (i) a GFP-encoding polynucleotide comprising the UTRs described herein (i.e., HA-5′-UTR and HA-3′-UTR) (“mod-mRNA”) or (ii) a corresponding polynucleotide comprising control UTR sequences as described in Example 6 (“cont-UTR-mRNA”). The cells were transfected with the polynucleotides at three different concentrations: 0.5 μg, 1 μg, and 3 μg. Non-transfected cells were used as control (“mock”). GFP expression was measured at 6 hours (top row), 12 hours (middle row), and 24 hours (bottom row) post-transfection.

FIG. 12 provides a comparison of coronavirus S protein expression (as measured using Western blot) in cells transfected with either (i) a S protein-encoding polynucleotide comprising the UTRs described herein (i.e., HA-5′-UTR and HA-3′-UTR) (“mod-mRNA”) or (ii) a corresponding polynucleotide lacking the UTRs (“cont-mRNA”). Non-transfected cells were used as control (“mock”). The S protein expression was measured at 6 hours and 24 hours post-transfection.

FIGS. 13 and 13B shows the body weight and survival, respectively, in animals that received one of the following treatments and subsequently challenged with an influenza infection: (i) no treatment (“mock”); (ii) nuclease free water alone (“NFW”); or (iii) polynucleotide encoding influenza HA protein and comprising the UTRs described herein (i.e., HA-5′-UTR and HA-3′-UTR) (“mHA”).

FIGS. 14A-14C show exemplary architectures of carrier units and micelles of the present disclosure. The exemplary carrier units comprise an optional tissue-specific targeting moiety, water soluble polymer, and cationic carrier unit (which can, respectively, interact with anionic payloads) (FIG. 14A). In some aspects, the cationic carrier and anionic payload are not tethered and interact electrostatically. FIG. 14B shows a schematic diagram of an anionic payload. In some aspects, the cationic carrier and anionic payload are tethered and interact electrostatically. FIG. 14C shows a schematic diagram of a micelle comprising a cationic carrier and anionic payload of FIGS. 14A and 14B.

FIGS. 15A-15E show exemplary compositions of cationic carrier units. FIG. 15A shows a carrier unit comprising a targeting moiety, water soluble polymer (e.g., polyethylene glycol), and a cationic carrier unit comprising 80 lysine residues, wherein 64 lysine residues are unmodifided (e.g., contain positively charged —NH3+) and wherein 16 lysine residues are modified for crosslinking (e.g., linked to a thiol, alkyl thiol, or lysine-thiol). FIG. 15B shows a carrier unit comprising a targeting moiety, water soluble polymer (e.g., polyethylene glycol), and a cationic carrier unit comprising 80 lysine residues, wherein 40 lysine residues are unmodifided (e.g., contain a positively charged amine group, e.g., —NH3+) and wherein 35 lysine residues are modified for crosslinking (e.g., linked to a thiol, alkyl thiol, or lysine-thiol) and wherein 5 lysine residues are modified to contain a hydrophobic moiety (e.g., a vitamin). FIG. 15C shows a carrier unit comprising a targeting moiety, water soluble polymer (e.g., polyethylene glycol), and a cationic carrier unit comprising 80 lysine residues, wherein 38 lysine residues are unmodifided (e.g., contain a positively charged amine group, e.g., —NH3+) and wherein 23 lysine residues are modified for crosslinking (e.g., linked to a thiol, alkyl thiol, or lysine-thiol) and wherein 19 lysine residues are modified to contain a hydrophobic moiety (e.g., a vitamin). FIG. 15D shows a carrier unit comprising a targeting moiety, water soluble polymer (e.g., polyethylene glycol), and a cationic carrier unit comprising 80 lysine residues, wherein 32 lysine residues are unmodifided (e.g., contain a positively charged amine group, e.g., —NH3+) and wherein 16 lysine residues are modified for crosslinking (e.g., linked to a thiol, alkyl thiol, or lysine-thiol) and wherein 32 lysine residues are modified to contain a hydrophobic moiety (e.g., a vitamin). FIG. 15E shows a carrier unit comprising a targeting moiety, water soluble polymer (e.g., polyethylene glycol), and a cationic carrier unit comprising 80 lysine residues, wherein 63 lysine residues are unmodifided (e.g., contain a positively charged amine group, e.g., —NH3+) and wherein 17 lysine residues are modified to contain a hydrophobic moiety (e.g., a vitamin).

FIGS. 16A and 16B provide comparison of GFP expression (as measured using fluorescent microscopy) in HEK293T cells transfected with either (i) a GFP-encoding polynucleotide comprising the UTRs described herein (i.e., HA-5′-UTR and HA-3′-UTR) (“HA-UTR-EGFP”) or (ii) a corresponding polynucleotide comprising human beta globin (hBg) UTR sequences as described in Example 9 (“hBg-UTR-EGFP”). Non-transfected cells were used as control (“Mock”). FIG. 16A shows protein expression at 6 hours (“6hpt”; top panel) and 24 hours (“24hpt”; bottom panel) after transfection. FIG. 16B shows protein expression at 48 hours (“48hpt”; top panel) and 72 hours (“72hpt”; bottom panel) after transfection. Scale bar=250 μm.

FIG. 17 provides a comparison of GFP expression (as measured using Western blot) in HEK293T cells transfected with either (i) a GFP-encoding polynucleotide comprising the UTRs described herein (i.e., HA-5′-UTR and HA-3′-UTR) (“HA-UTR-EGFP”) or (ii) a corresponding polynucleotide comprising human beta globin (hBg) UTR sequences as described in Example 9 (“hBg-UTR-EGFP”). Non-transfected cells were used as control (“Mock”). Protein expression was measured at 6 hours (“6hpt”), 24 hours (“24hpt”), 48 hours (“48hpt”), and 72 hours (“72hpt”) after transfection. β-actin expression is shown as an internal control.

FIG. 18 provides a comparison of the mean fluorescence intensity (MFI) of GFP expression (as measured using flow cytometry) in HEK293T cells transfected with either (i) a GFP-encoding polynucleotide comprising the UTRs described herein (i.e., HA-5′-UTR and HA-3′-UTR) (“HA-UTR-EGFP”) or (ii) a corresponding polynucleotide comprising human beta globin (hBg) UTR sequences as described in Example 9 (“hBg-UTR-EGFP”). Non-transfected cells were used as control (“Mock”). The GFP MFI values shown correspond to the positive gate value of GFP expression in each group. Statistical analysis was performed using the Student's t-test (two-tailed, unpaired; ***P<0.0001).

FIG. 19 provides bioluminescence imaging analysis of luciferase expression in HEK293T cells transfected with either (i) a luciferase-encoding polynucleotide comprising the UTRs described herein (i.e., HA-5′-UTR and HA-3′-UTR) (“HA-UTR-Fluc”) or (ii) a corresponding polynucleotide comprising human beta globin (hBg) UTR sequences as described in Example 9 (“hBg-UTR-Fluc”). Non-transfected cells were used as control (“Mock”). Protein expression was measured at 6 hours (“6hpt”), 24 hours (“24hpt”), 48 hours (“48hpt”), and 72 hours (“72hpt”) after transfection.

FIG. 20 provides comparison of luciferase expression (as measured using Western blot) in HEK293T cells transfected with either (i) a GFP-encoding polynucleotide comprising the UTRs described herein (i.e., HA-5′-UTR and HA-3′-UTR) (“HA-UTR-EGFP”) or (ii) a corresponding polynucleotide comprising human beta globin (hBg) UTR sequences as described in Example 9 (“hBg-UTR-EGFP”). Non-transfected cells were used as control (“Mock”). Protein expression was measured at 6 hours (“6hpt”), 24 hours (“24hpt”), 48 hours (“48hpt”), and 72 hours (“72hpt”) after transfection. 3-actin expression is shown as an internal control.

FIGS. 21A and 21B provide additional comparison of body weight and survival, respectively, in animals that received one of the following treatments and subsequently challenged with an influenza infection: (i) no treatment (“mock”; asterisk); (ii) nuclease free water alone (“NFW”; triangle); or (iii) polynucleotide encoding influenza HA protein and comprising the UTRs described herein (i.e., HA-5′-UTR and HA-3′-UTR) (“IVJ+mHA”; square”) (2.5 μg/mouse). The HA-UTR-HA was formulated in a mRNA delivery reagent as described in Example 8.

FIG. 22 provides Western blot analysis of influenza hemagglutinin (HA) protein expression in HEK293T cells transfected with a polynucleotide encoding the HA protein of A/H3N2 strain either (i) with the UTRs described herein (i.e., HA-5′-UTR and HA-3′-UTR) (“HA-UTR-H3”) or (ii) without (“H3-ORF”). Non-transfected cells were used as control (“mock”). Protein expression was measured at 6 hours (“6hpt”), 24 hours (“24hpt”), 48 hours (“48hpt”), and 72 hours (“72hpt”) after transfection. β-actin expression is shown as an internal control.

FIG. 23 provides Western blot analysis of influenza hemagglutinin (HA) protein expression in HEK293T cells transfected with a polynucleotide encoding the HA protein of B/Yamagata strain either (i) with the UTRs described herein (i.e., HA-5′-UTR and HA-3′-UTR) (“HA-UTR”) or (ii) without (“ORF”). Non-transfected cells were used as control (“mock”). Protein expression was measured at 6 hours (“6hpt”), 24 hours (“24hpt”), 48 hours (“48hpt”), and 72 hours (“72hpt”) after transfection. β-actin expression is shown as an internal control.

FIG. 24 provides Western blot analysis showing iduronate-2-sulfatase (IDS) expression (both the precursor and mature forms) in HEK293T cells transfected with a polynucleotide encoding the IDS protein either (i) with the UTRs described herein (i.e., HA-5′-UTR and HA-3′-UTR) (“HA-UTR-IDS”) or (ii) without (“IDS-ORF”). The cells were transfected with the polynucleotides at two different doses: 1 μg (top panel) or 3 μg (bottom panel). Non-transfected cells were used as control (“Mock”). Protein expression was measured at 6 hours (“6hpt”), 24 hours (“24hpt”), 48 hours (“48hpt”), and 72 hours (“72hpt”) after transfection. β-actin expression is shown as an internal control.

FIG. 25 provides Western blot analysis showing NYESO1 protein expression in HEK293 T cells transfected with a polynucleotide encoding the NYESO1 protein either (i) with the UTRs described herein (i.e., HA-5′-UTR and HA-3′-UTR) (“HA-UTR-NYESO1”) or (ii) without (“NYESO1”). Non-transfected cells were used as control (“Mock”). GADPH expression is shown as an internal control.

FIG. 26 provides Western blot analysis showing MAGEA3 protein expression in HEK293 T cells transfected with a polynucleotide encoding the MAGEA3 protein either (i) with the UTRs described herein (i.e., HA-5′-UTR and HA-3′-UTR) (“HA-UTR-MAGEA3”) or (ii) without (“MAGEA3”). Non-transfected cells were used as control (“Mock”). GADPH expression is shown as an internal control.

### DETAILED DESCRIPTION OF THE DISCLOSURE

The present disclosure is directed to a polynucleotide (e.g., isolated polynucleotide) which has been modified to exhibit one or more improved properties. Accordingly, the polynucleotides of the present disclosure differ (e.g., structurally and/or functionally) from a reference polynucleotide that exists in nature. For instance, in some aspects, a polynucleotide of the present disclosure comprises (i) an open reading frame (ORF) encoding a protein of interest, and (ii) one or more additional components (e.g., UTR sequences provided herein), which are capable of increasing the expression of the encoded protein when translated.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

As used herein, the terms “ug” and “uM” are used interchangeably with “μg” and “μM,” respectively.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, ‘a’ represents adenine, ‘c’ represents cytosine, ‘g’ represents guanine, ‘t’ represents thymine, and ‘u’ represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV” includes a derivative of a known AAV. In some aspects, an “AAV” includes a modified or an artificial AAV.

The terms “administration,” “administering,” and grammatical variants thereof refer to introducing a composition, such as a polynucleotide of the present disclosure, into a subject via a pharmaceutically acceptable route. The introduction of a composition, such as a micelle comprising a polynucleotide of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least 70% identical, at least 80% identical, at least 90% identical, or at least 95% identical to one another. In some aspects, two or more sequences are said to be “highly conserved” if they are about 70% identical, about 80% identical, about 90% identical, about 95%, about 98%, or about 99% identical to one another. In some aspects, two or more sequences are said to be “conserved” if they are at least 30% identical, at least 40% identical, at least 50% identical, at least 60% identical, at least 70% identical, at least 80% identical, at least 90% identical, or at least 95% identical to one another. In some aspects, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence can apply to the entire length of a polynucleotide or polypeptide or can apply to a portion, region or feature thereof.

The term “derived from,” as used herein, refers to a component that is isolated from or made using a specified molecule or organism, or information (e.g., amino acid or nucleic acid sequence) from the specified molecule or organism. For example, a nucleic acid sequence that is derived from a second nucleic acid sequence can include a nucleotide sequence that is identical or substantially similar to the nucleotide sequence of the second nucleic acid sequence. In the case of nucleotides or polypeptides, the derived species can be obtained by, for example, naturally occurring mutagenesis, artificial directed mutagenesis or artificial random mutagenesis. The mutagenesis used to derive nucleotides or polypeptides can be intentionally directed or intentionally random, or a mixture of each. The mutagenesis of a nucleotide or polypeptide to create a different nucleotide or polypeptide derived from the first can be a random event (e.g., caused by polymerase infidelity) and the identification of the derived nucleotide or polypeptide can be made by appropriate screening methods, e.g., as discussed herein. In some aspects, a nucleotide or amino acid sequence that is derived from a second nucleotide or amino acid sequence has a sequence identity of at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% to the second nucleotide or amino acid sequence, respectively, wherein the first nucleotide or amino acid sequence retains the biological activity of the second nucleotide or amino acid sequence.

As used herein, a “coding region” or “coding sequence” is a portion of polynucleotide which consists of codons translatable into amino acids (e.g., open reading frame). Although a “stop codon” (TAG, TGA, or TAA) is typically not translated into an amino acid, it can be considered to be part of a coding region, but any flanking sequences, for example, UTRs, promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The terms “complementary” and “complementarity” refer to two or more oligomers (i.e., each comprising a nucleobase sequence), or between an oligomer and a target gene, that are related with one another by Watson-Crick base-pairing rules. For example, the nucleobase sequence “T-G-A (5′43′),” is complementary to the nucleobase sequence “A-C-T (3′4 5′).” Complementarity can be “partial,” in which less than all of the nucleobases of a given nucleobase sequence are matched to the other nucleobase sequence according to base pairing rules. For example, in some aspects, complementarity between a given nucleobase sequence and the other nucleobase sequence can be about 70%, about 75%, about 80%, about 85%, about 90%, or about 95%. Accordingly, in certain aspects, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a target nucleic acid sequence. Or, there can be “complete” or “perfect” (100%) complementarity between a given nucleobase sequence and the other nucleobase sequence to continue the example. In some aspects, the degree of complementarity between nucleobase sequences has significant effects on the efficiency and strength of hybridization between the sequences.

The term “downstream” refers to a nucleotide sequence that is located 3′ to a reference nucleotide sequence. In certain aspects, downstream nucleotide sequences relate to sequences that follow the starting point of transcription. For example, the translation initiation codon of a gene is located downstream of the start site of transcription.

The terms “excipient” and “carrier” are used interchangeably and refer to an inert substance added to a pharmaceutical composition to further facilitate administration of a compound, e.g., a miRNA inhibitor of the present disclosure.

The term “expression,” as used herein, refers to a process by which a polynucleotide produces a gene product, e.g., RNA or a polypeptide (e.g., therapeutic protein, e.g., coronavirus protein). It includes without limitation transcription of the polynucleotide into micro RNA binding site, small hairpin RNA (shRNA), small interfering RNA (siRNA), or any other RNA product. It includes, without limitation, transcription of the polynucleotide into messenger RNA (mRNA), and the translation of mRNA into a polypeptide. Expression produces a “gene product.” As used herein, a gene product can be, e.g., a nucleic acid, such as an RNA produced by transcription of a gene. As used herein, a gene product can be either a nucleic acid, RNA or miRNA produced by the transcription of a gene, or a polypeptide which is translated from a transcript. Gene products described herein further include nucleic acids with post transcriptional modifications, e.g., polyadenylation or splicing, or polypeptides with post translational modifications, e.g., phosphorylation, methylation, glycosylation, the addition of lipids, association with other protein subunits, or proteolytic cleavage.

In some aspects, polymeric molecules are considered to be “homologous” to one another if at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of the monomers in the molecule are identical (exactly the same monomer) or are similar (conservative substitutions). The term “homologous” necessarily refers to a comparison between at least two sequences (e.g., polynucleotide sequences).

In the context of the present disclosure, substitutions (even when they are referred to as amino acid substitution) are conducted at the nucleic acid level, i.e., substituting an amino acid residue with an alternative amino acid residue is conducted by substituting the codon encoding the first amino acid with a codon encoding the second amino acid.

“Heterologous” in reference to a polypeptide moiety or polynucleotide moiety that is part of a larger polypeptide or polynucleotide, respectively, describes a polypeptide or polynucleotide that originates from a different polypeptide or polynucleotide than the remaining part of the polypeptide or polynucleotide molecule. The additional heterologous component of the polypeptide or polynucleotide can originate from the same organism as the remaining polypeptide or polynucleotide, respectively, described herein, or the additional components can be from a different organism. For instance, a heterologous polypeptide can be synthetic, or derived from a different species, different cell type of an individual, or the same or different type of cell of distinct individuals. As described herein, a protein (or polypeptide) encoded by an ORF of a polynucleotide described herein is heterologous to the UTRs (e.g., HA-5′-UTR and HA-3′-UTR) of the polynucleotide.

As used herein, the term “identity” (e.g., sequence identity) refers to the overall monomer conservation between polymeric molecules, e.g., between polynucleotide molecules. The term “identical” without any additional qualifiers, e.g., polynucleotide A is identical to polynucleotide B, implies the polynucleotide sequences are 100% identical (100% sequence identity). Describing two sequences as, e.g., “70% identical,” is equivalent to describing them as having, e.g., “70% sequence identity.”

Calculation of the percent identity of two polypeptide or polynucleotide sequences, for example, can be performed by aligning the two sequences for optimal comparison purposes (e.g., gaps can be introduced in one or both of a first and a second polypeptide or polynucleotide sequences for optimal alignment and non-identical sequences can be disregarded for comparison purposes). In certain aspects, the length of a sequence aligned for comparison purposes is at least 30%, at least 40%, at least 50%, at least 60%, at least 70%, at least 80%, at least 90%, at least 95%, or 100% of the length of the reference sequence. The amino acids at corresponding amino acid positions, or bases in the case of polynucleotides, are then compared.

When a position in the first sequence is occupied by the same amino acid or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm.

Suitable software programs that can be used to align different sequences (e.g., polynucleotide sequences) are available from various sources. One suitable program to determine percent sequence identity is b12seq, part of the BLAST suite of program available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Sequence alignments can be conducted using methods known in the art such as MAFFT, Clustal (ClustalW, Clustal X or Clustal Omega), MUSCLE, etc.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In certain aspects, the percentage identity (% ID) or of a first amino acid sequence (or nucleic acid sequence) to a second amino acid sequence (or nucleic acid sequence) is calculated as % ID=100×(Y/Z), where Y is the number of amino acid residues (or nucleobases) scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence.

One skilled in the art will appreciate that the generation of a sequence alignment for the calculation of a percent sequence identity is not limited to binary sequence-sequence comparisons exclusively driven by primary sequence data. It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity can be curated either automatically or manually.

As used herein, the terms “isolated,” “purified,” “extracted,” and grammatical variants thereof are used interchangeably and refer to the state of a preparation of desired composition of the present disclosure, e.g., a polynucleotide of the present disclosure, that has undergone one or more processes of purification. In some aspects, isolating or purifying as used herein is the process of removing, partially removing (e.g., a fraction) of a composition of the present disclosure, e.g., a polynucleotide of the present disclosure (e.g., comprising an ORF, a HA-5′-UTR, and a HA-3′-UTR) from a sample containing contaminants.

In some aspects, an isolated composition has no detectable undesired activity or, alternatively, the level or amount of the undesired activity is at or below an acceptable level or amount. In other aspects, an isolated composition has an amount and/or concentration of desired composition of the present disclosure, at or above an acceptable amount and/or concentration and/or activity. In other aspects, the isolated composition is enriched as compared to the starting material from which the composition is obtained. This enrichment can be by at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 99.9%, at least about 99.99%, at least about 99.999%, at least about 99.9999%, or greater than 99.9999% as compared to the starting material.

In some aspects, isolated preparations are substantially free of residual biological products. In some aspects, the isolated preparations are 100% free, at least about 99% free, at least about 98% free, at least about 97% free, at least about 96% free, at least about 95% free, at least about 94% free, at least about 93% free, at least about 92% free, at least about 91% free, or at least about 90% free of any contaminating biological matter. Residual biological products can include abiotic materials (including chemicals) or unwanted nucleic acids, proteins, lipids, or metabolites.

The term “linked” as used herein refers to a first amino acid sequence or polynucleotide sequence covalently or non-covalently joined to a second amino acid sequence or polynucleotide sequence, respectively. The first amino acid or polynucleotide sequence can be directly joined or juxtaposed to the second amino acid or polynucleotide sequence or alternatively an intervening sequence can covalently join the first sequence to the second sequence. The term “linked” means not only a fusion of a first polynucleotide sequence to a second polynucleotide sequence at the 5′-end or the 3′-end, but also includes insertion of the whole first polynucleotide sequence (or the second polynucleotide sequence) into any two nucleotides in the second polynucleotide sequence (or the first polynucleotide sequence, respectively). The first polynucleotide sequence can be linked to a second polynucleotide sequence by a phosphodiester bond or a linker. The linker can be, e.g., a polynucleotide.

As used herein, the terms “modulate,” “modify,” and grammatical variants thereof, generally refer when applied to a specific concentration, level, expression, function or behavior, to the ability to alter, by increasing or decreasing, e.g., directly or indirectly promoting/stimulating/up-regulating or interfering with/inhibiting/down-regulating the specific concentration, level, expression, function or behavior, such as, e.g., to act as an antagonist or agonist. In some instances, a modulator can increase and/or decrease a certain concentration, level, activity or function relative to a control, or relative to the average level of activity that would generally be expected or relative to a control level of activity.

“Nucleic acid,” “nucleic acid molecule,” “nucleotide sequence,” “polynucleotide,” and grammatical variants thereof are used interchangeably and refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Single stranded nucleic acid sequences refer to single-stranded DNA (ssDNA) or single-stranded RNA (ssRNA). Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, supercoiled DNA and chromosomes. In discussing the structure of particular double-stranded DNA molecules, sequences can be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation. DNA includes, but is not limited to, cDNA, genomic DNA, plasmid DNA, synthetic DNA, and semi-synthetic DNA. A “nucleic acid composition” of the disclosure comprises one or more nucleic acids as described herein. As described herein, a polynucleotide of the present disclosure comprises DNA, RNA, or both. In some aspects, the term “polynucleotide” includes polydeoxyribonucleotides (containing 2-deoxy-D-ribose), polyribonucleotides (containing D-ribose), including tRNA, rRNA, shRNA, siRNA, miRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA.

The terms “pharmaceutically acceptable carrier,” “pharmaceutically acceptable excipient,” and grammatical variations thereof, encompass any of the agents approved by a regulatory agency of the U.S. Federal government or listed in the U.S. Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “pharmaceutical composition” refers to one or more of the compounds described herein, such as, e.g., a polynucleotide of the present disclosure, mixed or intermingled with, or suspended in one or more other chemical components, such as pharmaceutically acceptable carriers and excipients.

The terms “polypeptide,” “peptide,” and “protein” are used interchangeably herein to refer to polymers of amino acids of any length, e.g., that are encoded by a polynucleotide described herein. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art. The term “polypeptide,” as used herein, refers to proteins, polypeptides, and peptides of any size, structure, or function.

Polypeptides include gene products, naturally occurring polypeptides, synthetic polypeptides, homologs, orthologs, paralogs, fragments and other equivalents, variants, and analogs of the foregoing.

A polypeptide can be a single polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid. In some aspects, a “peptide” can be less than or equal to about 50 amino acids long, e.g., about 5, about 10, about 15, about 20, about 25, about 30, about 35, about 40, about 45, or about 50 amino acids long.

The terms “prevent,” “preventing,” and variants thereof as used herein, refer partially or completely delaying onset of an disease, disorder and/or condition; partially or completely delaying onset of one or more symptoms, features, or clinical manifestations of a particular disease, disorder, and/or condition; partially or completely delaying onset of one or more symptoms, features, or manifestations of a particular disease, disorder, and/or condition; partially or completely delaying progression from a particular disease, disorder and/or condition; and/or decreasing the risk of developing pathology associated with the disease, disorder, and/or condition. In some aspects, preventing an outcome is achieved through prophylactic treatment.

As used herein, the terms “promoter” and “promoter sequence” are interchangeable and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. In some aspects, a promoter that can be used with the present disclosure includes a tissue specific promoter.

As used herein, “prophylactic” refers to a therapeutic or course of action used to prevent the onset of a disease or condition, or to prevent or delay a symptom associated with a disease or condition.

As used herein, a “prophylaxis” refers to a measure taken to maintain health and prevent the onset of a disease or condition, or to prevent or delay a symptom associated with a disease or condition.

As used herein, the term “gene regulatory region” or “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, or stem-loop structures. If a coding region is intended for expression in a eukaryotic cell, a polyadenylation signal and transcription termination sequence will usually be located 3′ to the coding sequence.

In some aspects, a polynucleotide described herein can include a promoter and/or other expression (e.g., transcription) control elements (e.g., HA-5′-UTR and HA-3′-UTR) operably associated with one or more coding regions. In an operable association a coding region for a gene product is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other expression control elements, besides a promoter, for example enhancers, operators, repressors, and transcription termination signals, can also be operably associated with a coding region to direct gene product expression.

As used herein, the term “similarity” refers to the overall relatedness between polymeric molecules, e.g. between polynucleotide molecules. Calculation of percent similarity of polymeric molecules to one another can be performed in the same manner as a calculation of percent identity, except that calculation of percent similarity takes into account conservative substitutions as is understood in the art. It is understood that percentage of similarity is contingent on the comparison scale used, i.e., whether the nucleic acids are compared, e.g., according to their evolutionary proximity, charge, volume, flexibility, polarity, hydrophobicity, aromaticity, isoelectric point, antigenicity, or combinations thereof.

The terms “subject,” “patient,” “individual,” and “host,” and variants thereof are used interchangeably herein and refer to any mammalian subject, including without limitation, humans, domestic animals (e.g., dogs, cats and the like), farm animals (e.g., cows, sheep, pigs, horses and the like), and laboratory animals (e.g., monkey, rats, mice, rabbits, guinea pigs and the like) for whom diagnosis, treatment, or therapy is desired, particularly humans. The methods described herein are applicable to both human therapy and veterinary applications.

As used herein, the phrase “subject in need thereof” includes subjects, such as mammalian subjects, that would benefit from administration of a polynucleotide of the disclosure.

As used herein, the term “therapeutically effective amount” or “effective amount” is the amount of reagent or pharmaceutical compound comprising a polynucleotide of the present disclosure (e.g., comprising an ORF, HA-5′-UTR, and HA-3′-UTR) that is sufficient to a produce a desired therapeutic effect, pharmacologic and/or physiologic effect on a subject in need thereof. A therapeutically effective amount can be a “prophylactically effective amount” as prophylaxis can be considered therapy.

The terms “treat,” “treatment,” or “treating,” as used herein refers to, e.g., the reduction in severity of a disease or condition; the reduction in the duration of a disease course; the amelioration or elimination of one or more symptoms associated with a disease or condition (e.g., diabetes); the provision of beneficial effects to a subject with a disease or condition, without necessarily curing the disease or condition. The term also includes prophylaxis or prevention of a disease or condition or its symptoms thereof.

As used herein, the term “untranslated region” or “UTR” refers to a region of a gene that is transcribed but not translated. The “5′-UTR” starts at the transcription start site and continues to the start codon but does not include the start codon; whereas, the “3′-UTR” starts immediately following the stop codon and continues until the transcriptional termination signal. There is growing body of evidence about the regulatory roles played by the UTRs in terms of stability of the nucleic acid molecule and translation. The regulatory features of a UTR can be incorporated into a polynucleotide described herein to enhance the stability of the molecule. The specific features can also be incorporated to ensure controlled down-regulation of the transcript in case they are misdirected to undesired organs sites.

The term “upstream” refers to a nucleotide sequence that is located 5′ to a reference nucleotide sequence.

### II. Modified Polynucleotides

### II.A. Untranslated Regions (UTRs)

A polynucleotide (e.g., isolated polynucleotide) of the present disclosure has been modified to comprise (i) an open reading frame (ORF) encoding a protein of interest, and (ii) one or more additional components, wherein the additional components are capable of improving one or more properties of the polynucleotide. As described herein, in some aspects, the one or more additional components comprise: (i) a 5′-untranslated region element (5′-UTR) of an influenza hemagglutinin (HA) protein (or a functional fragment thereof) (also referred to herein as “HA-5′-UTR”), (ii) a 3′-untranslated region element (3′-UTR) of an influenza hemagglutinin (HA) protein (or a functional fragment thereof) (also referred to herein as “HA-3′-UTR”), or (iii) both (i) and (ii).

Accordingly, in some aspects, a polynucleotide (e.g., isolated polynucleotide) of the present disclosure comprises (i) an ORF encoding a protein of interest and (ii) a HA-5′-UTR, wherein the protein of interest is heterologous to the HA-5′-UTR (i.e., not the same influenza HA protein). In some aspects, a polynucleotide (e.g., isolated polynucleotide) comprises (i) an ORF encoding a protein of interest and (ii) a HA-3′-UTR, wherein the protein of interest is heterologous to the HA-3′-UTR (i.e., not the same influenza HA protein). In some aspects, a polynucleotide described herein comprises (i) an ORF encoding a protein of interest, (ii) a HA-5′-UTR, and (iii) a HA-3′-UTR, wherein the protein of interest is heterologous to the HA-5′-UTR and/or the HA-3′-UTR.

In some aspects, the HA-5′-UTR flanks (i.e., adjacent to) the 5′-end of the ORF. In some aspects, one or more additional components (e.g., further discussed in Section II.C) are present in between the HA-5′-UTR and the 5′-end of the ORF. In some aspects, the HA-3′-UTR flanks the 3′-end of the ORF. In some aspects, one or more additional components (e.g., further discussed in Section II.C) are present in between the 3′-end of the ORF and the HA-3′-UTR.

In some aspects, the HA-5′-UTR comprises the nucleic acid sequence set forth in SEQ ID NO: 13 (AGCAAAAGCAGGGGAAAATAAAAGCAACAAAA). In certain aspects, the HA-5′-UTR described herein consists of the nucleic acid sequence set forth in SEQ ID NO: 13 (AGCAAAAGCAGGGGAAAATAAAAGCAACAAAA). In some aspects, the HA-3′-UTR comprises the nucleic acid sequence set forth in SEQ ID NO: 14 (CATTAGGATTTCAGAAGCATGAGAAAAACACCCTTGTTTCTACT). In some aspects, the HA-3′-UTR consists of the nucleic acid sequence set forth in SEQ ID NO: 14 (CATTAGGATTTCAGAAGCATGAGAAAAACACCCTTGTTTCTACT).

As demonstrated herein, Applicant has identified that the inclusion of one or more of the UTR sequences provided (e.g., HA-5′-UTR and HA-3′-UTR) herein in constructing a polynucleotide (e.g., such as those described herein) can increase the expression of the encoded protein when translated. In some aspects, the expression of the protein is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least 25-fold, at least about 30-fold, at least about 35-fold, at least about 40-fold, at least about 45-fold, at least about 50-fold, at least about 75-fold, or at least about 100-fold or more, compared to a reference expression (e.g., expression of the protein when encoded by a polynucleotide lacking the HA-5′-UTR and/or HA-3′-UTR).

As further demonstrated herein, in some aspects, the UTR sequences provided herein (e.g., SEQ ID NO: 13 or SEQ ID NO: 14) are capable of increasing the expression of a protein to a greater extent compared to other UTR sequences, including those known in the art. For instance, in some aspects, compared to other UTR sequences (e.g., 2hBgUTR (SEQ ID NO: 15) disclosed in U.S. Pat. No. 10,301,368 B2, which is incorporated herein by reference in its entirety), the UTR sequences described herein (e.g., SEQ ID NO: 13 or SEQ ID NO: 14) increase the expression of an encoded protein by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least 25-fold, at least about 30-fold, at least about 35-fold, at least about 40-fold, at least about 45-fold, at least about 50-fold, at least about 75-fold, or at least about 100-fold or more.

UTRs can have features that provide a regulatory role, e.g., increased or decreased stability, localization and/or translation efficiency. Not to be bound by any one theory, in some aspects, the UTR sequences of the present disclosure (e.g., SEQ ID NO: 13 or SEQ ID NO: 14) can increase the expression of a protein of interest by improving the stability, localization, and/or translation efficiency of the polynucleotide encoding the protein of interest. In certain aspects, the UTR sequences described herein have greater ability to regulate such aspects (e.g., stability, localization, and/or translation efficiency) of a polynucleotide compared to other UTR sequences, including those known in the art (e.g., 2hBgUTR; SEQ ID NO: 15).

As is apparent from the present disclosure, in some aspects, a polynucleotide described herein comprises a single UTR sequence, e.g., comprising or consisting of the nucleotide sequence set forth in SEQ ID NO: 13 or SEQ ID NO: 14. In some aspects, a polynucleotide described herein comprises multiple (e.g., 2 or more) UTRs, wherein at least one of the UTRs is selected from a HA-5′-UTR and/or HA-3′-UTR of the present disclosure (e.g., SEQ ID NO: 13 or SEQ ID NO: 14). In certain aspects, a polynucleotide described herein comprises multiple (e.g., 2 or more) 5′-UTRs. In some aspects, a polynucleotide described herein comprises multiple (e.g., 2 or more) 3′-UTRs. In further aspects, a polynucleotide described herein comprises multiple (e.g., 2 or more) 5′-UTRs and multiple (e.g., 2 or more) 3′-UTRs. For polynucleotides comprising multiple UTRs, in some aspects, each of the UTRs have the same nucleotide sequence. Not to be bound by any one theory, in some aspects, the inclusion of repetitive UTR sequences can help further increase the stability and/or translation efficiency of the associated polynucleotide. In certain aspects, one or more of the multiple UTRs have different nucleotide sequences. In further aspects, each of multiple UTRs have different nucleotide sequences.

Unless indicated otherwise, the UTRs of the present disclosure (e.g., SEQ ID NO: 13 or SEQ ID NO: 14) can be used in combination with any suitable UTRs known in the art. In some aspects, the additional UTRs that can be used in combination with the UTRs of the present disclosure include those that are present in genes that are abundantly expressed in specific cells, tissues, and/or organs. By including such additional UTRs, in some aspects, a polynucleotide of the present disclosure can be preferentially expressed in specific cells, tissues, and/or organs, e.g., when administered to a subject. For example, additionally introducing a UTR (e.g., 5-UTR) of a mRNA expressed in the liver (e.g., albumin, serum amyloid A, Apolipoprotein A/B/E, transferrin, alpha fetoprotein, erythropoietin, or Factor VIII) can enhance the expression of a polynucleotide described herein in hepatic and/or liver cell lines. Non-limiting examples of such tissue-specific UTRs include those from: (a) muscle: myoD, myosin, myoglobin, myogenin, and herculin; (b) endothelial cells: Tie-1 and CD36; (c) myeloid cells: C/EBP, AML1, G-CSF, GM-CSF, CD11b, MSR, Fr-1, and i-NOS; (d) leukocytes: CD45 and CD18; (e) adipose tissue: CD36, GLUT4, ACRP30, and adiponectin; and (f) lung epithelial cells: SP-A/B/C/D.

Additional examples of UTRs that can be used in combination with those of the present disclosure (e.g., SEQ ID NO: 13 or SEQ ID NO: 14) include one or more 5′-UTR and/or 3′-UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human a or R actin); a GAPDH; a tubulin; a histone; a citric acid cycle enzyme; a topoisomerase (e.g., a 5′-UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H⁺-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); a nucleobindin (e.g., Nucb1); and combinations thereof.

### II.B. Open Reading Frame (ORF)

As described herein, in some aspects, a polynucleotide of the present disclosure comprises an ORF encoding a protein and one or more UTRs (e.g., SEQ ID NO: 13 or SEQ ID NO: 14) that can enhance the expression of the encoded protein. As further described herein, the encoded protein is heterologous to the one or more UTRs described herein. Accordingly, the encoded protein and the UTRs described herein (e.g., HA-5′-UTR and HA-3′-UTR) are not naturally found together (e.g., are not from the same strain of an influenza virus). Otherwise, any suitable proteins known in the art can be encoded using the polynucleotides of the present disclosure. For instance, in some aspects, the ORF of a polynucleotide described herein encodes a therapeutic protein. As used herein, the term “therapeutic protein” refers to any protein or polypeptide capable of exerting a therapeutic effect (e.g., inducing an immune response against the protein or polypeptide). As used herein, the term “therapeutic effect” refers to an effect that results in some benefit to a subject (e.g., a reduction, elimination, or prevention of a disease or abnormal condition, symptoms thereof, and/or side effects thereof).

In some aspects, the ORF of a polynucleotide described herein encodes a viral protein (i.e., therapeutic protein). In some aspects, the viral protein is derived from a coronavirus (“coronavirus protein”). In certain aspects, the coronavirus comprises SARS-CoV-1, SARS-CoV-2 (COVID-19), or both. In certain aspects, the coronavirus comprises Middle East respiratory syndrome-related coronavirus (MERS-CoV; also known as EMC/2012). Accordingly, in certain aspects, a polynucleotide (e.g., isolated polynucleotide) of the present disclosure comprises: (i) an ORF encoding a coronavirus protein, (ii) a 5′-untranslated region element (5′-UTR) of an influenza hemagglutinin (HA) protein (e.g., SEQ ID NO: 13), and (iii) a 3′-untranslated region element (3′-UTR) of an influenza hemagglutinin (HA) protein (e.g., SEQ ID NO: 14). As demonstrated herein, in some aspects, the 5′-UTR and/or the 3′-UTR increases the expression of the encoded coronavirus protein, when the polynucleotide is translated.

All members of the coronavirus family are enveloped viruses that possess long positive-sense, single-stranded RNA genomes ranging in size from 27 to 33 kb. The coronavirus genomes encode five major open reading frames (ORFs), including a 5′ frameshifted polyprotein (ORF1a/ORF1ab) and four canonical 3′ structural proteins, namely the spike (S), envelope (E), membrane (M), and nucleocapsid (N) proteins, which are common to all coronaviruses. The viral envelope consists of a lipid bilayer, in which the membrane (M), envelope (E) and spike (S) structural proteins are anchored. Chen et. al., J Med Virol 92(4): 418-423 (April 2020), which is herein incorporated by reference in its entirety. There are seven strains of human coronaviruses currently known: (i) human coronavirus 229E (HCoV-229E); (ii) human coronavirus OC43 (HCoV-OC43); (iii) severe acute respiratory syndrome coronavirus 1 (SARS-CoV-1); (iv) human coronavirus NL63 (HCoV-NL63, New Haven coronavirus); (v) human coronavirus HKU1; (vi) Middle East respiratory syndrome-related coronavirus (MERS-CoV, also known as novel coronavirus 2012 and HCoV-EMC); and (vii) severe acute respiratory syndrome coronavirus 2 (SARS-CoV-2), also known as 2019-nCoV, novel coronavirus 2019, or COVID19. Unless specified otherwise, the term “coronavirus,” as used herein, comprises all coronaviruses within the family Coronaviridae, such as the human coronaviruses described herein, including all mutants and variants thereof. In some aspects, the coronavirus is an alphacoronavirus, a betacoronavirus, a gammacoronavirus, a deltacoronavirus, or combinations thereof. Exemplary description of such coronaviruses are provided in, e.g., Krichel et al., Sci Adv 7(10):eabf1004 (March 2021), which is incorporated herein by reference in its entirety. In certain aspects, the coronavirus protein (e.g., spike protein) is derived from a bat coronavirus (BtCoV), such as that described in Tsuda et al., Arch Virol 157(12): 2349-55 (December 2012), which is incorporated herein by reference in its entirety. In some aspects, the coronavirus comprises zoonotic coronaviruses (i.e., jumped from an animal to human). See, e.g., Cohen et al., Science 371(6530): 735-741 (February 2021), which is incorporated herein by reference in its entirety.

In some aspects, the coronavirus protein comprises a spike protein (or a fragment thereof). For instance, in certain aspects, the ORF of a polynucleotide described herein (e.g., comprising one or more UTRs of the present disclosure) encodes the S1 spike protein (or a fragment thereof). In some aspects, the ORF encodes the receptor binding domain (RBD) of the S1 spike protein. In certain aspects, the ORF of a polynucleotide described herein encodes the S2 spike protein (or a fragment thereof). In certain aspects, the ORF of a polynucleotide described herein encodes the S2′ spike protein (or a fragment thereof) of a coronavirus. In some aspects, the ORF of a polynucleotide of the present disclosure encodes an envelope (E) protein (or a fragment thereof) of a coronavirus. In some aspects, the ORF of a polynucleotide described herein encodes a membrane (M) protein (or a fragment thereof) of a coronavirus.

In some aspects, the viral protein is derived from an influenza virus (“influenza protein”), wherein the influenza virus is heterologous to the one or more UTRs described herein. For instance, as described herein (see, e.g., Example 1), the HA-5′-UTR and HA-3′-UTR set forth in SEQ ID NOs: 13 and 14, respectively, were derived from the HA protein of the 2009 pandemic influenza virus (H1N1pdm09_A/Korea/01/09). Accordingly, when a polynucleotide described herein comprises the HA-5′-UTR and HA-3′-UTR set forth in SEQ ID NOs: 13 and 14, the ORF of the polynucleotide does not encode the HA protein from the H1N1pdm09_A/Korea/01/09. Instead, in some aspects, when a polynucleotide described herein comprises the HA-5′-UTR and HA-3′-UTR set forth in SEQ ID NOs: 13 and 14, the ORF encodes a non-HA influenza protein (e.g., such as those described herein) from H1N1pdm09_A/Korea/01/09. In certain aspects, when a polynucleotide described herein comprises the HA-5′-UTR and HA-3′-UTR set forth in SEQ ID NOs: 13 and 14, the ORF encodes a protein from a different influenza strain. Non-limiting examples of such influenza strains are known in the art. See, e.g., US Publ. No. 2007/0141078 A1; and Krammer et al., Nat Rev Dis Primers 4(1):3 (June 2018), each of which is incorporated herein by reference in its entirety.

The influenza virus is an enveloped virus comprising a negative-sense, single-stranded RNA genome that is segmented. There are four species of the influenza virus: (1) influenza A virus (IAV); (2) influenza B virus (IBV); (3) influenza C virus (ICV); and (4) influenza D virus (IDV). IAV and IBV have eight genome segments that encode 10 major proteins. ICV and IDV have seven genome segments that encode nine major proteins. Three segments encode three subunits of an RNA-dependent RNA polymerase (RdRp) complex: PB1, a transcriptase; PB2, which recognizes 5′ caps; and PA (P3 for ICV and IDV), an endonuclease. The matrix protein (M1) and membrane protein (M2) share a segment, as do the non-strucutral protein (NS1) and the nuclear export protein (NEP). For IAV and IBV, hemagglutinin (HA) and neuraminidase (NA) are encoded on one segment each, whereas ICV and IDV encode a hemagglutinin-esterase fusion (HEF) protein on one segment that merges the functions of HA and NA. The final genome segment encodes the viral nucleoprotein (NP). Influenza viruses also encode various accessory proteins, such as PB1-F2 and PA-X, that are expressed through alternative open reading frames and which are important in host defense suppression, virulence, and pathogenicity.

Influenza virus strains are classified according to host species of origin, geographic site and year of isolation, serial number, and, for influenza A virus (IAV), by serological properties of subtypes of HA and NA. For IAV, there are at least 16 HA subtypes (H1-H16) and nine NA subtypes (N1-N9) that have been identified. See, e.g., US Publ. No. 2007/0141078 A1; and Krammer et al., Nat Rev Dis Primers 4(1):3 (June 2018), each of which is incorporated herein by reference in its entirety. Unless indicated otherwise, the term “influenza,” as used herein, comprises all species, subtypes, and/or strains of the influenza virus.

As described herein, in some aspects, the ORF of a polynucleotide described herein encodes an influenza hemagglutinin (HA) protein, wherein the HA protein is not derived from H1N1pdm09_A/Korea/01/09. In some aspects, the ORF of a polynucleotide described herein encodes an influenza neuraminidase (NA) protein. In some aspects, the ORF encodes an influenza nucleoprotein (NP). In some aspects, the ORF encodes an influenza matrix 1 (M1) protein. In certain aspects, the ORF encodes an influenza matrix 2 (M2) protein. In some aspects, the ORF of a polynucleotide described herein encodes an influenza non-structural protein 1 (NS1) protein. In some aspects, the ORF of a polynucleotide described herein encodes an influenza non-structural protein 2 (NS2) protein. In certain aspects, the ORF encodes an influenza polymerase acidic (PA) protein. In some aspects, the ORF encodes an influenza polymerase basic 1 (PB1) protein. In some aspects, the ORF of a polynucleotide described herein encodes an influenza PB1-F2 protein. In some aspects, the ORF of a polynucleotide encodes an influenza polymerase basic 2 (PB2) protein.

In some aspects, the ORF of a polynucleotide described herein encodes a tumor antigen. Accordingly, in some aspects, a polynucleotide (e.g., isolated polynucleotide) of the present disclosure comprises: (i) an ORF encoding a tumor antigen, (ii) a 5′-untranslated region element (5′-UTR) of an influenza hemagglutinin (HA) protein (e.g., SEQ ID NO: 13), and (iii) a 3′-untranslated region element (3′-UTR) of an influenza hemagglutinin (HA) protein (e.g., SEQ ID NO: 14). As will be apparent from the present disclosure, any tumor antigen known in the art can be encoded using the ORFs of the polynucleotides described herein. Non-limiting examples of such tumor antigens include alpha-fetoprotein (AFP), B-cell maturation antigen (BCMA), carcinoembryonic antigen (CEA), epithelial tumor antigen (ETA), mucin 1 (MUC1), Tn-MUC1, mucin 16 (MUC16), tyrosinase, melanoma-associated antigen (MAGE; e.g., MAGEA3), tumor protein p53 (p53), CD4, CD8, CD45, CD80, CD86, programmed death ligand 1 (PD-L1), programmed death ligand 2 (PD-L2), prostate-specific membrane antigen (PSMA), TAG-72, human epidermal growth factor receptor 2 (HER2), GD2, cMET, EGFR, mesothelin, VEGFR, alpha-folate receptor, CE7R, IL-3, cancer-testis antigen (e.g., New York esophageal squamous cell carcinoma 1 (NY-ESO-1), MART-1 gp100, ROR1, ROR2, glypican-2, glypican-3, TNF-related apoptosis-inducing ligand, and combinations thereof. In some aspects, the tumor antigen is BCMA, In some aspects, the tumor antigen is NY-ESO-1. In some aspects, the tumor antigen is MAGEA3.

In some aspects, the ORF of a polynucleotide described herein encodes a protein associated with a genetic disorder. Not to be bound by any one theory, in some aspects, the ORF can encode a protein that is impaired in a subject having the genetic disorder, such that when a polynucleotide comprising the ORF is administered to the subject, the encoded protein can help restore the function associated with the impaired protein, and thereby, treat and/or reduce one or more symptoms associated with the genetic disorder. For instance, in some aspects, a genetic disorder that can be treated using the polynucleotides of the present disclosure comprise a Hunter syndrome.

As used herein, “Hunter syndrome” refers to a rare genetic disorder in which large sugar molecules called glycosaminoglycans (or GAGs or mucopolysaccharides) build up in various body tissues. The accumulation of GAG can cause wide-range of symptoms including, but not limited to, abnormal facial features (e.g., thickening of the lips, broad nose and flared nostrils, protruding tongue); enlarged head; deep and hoarse voice; abnormal bone size or shape and other skeletal irregularities; distended abdomen, as a result of enlarged organs; chronic diarrhea; white skin growths that resemble pebbles; joint stiffness; aggressive behavior; stunted growth; delayed development, such as late walking or talking; hearing loss; heart valve-related diseases; obstructive respiratory diseases; sleep apnea; and combinations thereof. Hunter syndrome is caused by a deficiency of the lysosomal enzyme iduronate-2-sulfatase (I2S). Accordingly, in some aspects, the ORF of a polynucleotide described herein encodes the I2S enzyme.

In some aspects, a polynucleotide described herein comprises a single ORF encoding a protein of interest. In certain aspects, a polynucleotide described comprises multiple ORFs (i.e., polycistronic polynucleotide). In some aspects, a polynucleotide described herein comprises two, three, four, or five or more ORFs. In certain aspects, one or more of the multiple ORFs encode a different protein of interest.

### II.C. Other Components

In some aspects, a polynucleotide of the present disclosure (e.g., comprising an ORF, HA-5′-UTR, and HA-3′-UTR) further comprises one or more additional components, which can help further increase the expression of the encoded protein. Non-limiting examples of such components are further described below.

### II.C.1. Cap

For instance, in certain aspects, a polynucleotide described herein comprises a 5′-cap. Accordingly, in some aspects, a polynucleotide of the present disclosure comprises (from 5′ to 3′): (i) a 5′-cap; (ii) HA-5′-UTR (e.g., SEQ ID NO: 13); (iii) an ORF encoding a protein of interest; and (iv) HA-3′-UTR (e.g., SEQ ID NO: 14).

As used herein, the term “5′-cap” refers to a modified nucleotide (e.g., guanine) that can be added to the 5′-end of a polynucleotide (e.g., mRNA). The 5′-cap structure can play a role in the nuclear export of the polynucleotide (e.g., to the cytoplasm where translation can occur) and/or promote the stability of the polynucleotide. In some aspects, the 5′-cap can be linked to the 5′-terminal end of the polynucleotide described herein via a 5′-5′-triphosphate linkage. In certain aspects, the 5′-cap can be methylated (e.g., m7GpppN, wherein N is the terminal 5′ nucleotide of the polynucleotide). Any suitable 5′-cap known in the art can be used with the present disclosure. Non-limiting examples of 5′-caps that can be used with the present disclosure include: m₂^{7,2′-O}GppspGRNA, m⁷GpppG, m⁷Gppppm⁷G, m₂^{(7,3′-O)}GpppG, m₂^{(7,2′-O)}GppspG(D1), m₂^{(7,2′-O)}GppspG(D2), m₂^{7,3′-O}Gppp(m₁^{2′-O})ApG, (m⁷G-3′ mppp-G; which may equivalently be designated 3′ O-Me-m7G(5′)ppp(5′)G), N7,2′-O-dimethyl-guanosine-5′-triphosphate-5′-guanosine, m⁷Gm-ppp-G, N7-(4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, or combinations thereof.

In some aspects, a 5′-cap that can be used with a polynucleotide of the present disclosure comprises a cap analog (also knowns as a “synthetic cap analog,” “chemical cap,” “chemical cap analog,” or “structural or functional cap analog”). “Cap analog” differs from natural (i.e., endogenous, wild-type, or physiological) 5′-caps in their chemical structure, while retaining cap function. Non-limiting examples of cap analogs are described in U.S. Pat. No. 8,519,110 and Kore et al., Bioorganic & Medicinal Chemistry 21:4570-4574 (2013), each of which is incorporated herein by reference in its entirety.

In some aspects, a 5′-cap is modified. A modification on the 5′-cap can further increase the stability, half-life, and/or translational efficiency of the polynucleotide. In some aspects, a modified 5′ cap comprises one or more of the following modifications: modification at the 2′ and/or 3′ position of a capped guanosine triphosphate (GTP), a replacement of the sugar ring oxygen (that produced the carbocyclic ring) with a methylene moiety (CH2), a modification at the triphosphate bridge moiety of the cap structure, or a modification at the nucleobase (G) moiety. See, e.g., US 2014/0147454 and WO 2018/160540, each of which is incorporated herein by reference in its entirety.

### II.C.2. Poly(A) Tail

In some aspects, a polynucleotide described herein (e.g., comprising an ORF, HA-5′-UTR, and HA-3′-UTR) further comprises a long chain of adenine nucleotides (referred to herein as “poly(A) tail”) at the 3′-end of the polynucleotide. In certain aspects, the poly(A) tail is present alone or in combination with other components described herein (e.g., 5′-cap). Accordingly, in some aspects, a polynucleotide described herein comprises (from 5′ to 3′): (i) a 5′-cap; (ii) HA-5′-UTR (e.g., SEQ ID NO: 13); (iii) an ORF encoding a protein of interest; (iv) HA-3′-UTR (e.g., SEQ ID NO: 14); and (v) a 3′-poly(A) tail.

In some aspects, the length of the poly(A) tail is greater than about 30 nucleotides in length. In certain aspects, the length of the poly(A) tail is at least about 35 nucleotides, at least about 40 nucleotides, at least about 45 nucleotides, at least about 50 nucleotides, at least about 60 nucleotides, at least about 70 nucleotides, at least about 80 nucleotides, at least about 90 nucleotides, at least about 100 nucleotides, at least about 110 nucleotides, at least about 120 nucleotides, at least about 130 nucleotides, at least about 140 nucleotides, at least about 150 nucleotides, at least about 160 nucleotides, at least about 170 nucleotides, at least about 180 nucleotides, at least about 190 nucleotides, at least about 200 nucleotides, at least about 250 nucleotides, at least about 300 nucleotides, at least about 350 nucleotides, at least about 400 nucleotides, at least about 450 nucleotides, or at least about 500 nucleotides or more.

Any suitable poly(A) tails known in the art can be used with the present disclosure. Non-limiting examples of poly(A) tails that can be used with the present disclosure include: SV40 poly(A), bGH poly(A), actin poly(A), hemoglobin poly(A), poly(A)-G quartet, or combinations thereof.

### II.C.3. Enhancers

In some aspects, the expression of a protein encoded by a polynucleotide described herein (e.g., comprising an ORF, HA-5′-UTR, and HA-3′-UTR) can be further increased using one or more enhancer sequences (also referred to herein as “translation enhancer element” or “TEE”). In some aspects, a polynucleotide described herein comprises at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, or at least about 50 or more enhancer sequences. Where a polynucleotide comprises multiple enhancers, in certain aspects, each of the enhancers is the same. In some aspects, one or more of the enhancers different. In some aspects, one or more of the enhancers are separated by a spacer.

Any enhancers known in the art can be used with the polynucleotides of the present disclosure. See, e.g., WO1999024595, WO2012009644, WO2009075886 and WO2007025008, European Patent Publication No. EP2610341A1 and EP2610340A1, U.S. Pat. Nos. 6,310,197, 6,849,405, 7,456,273, 7,183,395, each of which is herein incorporated by reference in its entirety. In some aspects, an enhancer useful for the present disclosure is a tissue-specific enhancer. In certain aspects, an enhancer that can be used with the present disclosure is selected from a human skeletal actin gene element, a cardiac actin gene element, a myocyte-specific enhancer binding factor MEF (e.g., MEF2), a MyoD enhancer element, a cardiac enhancer factor (CEF) site, murine creatine kinase enhancer element, skeletal fast-twitch troponin C gene element, slow-twitch cardiac troponin C gene element, the slow-twitch troponin I gene element, hypozia-inducible nuclear factors, steroid-inducible element, glucocorticoid response element (GRE), or any combination thereof.

II.C.4. microRNA Binding Site

In some aspects, the one or more additional components that can be present in a polynucleotide described herein (e.g., comprising an ORF, HA-5′-UTR, and HA-3′-UTR) comprises a microRNA (miRNA) binding site.

miRNAs are noncoding sequences that bind to the 3′-UTR of a polynucleotide and can down-regulate gene expression either by reducing the stability of the polynucleotide or by inhibiting translation. Not to be bound by any one theory, in some aspects, by engineering a miRNA binding site into a polynucleotide described herein, the expression of the encoded protein can be further regulated. For instance, if a polynucleotide described herein is not intended to target the liver, the polynucleotide can be modified to include a binding site for a miRNA that is abundant in the liver (e.g., miR-122), such that the encoded protein is not expressed in the liver when administered to a subject. Non-limiting examples of miRNAs that are abundantly expressed in different tissues include: liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

miRNAs are also known to be differentially expressed in immune cells. As used herein, “immune cells” include, but are not limited to, antigen presenting cells (APCs) (e.g., dendritic cells and macrophages), monocytes, B lymphocytes, T lymphocytes, granulocytes, and natural killer (NK) cells. In some aspects, by introducing a binding site for a miRNA that is abundantly expressed in certain immune cells, a polynucleotide of the present disclosure can be targeted to specific immune cells, and thereby, regulate a host immune response (e.g., by introducing binding sites for miR-142 and/or miR-146, which are abundantly expressed in myeloid dendritic cells, a polynucleotide of the present disclosure can be engineered to avoid targeting such cells).

Similarly, certain diseased cells/tissues (e.g., tumor cells or cells infected with a pathogen) have different miRNA expression profile (e.g., some are over-expressed while others are under-expressed) compared to healthy cells/tissues. See, e.g., US 2013/0053264 (prostate cancer); US 2011/0171646 (pancreatic cancer); U.S. Pat. No. 8,415,096 (asthma and inflammation); US 2012/0329672 (liver cancer); and US 2013/0053263 (pulmonary disease), each of which is incorporated herein by reference in its entirety. Accordingly, by introducing a binding site for certain miRNAs, polynucleotides of the present disclosure can also be engineered to specifically target certain diseased cells/tissues.

In some aspects, a polynucleotide described herein comprises at least one miRNA binding site. In certain aspects, a polynucleotide described herein comprises multiple miRNA binding sites. For instance, in some aspects, a polynucleotide comprises at least about 2, at least about 3, at least about 4, at least about 5, at least about 6, at least about 7, at least about 8, at least about 9, or at least about 10 or more miRNA binding sites. Where a polynucleotide comprises multiple miRNA binding sites, in some aspects, the multiple miRNA binding sites are the same. In certain aspects, one or more of the multiple miRNA binding sites are different (e.g., each of the multiple miRNA binding sites are specific for a different miRNA).

### II.C.5. IRES Sequences

In some aspects, a polynucleotide described herein (e.g., comprising an ORF, HA-5′-UTR, and HA-3′-UTR) can further comprise a nucleotide sequence encoding an internal ribosome-entry site (IRES). IRES plays an important role in initiating protein synthesis in absence of the 5′-cap structure. An IRES can act as the sole ribosome binding site, or can serve as one of multiple ribosome binding sites of a polynucleotide. Polynucleotides containing more than one functional ribosome binding site can encode several peptides or polypeptides that are translated independently by the ribosomes (“polycistronic polynucleotide”). Accordingly, where a polynucleotide described herein comprises a sequence encoding an IRES, in certain aspects, the polynucleotide can comprise multiple (e.g., at least two) translatable regions.

Any IRES sequences known in the art can be used with the present disclosure. Non-limiting examples of IRES sequences that can be used with the present disclosure include those from picornaviruses (e.g., FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV), cricket paralysis viruses (CrPV), or combinations thereof.

### II.C.6. Post-Transcriptional Regulatory Elements

In some aspects, an additional component that can be used with a polynucleotide described herein (e.g., comprising an ORF, HA-5′-UTR, and HA-3′-UTR) comprises a post-transcriptional regulatory element. In some aspects, the post-transcription regulatory element can be present in a polynucleotide described herein in combination with one or more other components described herein (e.g., 5′-cap, 3′-poly(A) tail, enhancer sequences, miRNA binding site, IRES sequence, or combinations thereof). In certain aspects, the post-translational regulatory element is positioned 3′- to the ORF of a polynucleotide described herein. Non-limiting examples of post-transcriptional regulatory elements that are useful for the present disclosure include a mutated woodchuck hepatitis virus post-transcriptional regulatory element (WPRE), microRNA binding site, DNA nuclear targeting sequence, or combinations thereof.

In some aspects, the one or more additional components that can be present in a polynucleotide described herein (e.g., comprising an ORF, HA-5′-UTR, and HA-3′-UTR) comprises a promoter. In certain aspects, a polynucleotide can include a single promoter. In some aspects, a polynucleotide can include multiple promoters (e.g., two, three, four, or five or more) that are operably linked to the ORF of a polynucleotide described herein. Where a polynucleotide comprises multiple promoters, in some aspects, each of the multiple promoters are the same. In certain aspects, one or more of the multiple promoters are different.

In some aspects, a promoter useful for the present disclosure comprises a mammalian promoter, viral promoter, or both. In certain aspects, a promoter that can be used with the polynucleotides described herein comprises a constitutive promoter, an inducible promoter, or both.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. As described herein, in some aspects, a promoter that can be used with the present disclosure is an inducible promoter. Inducible promoters are expressed in the presence of an inducing agent. For example, the metallothionein promoter is induced to promote transcription and translation in the presence of certain metal ions. In some aspects, a promoter that can be used comprises the T7 promoter.

### II.D. Modified Nucleosides/Nucleotides

In some aspects, a polynucleotide described herein (e.g., comprising an ORF, a HA-5′-UTR, and a HA-3′-UTR) comprises at least one chemically modified nucleoside and/or nucleotide. When the polynucleotides of the present disclosure are chemically modified, the polynucleotides can be referred to as “modified polynucleotides.”

A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside including a phosphate group. Modified nucleotides can be synthesized by any useful method, such as, for example, chemically, enzymatically, or recombinantly, to include one or more modified or non-natural nucleosides.

Polynucleotides can comprise a region or regions of linked nucleosides. Such regions can have variable backbone linkages. The linkages can be standard phosphodiester linkages, in which case the polynucleotides would comprise regions of nucleotides.

A polynucleotide of the present disclosure can comprise various distinct modifications. In some aspects, a polynucleotide can contain one, two, or more (optionally different) nucleoside or nucleotide modifications. In some aspects, a polynucleotide can exhibit one or more desirable properties, e.g., improved thermal or chemical stability, reduced immunogenicity, reduced degradation, increased binding to the target microRNA, reduced non-specific binding to other microRNA or other molecules, as compared to an unmodified polynucleotide.

In some aspects, a polynucleotide of the present disclosure is chemically modified. As used herein, in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population, including, but not limited to, its nucleobase, sugar, backbone, or any combination thereof.

In some aspects, a polynucleotide of the present disclosure can have a uniform chemical modification of all or any of the same nucleoside type or a population of modifications produced by downward titration of the same starting modification in all or any of the same nucleoside type, or a measured percent of a chemical modification of all any of the same nucleoside type but with random incorporation In further aspects, the polynucleotide of the present disclosure can have a uniform chemical modification of two, three, or four of the same nucleoside type throughout the entire polynucleotide (such as all uridines and/or all cytidines, etc. are modified in the same way).

Modified nucleotide base pairing encompasses not only the standard adenine-thymine, adenine-uracil, or guanine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleobase inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U” s. For example, TD's of the present disclosure can be administered as RNAs, as DNAs, or as hybrid molecules comprising both RNA and DNA units.

In some aspects, the polynucleotide described herein includes a combination of at least two (e.g., 2, 3, 4, 5, 6, 7, 8, 8, 10, 11, 12, 13, 14, 15, 16, 17, 18, 18, 20 or more) modified nucleobases.

In some aspects, the nucleobases, sugar, backbone linkages, or any combination thereof in a polynucleotide are modified by at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100%.

### II.D.1. Base Modification

In certain aspects, the chemical modification is at nucleobases in a polynucleotide of the present disclosure (e.g., comprising an ORF, HA-5′-UTR, and HA-3′-UTR). In some aspects, the at least one chemically modified nucleoside is a modified uridine (e.g., pseudouridine (ψ), 2-thiouridine (s2U), 1-methyl-pseudouridine (m1ψ), 1-ethyl-pseudouridine (e1ψ), or 5-methoxy-uridine (mo5U)), a modified cytosine (e.g., 5-methyl-cytidine (m5C)) a modified adenosine (e.g, 1-methyl-adenosine (m1A), N6-methyl-adenosine (m6A), or 2-methyl-adenine (m2A)), a modified guanosine (e.g., 7-methyl-guanosine (m7G) or 1-methyl-guanosine (m1G)), or a combination thereof.

In some aspects, the polynucleotide described herein is uniformly modified (e.g., fully modified, modified throughout the entire sequence) for a particular modification. For example, a polynucleotide can be uniformly modified with the same type of base modification, e.g., 5-methyl-cytidine (m5C), meaning that all cytosine residues in the polynucleotide sequence are replaced with 5-methyl-cytidine (m5C). Similarly, a polynucleotide can be uniformly modified for any type of nucleoside residue present in the sequence by replacement with a modified nucleoside such as any of those set forth above

In some aspects, the polynucleotide includes a combination of at least two (e.g., 2, 3, 4 or more) of modified nucleobases. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of a type of nucleobases in a polynucleotide of the present disclosure are modified nucleobases.

### II.D.2. Backbone Modification

In some aspects, a polynucleotide described herein can include any useful linkage between the nucleosides. Such linkages, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, —CH₂—O—N(CH₃)—CH₂—, —CH₂—N(CH₃)—N(CH₃)—CH₂—, —CH₂—NH—CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

In some aspects, the presence of a backbone linkage disclosed above increase the stability and resistance to degradation of a polynucleotide of the present disclosure. In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the backbone linkages in a polynucleotide of the present disclosure are modified (e.g., all of them are phosphorothioate).

In some aspects, a backbone modification that can be included in a polynucleotide of the present disclosure comprises phosphorodiamidate morpholino oligomer (PMO) and/or phosphorothioate (PS) modification.

### II.D.3. Sugar Modification

The modified nucleosides and nucleotides which can be incorporated into a polynucleotide of the present disclosure can be modified on the sugar of the nucleic acid. Incorporating affinity-enhancing nucleotide analogues, such as LNA or 2′-substituted sugars, can allow the length and/or the size of the polynucleotide to be modified (e.g., reduced).

In some aspects, at least about 5%, at least 10%, at least 15%, at least 20%, at least 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99% or 100% of the nucleotides in a polynucleotide of the present disclosure contain sugar modifications (e.g., LNA).

As described herein, in some aspects, a polynucleotide described herein can be a RNA (e.g., mRNA). Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

The 2′ hydroxyl group (OH) of ribose can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges include methylene, propylene, ether, amino bridges, aminoalkyl, aminoalkoxy, amino, and amino acid.

In some aspects, nucleotide analogues present in a polynucleotide of the present disclosure comprise, e.g., 2′-O-alkyl-RNA units, 2′-OMe-RNA units, 2′-O-alkyl-SNA, 2′-amino-DNA units, 2′-fluoro-DNA units, LNA units, arabino nucleic acid (ANA) units, 2′-fluoro-ANA units, HNA units, INA (intercalating nucleic acid) units, 2′MOE units, or any combination thereof. In some aspects, the LNA is, e.g., oxy-LNA (such as beta-D-oxy-LNA, or alpha-L-oxy-LNA), amino-LNA (such as beta-D-amino-LNA or alpha-L-amino-LNA), thio-LNA (such as beta-D-thio0-LNA or alpha-L-thio-LNA), ENA (such a beta-D-ENA or alpha-L-ENA), or any combination thereof. In further aspects, nucleotide analogues that can be included in a polynucleotide of the present disclosure comprises a locked nucleic acid (LNA), an unlocked nucleic acid (UNA), an arabino nucleic acid (ABA), a bridged nucleic acid (BNA), and/or a peptide nucleic acid (PNA).

In some aspects, a polynucleotide of the present disclosure can comprise both modified RNA nucleotide analogues (e.g., LNA) and DNA units. In some aspects, a miR-485 inhibitor is a gapmer. See, e.g., U.S. Pat. Nos. 8,404,649; 8,580,756; 8,163,708; 9,034,837; all of which are herein incorporated by reference in their entireties. In some aspects,

In some aspects, a polynucleotide of the present disclosure can include modifications to prevent rapid degradation by endo- and exo-nucleases. Modifications include, but are not limited to, for example, (a) end modifications, e.g., 5′ end modifications (phosphorylation, dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

### III. Vectors

In some aspects, provided herein are vectors (e.g., expression vectors) comprising a polynucleotide described herein (e.g., comprising an ORF, a HA-5′-UTR, and a HA-3′-UTR). As described herein, such vectors are useful for recombinant expression in host cells and cells targeted for therapeutic intervention. The term “vector,” as used herein, is intended to refer to a nucleic acid molecule capable of transporting another nucleic acid to which it has been linked; or an entity comprising such a nucleic acid molecule capable of transporting another nucleic acid. In some aspects, the vector is a “plasmid,” which refers to a circular double stranded DNA loop into which additional DNA segments can be ligated. In some aspects, the vector is a viral vector, wherein additional DNA segments can be ligated into the viral genome. Certain vectors, or polynucleotides that are part of vectors, are capable of autonomous replication in a host cell into which they are introduced (e.g., bacterial vectors having a bacterial origin of replication, and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present disclosure, “plasmid” and “vector” can sometimes be used interchangeably, depending on the context, as the plasmid is the most commonly used form of vector. However, also disclosed herein are other forms of expression vectors, such as viral vectors (e.g., lentiviruses, replication defective retroviruses, poxviruses, herpesviruses, baculoviruses, adenoviruses, and adeno-associated viruses), which serve equivalent functions

In some aspects, a vector comprises a polynucleotide described herein (e.g., comprising an ORF, a HA-5′-UTR, and a HA-3′-UTR) and a regulatory element. For instance, in certain aspects, a vector comprises a polynucleotide described herein (e.g., comprising an ORF, a HA-5′-UTR, and a HA-3′-UTR), operatively linked to a promoter. In some aspects, the vector can comprise a first ORF and a second ORF, wherein the first ORF is under the control of a first promoter and the second ORF is under the control of a second promoter. Where a polynucleotide described herein comprises multiple ORFs (e.g., two, three, four, or five or more), in certain aspects, each of the multiple promoters are the same. In some aspects, one or more of the multiple promoters are different.

Any suitable promoter known in the art can be used with the present disclosure. In some aspects, the promoters useful for the present disclosure comprises a mammalian or viral promoter, such as a constitutive or inducible promoter. In some aspects, the promoters for the present disclosure comprises at least one constitutive promoter and at least one inducible promoter, e.g., tissue specific promoter.

Constitutive mammalian promoters include, but are not limited to, the promoters for the following genes: hypoxanthine phosphoribosyl transferase (HPRT), adenosine deaminase, pyruvate kinase, beta-actin promoter, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus, and other retroviruses, and the thymidine kinase promoter of herpes simplex virus. As described herein, in some aspects, promoters that can be used with the present disclosure are inducible promoters. Inducible promoters are expressed in the presence of an inducing agent. For example, the metallothionein promoter is induced to promote transcription and translation in the presence of certain metal ions. When multiple inducible promoters are present, they can be induced by the same inducer molecule or a different inducer. In some aspects, a promoter that can be used with the polynucleotides of the present disclosure comprises the T7 promoter.

As further described elsewhere in the present disclosure, in some aspects, a vector comprising a polynucleotide described herein (e.g., comprising an ORF, a HA-5′-UTR, and a HA-3′-UTR) can additionally comprise one or more regulatory elements. Non-limiting examples of regulatory elements include a translation enhancer element (TEE), a translation initiation sequence, a microRNA binding site or seed thereof, a 3′ tailing region of linked nucleosides, an AU rich element (ARE), a post transcription control modulator, a 5′ UTR, a 3′ UTR, a localization sequence (e.g., membrane-localization sequences, nuclear localization sequences, nuclear exclusion sequences, or proteasomal targeting sequences), post-translational modification sequences (e.g., ubiquitination, phosphorylation, or dephosphorylation), or combinations thereof.

In some aspects, the vector can additionally comprise a transposable element. Accordingly, in certain aspects, the vector comprises a polynucleotide described herein (e.g., comprising an ORF, a HA-5′-UTR, and a HA-3′-UTR), which is flanked by at least two transposon-specific inverted terminal repeats (ITRs). In some aspects, the transposon-specific ITRs are recognized by a DNA transposon. In some aspects, the transposon-specific ITRs are recognized by a retrotransposon. Any transposon system known in the art can be used to introduce the nucleic acid molecules into the genome of a host cell, e.g., an immune cell. In some aspects, the transposon is selected from hAT-like Tol2, Sleeping Beauty (SB), Frog Prince, piggyBac (PB), and any combination thereof. In some aspects, the transposon comprises Sleeping Beauty. In some aspects, the transposon comprises piggyBac. See, e.g., Zhao et al., Transl. Lung Cancer Res. 5(1):120-25 (2016), which is incorporated by reference herein in its entirety.

In some aspects, the vector is a transfer vector. The term “transfer vector” refers to a composition of matter which comprises an isolated nucleic acid (e.g., a polynucleotide of the present disclosure) and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

In some aspects, the vector is an expression vector. The term “expression vector” refers to a vector comprising a recombinant polynucleotide (e.g., a polypeptide of the present disclosure) comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some aspects, the vector is a viral vector, a mammalian vector, or bacterial vector. In some aspects, the vector is selected from the group consisting of an adenoviral vector, a lentivirus, a Sendai virus vector, a baculoviral vector, an Epstein Barr viral vector, a papovaviral vector, a vaccinia viral vector, a herpes simplex viral vector, a hybrid vector, and an adeno associated virus (AAV) vector.

In some aspects, non-viral methods can be used to deliver a polynucleotide of the present disclosure (e.g., comprising an ORF, a HA-5′-UTR, and a HA-3′-UTR) into a cell or tissue of a subject. In some aspects, the non-viral method includes the use of a transposon. In some aspects, use of a non-viral method of delivery permits reprogramming of cells, e.g., T or NK cells, and direct infusion of the cells into the subject. In some aspects, a polynucleotide of the present disclosure (e.g., comprising an ORF, a HA-5′-UTR, and a HA-3′-UTR) can be inserted into the genome of a target cell (e.g., a T cell) or a host cell (e.g., a cell for recombinant expression of the encoded proteins) by using CRISPR/Cas systems and genome edition alternatives, such as zinc-finger nucleases (ZFNs), transcription activator-like effector nucleases (TALENs), and meganucleases (MNs).

### IV. Delivery Agents

In some aspects, a polynucleotide of the present disclosure can be administered (e.g., to a subject in need thereof) with a delivery agent. Non-limiting examples of delivery agents that can be used include a lipidoid, a liposome, a lipoplex, a lipid nanoparticle, a polymeric compound, a peptide, a protein, a cell, a nanoparticle mimic, a nanotube, a micelle, a conjugate, and combinations thereof.

Thus, in some aspects, the present disclosure also provides a composition comprising a polynucleotide described herein (e.g., comprising an ORF, a HA-5′-UTR, and a HA-3′-UTR) and a delivery agent. In some aspects, the delivery agent comprises a cationic carrier unit comprising

[WP]-L1-[CC]-L2-[AM] (formula I)

or

[WP]-L1-[AM]-L2-[CC] (formula II)

wherein
WP is a water-soluble biopolymer moiety;
CC is a positively charged carrier moiety;
AM is an adjuvant moiety; and,
L1 and L2 are independently optional linkers, and
wherein when mixed with a nucleic acid at an ionic ratio of about 1:1, the cationic carrier unit forms a micelle.

In some aspects, composition comprising a polynucleotide described herein interacts with the cationic carrier unit via an ionic bond.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble polymer comprises polyethylene glycol (“PEG”), polyglycerol, or poly(propylene glycol) (“PPG”) In some aspects, the water-soluble polymer comprises:

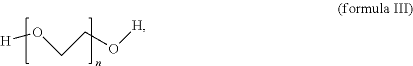

wherein n is 1-1000.

In some aspects, the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141. In some aspects, the n is about 80 to about 90, about 90 to about 100, about 100 to about 110, about 110 to about 120, about 120 to about 130, about 140 to about 150, about 150 to about 160. In certain aspects, the n is about 114.

In some aspects, the water-soluble polymer is linear, branched, or dendritic. In some aspects, the cationic carrier moiety comprises one or more basic amino acids. In some aspects, the cationic carrier moiety comprises at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 110, at least about 120, at least about 130, at least about 140 basic amino acids. In some aspects, the cationic carrier moiety comprises about 30 to about 50 basic amino acids. In some aspects, the cationic carrier moiety comprises about 60, about 70, about 80, about 90, or about 100 basic amino acids. In certain aspects, the cationic carrier moiety comprises about 80 basic amino acids. In some aspects, the basic amino acid comprises arginine, lysine, histidine, or any combination thereof. In some aspects, the cationic carrier moiety comprises about 40 lysine monomers.

In some aspects, the adjuvant moiety is capable of modulating an immune response, an inflammatory response, and/or a tissue microenvironment. In some aspects, the adjuvant moiety comprises an imidazole derivative, an amino acid, a vitamin, or any combination thereof. In some aspects, the adjuvant moiety comprises:

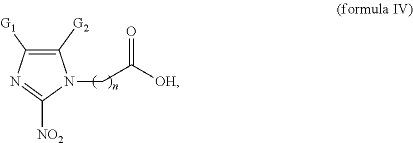

wherein each of G1 and G2 is H, an aromatic ring, or 1-10 alkyl, or G1 and G2 together form an aromatic ring, and wherein n is 1-10.

In some aspects, the adjuvant moiety comprises nitroimidazole. In some aspects, the adjuvant moiety comprises metronidazole, tinidazole, nimorazole, dimetridazole, pretomanid, ornidazole, megazol, azanidazole, benznidazole, or any combination thereof. In some aspects, the adjuvant moiety comprises an amino acid.

In some aspects, the adjuvant moiety comprises

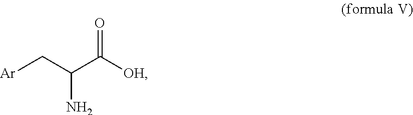

wherein Ar is

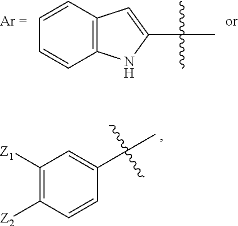

and
wherein each of Z1 and Z2 is H or OH.

In some aspects, the adjuvant moiety comprises a vitamin. In some aspects, the vitamin comprises a cyclic ring or cyclic hetero atom ring and a carboxyl group or hydroxyl group. In some aspects, the vitamin comprises:

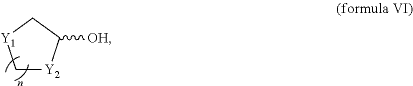

wherein each of Y1 and Y2 is C, N, O, or S, and wherein n is 1 or 2.

In some aspects, the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof. In some aspects, the vitamin is vitamin B3.

In some aspects, the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 vitamin B3. In some aspects, the adjuvant moiety comprises about 35 vitamin B3.

In some aspects, the composition comprises a water-soluble biopolymer moiety with about 100 to about 130 PEG units, a cationic carrier moiety comprising a poly-lysine with about 30 to about 100 lysines (e.g., about 80 lysines), and an adjuvant moiety with about 5 to about 50 vitamin B3 (e.g., about 35 vitamin B3).

In some aspects, the composition comprises (i) a water-soluble biopolymer moiety with about 100 to about 200 PEG units, (ii) about 30 to about 100 lysines with an amine group (e.g., about 40 lysines), (iii) about 1 to 20 lysines, each having a thiol group (e.g., about 5 lysines, each with a thiol group), and (iv) about 5 to 50 lysines fused to vitamin B3 (e.g., about 35 lysines, each fused to vitamin B3). In some aspects, the composition further comprises a targeting moiety, e.g., a LAT1 targeting ligand, e.g., phenyl alanine, linked to the water soluble polymer. In some aspects, the thiol groups in the composition form disulfide bonds.

In some aspects, the composition comprises (1) a micelle comprising (i) about 100 to about 200 PEG units (e.g., about 114 units), (ii) about 30 to about 100 lysines with an amine group (e.g., about 40 lysines), (iii) about 3 to 50 lysines, each having a thiol group (e.g., about 35 lysines, each with a thiol group), and (iv) about 2 to 20 lysines fused to vitamin B3 (e.g., about 5 lysines, each fused to vitamin B3), and (2) an isolated polynucleotide described herein (e.g., comprising an ORF, a HA-5′-UTR, and a HA-3′-UTR), wherein the isolated polynucleotide is encapsulated within the micelle. In some aspects, the composition further comprises a targeting moiety, e.g., a LAT1 targeting ligand, e.g., phenyl alanine, linked to the PEG units. In some aspects, the thiol groups in the micelle form disulfide bonds.

The present disclosure also provides a micelle comprising a polynucleotide described herein (e.g., comprising an ORF, a HA-5′-UTR, and a HA-3′-UTR), wherein the polynucleotide and the delivery agent are associated with each other.

In some aspects, the association is a covalent bond, a non-covalent bond, or an ionic bond. In some aspects, the positive charge of the cationic carrier moiety of the cationic carrier unit is sufficient to form a micelle when mixed with the polynucleotide disclosed herein in a solution, wherein the overall ionic ratio of the positive charges of the cationic carrier moiety of the cationic carrier unit and the negative charges of the polynucleotide (or vector comprising the polynucleotide) in the solution is about 1:1.

In some aspects, the cationic carrier unit is capable of protecting the polynucleotide of the present disclosure from enzymatic degradation. See PCT Publication No. WO 2020/261227, published Dec. 30, 2020, which is herein incorporated by reference in its entirety.

In some aspects, the polynucleotides disclosed herein (e.g., comprising an ORF, a HA-5′-UTR, and a HA-3′-UTR) are DNA (e.g., a DNA molecule or a combination thereof), RNA (e.g., a RNA molecule or a combination thereof), or any combination thereof. In some aspects, the polynucleotides described herein comprise nucleic acid sequences comprising single stranded or double stranded RNA or DNA (e.g., ssDNA or dsDNA) in genomic or cDNA form, or DNA-RNA hybrids, each of which can include chemically or biochemically modified, non-natural, or derivatized nucleotide bases. As described herein, such nucleic acid sequences can comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals.

### IV.A. Carrier Units

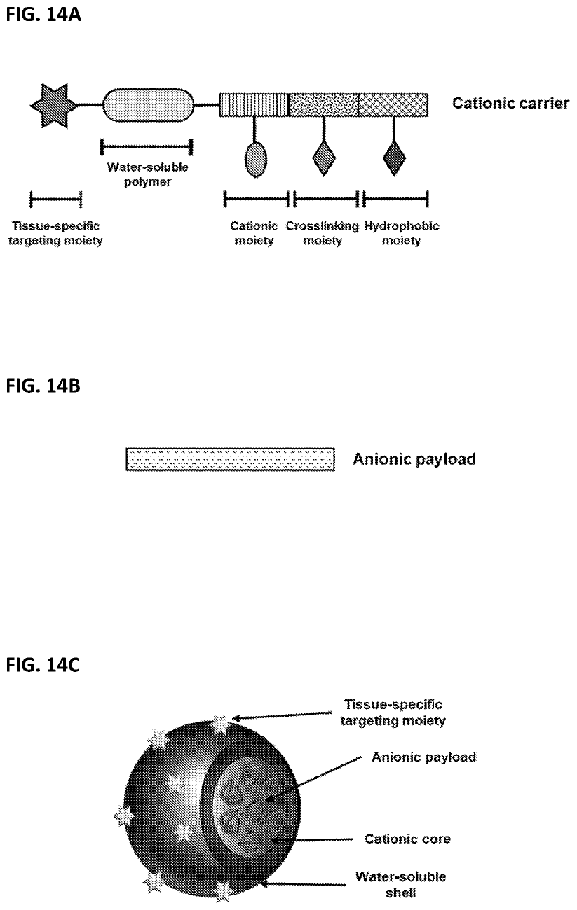

The present disclosure provides carrier units that can self-assemble into micelles or be incorporated into micelles. In some aspects, carrier units of the present disclosure comprise a water-soluble biopolymer moiety (e.g., PEG), a charged carrier moiety, a crosslinking moiety, and a hydrophobic moiety. In some aspects, the charged carrier moiety is cationic (e.g., a polylysine) (see, e.g., FIG. 14A).

Carrier units of the present disclosure can be used to deliver negatively charged payloads (e.g., polynucleotides disclosed herein). In some aspects, negatively charged payloads (e.g., polynucleotides disclosed herein) that can be delivered by micelles comprise at least about 100, at least about 200, at least about 300, at least about 400, at least about 500, at least about 600, at least about 700, at least about 800, at least about 900, at least about 1000, at least about 1100, at least about 1200, at least about 1300, at least about 1400, at least about 1500, at least about 1600, at least about 1700, at least about 1800, at least about 1900, at least about 2000, at least about 2100, at least about 2200, at least about 2300, at least about 2400, at least about 2500, at least about 2600, at least about 2700, at least about 2800, at least about 2900, at least about 3000, at least about 3100, at least about 3200, at least about 3300, at least about 3400, at least about 3500, at least about 3600, at least about 3700, at least about 3800, at least about 3900, or at least about 4000 nucleotides in length. Carrier units with a cationic charged carrier moiety can be used for the delivery of anionic payloads, e.g., polynucleotides. Carrier units with an anionic charged carrier moiety can be used for the delivery of cationic payloads, e.g., positively charged polynucleotides. In some aspects, the cationic charged carrier moiety and the anionic payloads can electrostatically interact with each other.

The resulting carrier unit: payload complex can have a “head” comprising the water-soluble biopolymer moiety and a “tail” comprising the cationic carrier moiety electrostatically bound to the payload.

Carrier unit: payload complexes can self-associate, alone or in combination with other molecules, to yield micelles in which the anionic payload (e.g., polynucleotides disclosed herein) is located in the core of the micelle and the water-soluble biopolymer moiety is facing the solvent. The term “micelles of the present disclosure” encompasses not only classic micelles but also small particles, small micelles, micelles, rod-like structures, or polymersomes. Given that polymersomes comprise a luminal space, it is to be understood that all the disclosures related to the “core” of classic micelles are equally applicable to the luminal space in polymersomes comprising carrier units of the present disclosure.

The carrier units of the present disclosure can also comprise a targeting moiety (e.g., a targeting ligand) covalently linked to the water-soluble biopolymer moiety via one or more optional linkers. Once a micelle is formed, the targeting moiety can be located on the surface of the micelle and can deliver the micelle to a specific target tissue, to a specific cell type, and/or facilitate transport across a physiological barrier (e.g., cell plasma membrane). In some aspects, the micelles of the present disclosure can comprises more than one type of targeting moiety.

The carrier units of the present disclosure can also comprise a hydrophobic moiety (HM) covalently linked to the charged cationic carrier moiety. The hydrophobic moiety can have, e.g., a therapeutic, a co-therapeutic effect, or positively affect the homeostasis of the target cell or target tissue. In some aspects, the HM comprises one or more amino acids. In some aspects, the HM comprises one or more amino acids linked to a hydrophobic molecule (e.g., a vitamin). In some aspects, the HM comprises one or more lysine residues covalently bound to a hydrophobic molecule (e.g., a vitamin).

In some aspects, the anionic payload (e.g., polynucleotides disclosed herein) is not covalently linked to the carrier unit. In certain aspects, the anionic payload (e.g., polynucleotides disclosed herein) can be covalently linked to the cationic carrier unit, e.g., a linker such as cleavable linker.

Non-limiting examples of various aspects are shown in the present disclosure. The disclosure refers in particular to the use of cationic carrier units, e.g., to deliver anionic payloads such as nucleic acids. However, it would be apparent to a person of ordinary skill in the art that the disclosures can be equally applied to the delivery of cationic payloads or to the delivery of neutral payloads by reversing the charges of the carrier moiety and payload (i.e., using an anionic carrier moiety in the carrier unit to deliver a cationic payload), or by using a neutral payload linked to a cationic or anionic adapter that would electrostatically interact with an anionic or cationic carrier moiety, respectively.

Accordingly, in one aspect, the present disclosure provides cationic carrier units of Schema I through Schema VI

[CC]-L1-[CM]-L2-[HM] (Schema I);

[CC]-L1-[HM]-L2-[CM] (Schema II);

[HM]-L1-[CM]-L2-[CC] (Schema III);

[HM]-L1-[CC]-L2-[CM] (Schema IV);

[CM]-L1-[CC]-L2-[HM] (Schema V); or

[CM]-L1-[HM]-L2-[CC] (Schema VI);
wherein
CC is a cationic carrier moiety, e.g., a polylysine;
CM is a crosslinking moiety;
HM is a hydrophobic moiety, e.g., vitamin, e.g., vitamin B3; and,
L1 and L2 are independently optional linkers.

In some aspects, the cationic carrier unit further comprises a water-soluble polymer (WP). In some aspects, the water-soluble polymer is attached to [CC], [HM], and/or [CM]. In some aspects, the water-soluble polymer is attached to the N terminus of [CC], [HM], or [CM]. In some aspects, the water-soluble polymer is attached to the N terminus of [CC]. In some aspects, the water-soluble polymer is attached to the C terminus of [CC], [HM], or [CM]. In some aspects, the water-soluble polymer is attached to the C terminus of [CC].

In some aspects, the cationic carrier unit comprises:

[WP]-L3-[CC]-L1-[CM]-L2-[HM] (Schema I′);

[WP]-L3-[CC]-L1-[HM]-L2-[CM] (Schema II′);

[WP]-L3-[HM]-L1-[CM]-L2-[CC] (Schema III′);

[WP]-L3-[HM]-L1-[CC]-L2-[CM] (Schema IV′);

[WP]-L3-[CM]-L1-[CC]-L2-[HM] (Schema V′); or

[WP]-L3-[CM]-L1-[HM]-L2-[CC] (Schema VI′).

In some aspects of the constructs of Schema I′ to VI′ shown above, the [WP] component can be connected to at least one targeting moiety, i.e., [T]ₙ-[WP]- . . . wherein n is an integer, e.g., 1, 2 or 3.

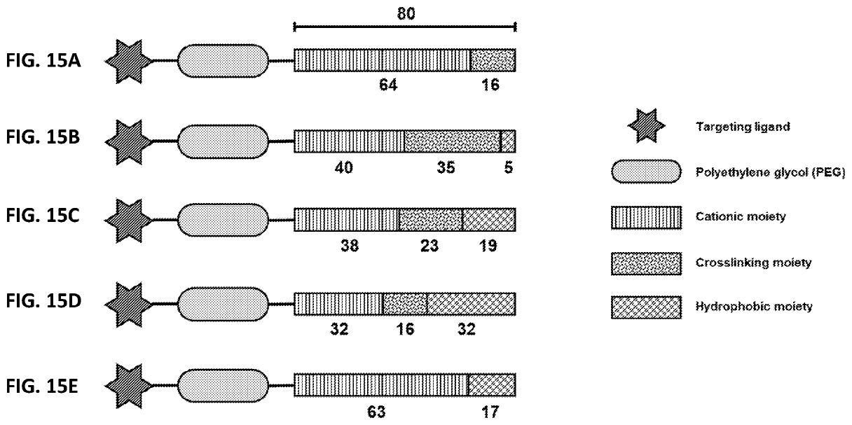

FIGS. 15A-15E present a schematic representation of a cationic carrier unit of the present disclosure. For simplicity, the units in FIG. 15A-15E have been represented linearly. However, in some aspects, the carrier units can comprises the CC, CM, and HM moieties organized in a branched scaffold arrangement (see FIG. 15A and FIG. 14E), for example, with (i) a polymeric CC moiety comprising positively charged units (e.g., polylysines) and (ii) a CMs (e.g., lysine linked to a crosslinking agent, e.g., lysine-thiol) attached to the N or C terminus of the CC moeity and (iii) a HM (e.g., lysine linked to a hydrophobic agent, e.g., lysine linked to Vitamin B3) attached to the N or C terminus of the CM moiety.

In some aspects, the carrier units can comprises the CC, CM, and HM moieties organized in a branched scaffold arrangement, for example, with (i) a polymeric CC moiety comprising positively charged units (e.g., polylysines) and (ii) a CMs (e.g., lysine linked to a crosslinking agent, e.g., lysine-thiol) attached to the N or C terminus of the CC moeity and (iii) a HM (e.g., lysine linked to a hydrophobic agent, e.g., lysine linked to Vitamin B3) attached to the N or C terminus of the CM moiety.

When cationic carrier units of the present disclosure are mixed with an anionic payload (e.g., a nucleic acid) at an ionic ratio of about 20:about 1, i.e., the number of negative charges in the anionic payload is about 20 times higher than the number of positive charges in the cationic carrier moiety, to about 20:1, i.e., the number of positive charges in the cationic carrier moiety is about ten times higher than the number of negative charges in the anionic payload, the neutralization of negative charges in the anionic payload by positive charges in the cationic carrier moiety mainly via electrostatic interaction leads to the formation of a cationic carrier unit: anionic payload complex having an unaltered hydrophilic portion (comprising the WP moiety) and a substantially more hydrophobic portion (resulting from the association between the cationic carrier moiety plus hydrophobic moiety and the anionic payload).

In some aspects, the hydrophobic moiety can contribute its own positive charges to the positive charges of the cationic carrier moiety, which would interact with the negative charges of the anionic payload (e.g., polynucleotides disclosed herein). It is to be understood that references to the interactions (e.g., electrostatic interactions) between a cationic carrier moiety and an anionic payload (e.g., polynucleotides disclosed herein) also encompass interactions between the charges of a cationic carrier moiety plus hydrophobic moiety and the charges of an anionic payload.

The increase in the hydrophobicity of the cationic carrier moiety of the cationic carrier unit due to the neutralization of its positive charges via electrostatic interaction with the negative charges of the anionic payload results in an amphipathic complex. Such amphipathic complexes can self-organize, alone or combination with other amphipathic components, into micelles. The resulting micelles comprise the WP moieties facing the solvent (i.e., the WP moieties are facing the external surface of the micelle), whereas the CC and HM moieties as well as the associate payload (e.g., a nucleotide sequence, e.g., RNA, DNA, or any combination thereof) are in the core of the micelle.

In some specific aspects, the cationic carrier unit comprises:
(a) a WP moiety, wherein the water-soluble biopolymer is a polyethylene glycol (PEG) of formula III (see below), wherein n is between about 120 to about PEG 130 (e.g., PEG is a PEG5000 or a PEG6000);
(b) a CC moiety, wherein the cationic carrier moiety comprises, e.g., about 20 to about 100 lysines (e.g., a linear poly(L-lysine)n wherein n is between about 30 and about 40), a polyethyleneimine (PEI), or chitosan;
(c) a CM moiety, wherein the crosslinking moiety comprises about about 10 to about 50 lysines, each of which is linked to a crosslinking agent, e.g., 10-40 lysine-thiol, and
(d) an HM moiety, wherein the hydrophobic moiety has about 1 to about 20 lysines, each of which is linked to a vitamin B3 unit.

In some specific aspects, the cationic carrier unit comprises:
(a) a WP moiety, wherein the water-soluble biopolymer is a polyethylene glycol (PEG) of formula III (see below), wherein n is between about 120 to about PEG 130 (e.g., PEG is a PEG5000 or a PEG6000);
(b) a CC moiety, wherein the cationic carrier moiety comprises, e.g., about 20 to about 100 lysines (e.g., a linear poly(L-lysine)n wherein n is between about 30 and about 40), a polyethyleneimine (PEI), or chitosan;
(c) a CM moiety, wherein the crosslinking moiety comprises about about 10 to about 50 lysines, each of which is linked to a crosslinking agent, e.g., 10-40 lysine-thiol, and
(d) an HM moiety, wherein the hydrophobic moiety has about 1 to about 10 lysines, each of which is linked to a vitamin B3 unit.

In some specific aspects, the cationic carrier unit comprises:
(a) a WP moiety, wherein the water-soluble biopolymer is a polyethylene glycol (PEG) of formula III (see below), wherein n is between about 120 to about PEG 130 (e.g., PEG is a PEG5000 or a PEG6000);
(b) a CC moiety, wherein the cationic carrier moiety comprises, e.g., about 20 to about 100 lysines (e.g., a linear poly(L-lysine)n wherein n is between about 30 and about 40), a polyethyleneimine (PEI), or chitosan;
(c) a CM moiety, wherein the crosslinking moiety comprises about about 10 to about 50 lysines, each of which is linked to a crosslinking agent, e.g., 10-40 lysine-thiol, and
(d) an HM moiety, wherein the hydrophobic moiety has about 5 to about 10 lysines, each of which is linked to a vitamin B3 unit.

In some specific aspects, the cationic carrier unit comprises:
(a) a WP moiety, wherein the water-soluble biopolymer is a polyethylene glycol (PEG) of formula III (see below), wherein n is between about 120 to about PEG 130 (e.g., PEG is a PEG5000 or a PEG6000);
(b) a CC moiety, wherein the cationic carrier moiety comprises, e.g., about 20 to about 100 lysines (e.g., a linear poly(L-lysine)n, wherein n is between about 30 and about 40, e.g., about 40), a polyethyleneimine (PEI), or chitosan;
(c) a CM moiety, wherein the crosslinking moiety comprises about about 10 to about 50 lysines, each of which is linked to a crosslinking agent, e.g., 10-40 lysine-thiol, e.g., 35 lysine-thiol, and
(d) an HM moiety, wherein the hydrophobic moiety has about 1 to about 5 lysines (e.g., about 5), each of which is linked to a vitamin B3 unit.

In some aspects, the cationic carrier unit further comprises at least one targeting moiety attached to the WP moiety of the cationic carrier unit. In some aspects, the number and/or density of targeting moieties displayed on the surface of the micelle can be modulated by using a specific ratio of cationic carrier units having targeting moieties to cationic carrier units not having targeting moieties. In some aspects, the ratio of cationic carrier units having a targeting moiety to cationic carrier units not having a targeting moiety is at least about 1:5, at least about 1:10, at least about 1:20, at least about 1:30, at least about 1:40, at least about 1:50, at least about 1:60, at least about 1:70, at least about 1:80, at least about 1:90, at least about 1:100, at least about 1:120, at least about 1:140, at least about 1:160, at least about 1:180, at least about 1:200, at least about 1:250, at least about 1:300, at least about 1:350, at least about 1:400, at least about 1:450, at least about 1:500, at least about 1:600, at least about 1:700, at least about 1:800, at least about 1:900, or at least about 1:1000.

In some aspects, the cationic carrier unit comprises
(i) a targeting moiety (A) which targets the transporter LAT1 (e.g., phenylalanine),
(ii) a water soluble polymer which is PEG,
(iii) a cationic carrier moiety comprising cationic polymer blocks which are lysine
(iv) a crosslinking moiety comprising crosslinking polymer blocks which are lysines linked to crosslinking moieties, and
(v) a hydrophobic moiety comprising hydrophobic polymer blocks which are lysines linked to vitamin B3.

In some aspects, the cationic carrier unit comprises
(i) a targeting moiety (A) which targets the transporter LAT1 (e.g., phenylalanine),
(ii) a water soluble polymer which is PEG, wherein n=100-200, e.g., 100-150, e.g., 120-130,
(iii) a cationic carrier moiety comprising cationic polymer blocks, e.g., polylysine,
(iv) a crosslinking moiety comprising crosslinking polymer blocks which are lysines linked to crosslinking moieties, and
(iv) a hydrophobic moiety comprising hydrophobic polymer blocks which are lysines linked to vitamin B3.

In some aspects, the cationic carrier unit comprises
(i) a targeting moiety (A) which targets the transporter LAT1 (e.g., phenylalanine),
(ii) a water soluble polymer which is PEG, wherein n=100-200, e.g., 100-150, e.g., 120-130,
(iii) a cationic carrier moiety comprising cationic polymer blocks, e.g., 10-100 lysines, e.g., 10-50 lysines, e.g., 30-40 lysines,
(iv) a crosslinking moiety comprising crosslinking polymer blocks which are lysines linked to crosslinking moieties, and
(iv) a hydrophobic moiety comprising hydrophobic polymer blocks which are lysines linked to vitamin B3.

In some aspects, the number (percentage) of HM is less than 39%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or about 1% relative to [CC] and [CM]. In some aspects, the number (percentage) of HM is between about 35% and about 1%, about 35% and about 5%, about 35% and about 10%, about 35% and about 15%, about 35% and about 20%, about 35% and about 25%, about 35% and about 30%, about 30% and about 1%, about 30% and about 5%, about 30% and about 10%, about 30% and about 15%, about 30% and about 20%, about 30% and about 25%, about 25% and about 1%, about 25% and about 5%, about 25% and about 10%, about 25% and about 15%, about 25% and about 20%, about 20% and about 1%, about 20% and about 5%, about 20% and about 10%, about 20% and about 15%, about 15% and about 1%, about 15% and about 5%, about 15% and about 10%, about 10% and about 1%, or about 10% and about 5% relative to [CC] and [CM]. In some aspects, the number (percentage) of HM is between about 39% and about 30%, about 30% and about 20%, about 20% and about 10%, about 10% and about 5%, and about 5% and about 1% relative to [CC] and [CM]. In some aspects, the number (percentage) of HM is about 39%, about 30%, about 25%, about 20%, about 15%, about 10%, about 5%, or about 1% relative to [CC] and [CM]. In some aspects, the the number of HM is expressed as the percentage of [HM] relative to [CC] and [CM].

In some aspects, the cationic carrier unit of the present disclosure interacts with a nucleotide payload having about 100 to about 4000 nucleotides in length. In some aspects, the nucleotide payload having about 100 to about 4000 nucleotides in length encodes one or more proteins or fragments thereof, e.g., coronavirus (e.g., SARS-CoV-2) spike protein, or any fragments thereof.

In some aspects, the vitamin B3 unit are introduced into the side chains of the HM moiety, e.g., by a coupling reaction between NH₂ groups in the lysines and COOH groups of vitamin B3, in the presence of suitable conjugation reagents, for example, 1-ethyl-3-(3-dimethylaminopropyl)-carbodiimide (EDC) and N-hydroxy succinimide (NHS).

The present disclosure provides composition comprising a carrier unit (e.g., a cationic carrier unit) of the present disclosure. In other aspects, the present disclosure provides complexes comprising a carrier unit (e.g., a cationic carrier unit unit) of the present disclosure non-covalently attached to a payload (e.g., an anionic payload such a nucleotide sequence, e.g., an RNA, DNA, or any combination thereof), wherein the carrier unit and the payload interact electrostatically. In other aspects, the present disclosure provides conjugates comprising a carrier unit (e.g., a cationic carrier unit unit) of the present disclosure covalently attached to a payload (e.g., an anionic payload such a nucleotide sequence, e.g., an RNA, DNA, or any combination thereof), wherein the carrier unit and the payload interact electrostatically. In some aspects, the carrier unit and the payload can be linked via a cleavable linker. In some aspects, the carrier unit and the payload, in addition to interacting electrostatically, can interact covalently (e.g., after electrostatic interaction the carrier unit and the payload can be “locked” via a disulfide bond or a cleavable bond).

In some specific aspects, the cationic carrier unit comprises a water-soluble polymer comprising a PEG with about 120 to about 130 units, a cationic carrier moiety comprising a polylysine with about 20 to about 60 lysine units, (e.g., about 40 lysines) a crosslinking moiety comprising about 3 to about 40 lysine-thiol units (e.g., about 5 lysines, each with a thiol group), and a hydrophobic moiety comprising about 1 to about 50 lysines linked to a vitamin B3 units (e.g., about 35 lysines, each fused to vitamin B3).

In some aspects, the cationic carrier unit is associated with a negatively charged payload (e.g., a nucleotide sequence, e.g., an RNA, DNA, or any combination thereof), which interacts with the cationic carrier unit via at least one ionic bond (i.e., via electrostatic interaction) with the cationic carrier moiety of the cationic carrier unit.

The specific components of the cationic carrier units of the present disclosure are disclosed in detail below.

### IV.A.1. Water-Soluble Biopolymer

In some aspects, the cationic carrier units of the present disclosure comprise at least one water-soluble biopolymer. The term “water-soluble biopolymer” as used herein refers to a biocompatible, biologically inert, non-immunogenic, non-toxic, and hydrophilic polymer, e.g., PEG.

In some aspects, the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof. In some aspects, the water-soluble biopolymer is linear, branched, or dendritic.

In some aspects, the water-soluble biopolymer comprises polyethylene glycol (“PEG”), polyglycerol (“PG”), or poly(propylene glycol) (“PPG”). PPG is less toxic than PEG, so many biological products are now produced in PPG instead of PEG.

In some aspects, the water-soluble biopolymer comprises a PEG characterized by a formula R³—(O—CH₂—CH₂)ₙ— or R³—(O—CH₂—CH₂)ₙ—O— with R³ being hydrogen, methyl or ethyl and n having a value from 2 to 200. In some aspects, the PEG has the formula

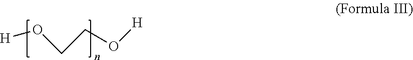

wherein n is 1 to 1000.

In some aspects, the n of the PEG has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n is at least about 10, at least about 20, at least about 30, at least about 40, at least about 50, at least about 60, at least about 70, at least about 80, at least about 90, at least about 100, at least about 110, at least 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 670, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, or about 1000.

In some aspects, n is between about 50 and about 100, between about 100 and about 150, between about 150 and about 200, between about 200 and about 250, between about 250 and about 300, between about 300 and about 350, between about 350 and about 400, between about 400 and about 450, between about 450 and about 500, between about 500 and about 550, between about 550 and about 600, between about 600 and about 650, between about 650 and about 700, between about 700 and about 750, between about 750 and about 800, between about 800 and about 850, between about 850 and about 900, between about 900 and about 950, or between about 950 and about 1000.

In some aspects, n is at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, or at least about 160.

In some aspects, n is about 80 to about 90, about 90 to about 100, about 100 to about 110, about 110 to about 120, about 120 to about 130, about 130 to about 140, about 140 to about 150, about 150 to about 160, about 85 to about 95, about 95 to about 105, about 105 to about 115, about 115 to about 125, about 125 to about 135, about 135 to about 145, about 145 to about 155, about 155 to about 165, about 80 to about 100, about 100 to about 120, about 120 to about 140, about 140 to about 160, about 85 to about 105, about 105 to about 125, about 125 to about 145, or about 145 to about 165.

In some aspects, n is about 100 to about 150. In some aspects, n is about 100 to about 140. In some aspects, n is about 100 to about 130. In some aspects, n is about 110 to about 150. In some aspects, n is about 110 to about 140. In some aspects, n is about 110 to about 130. In some aspects, n is about 110 to about 120. In some aspects, n is about 120 to about 150. In some aspects, n is about 120 to about 140. In some aspects, n is about 120 to about 130. In some aspects, n is about 130 to about 150. In some aspects, n is about 130 to about 140. In some aspects, n is about 114.

Thus, is some aspects, the PEG is a branched PEG. Branched PEGs have three to ten PEG chains emanating from a central core group. In certain aspects, the PEG moiety is a monodisperse polyethylene glycol. In the context of the present disclosure, a monodisperse polyethylene glycol (mdPEG) is a PEG that has a single, defined chain length and molecular weight. mdPEGs are typically generated by separation from the polymerization mixture by chromatography. In certain formulae, a monodisperse PEG moiety is assigned the abbreviation mdPEG.

In some aspects, the PEG is a Star PEG. Star PEGs have 10 to 100 PEG chains emanating from a central core group. In some aspects, the PEG is a Comb PEGs. Comb PEGs have multiple PEG chains normally grafted onto a polymer backbone.

In certain aspects, the PEG has a molar mass between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PEG is PEG₁₀₀, PEG₂₀₀, PEG₃₀₀, PEG₄₀₀, PEG₅₀₀, PEG₆₀₀, PEG₇₀₀, PEG₈₀₀, PEG₉₀₀, PEG₁₀₀₀, PEG₁₁₀₀, PEG₁₂₀₀, PEG₁₃₀₀, PEG₁₄₀₀, PEG₁₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₁₆₀₀, PEG₁₇₀₀, PEG₁₈₀₀, PEG₁₉₀₀, PEG₂₀₀₀, PEG₂₁₀₀, PEG₂₂₀₀, PEG₂₃₀₀, PEG₂₄₀₀, PEG₂₅₀₀, PEG₂₆₀₀, PEG₂₇₀₀, PEG₂₈₀₀, PEG₂₉₀₀, PEG₃₀₀₀, PEG₃₁₀₀, PEG₃₂₀₀, PEG₃₃₀₀, PEG₃₄₀₀, PEG₃₅₀₀, PEG₃₆₀₀, PEG₃₇₀₀, PEG₃₈₀₀, PEG₃₉₀₀, PEG₄₀₀₀, PEG₄₁₀₀, PEG₄₂₀₀, PEG₄₃₀₀, PEG₄₄₀₀, PEG₄₅₀₀, PEG₄₆₀₀, PEG₄₇₀₀, PEG₄₈₀₀, PEG₄₉₀₀, PEG₅₀₀₀, PEG₅₁₀₀, PEG₅₂₀₀, PEG₅₃₀₀, PEG₅₄₀₀, PEG₅₅₀₀, PEG₅₆₀₀, PEG₅₇₀₀, PEG₅₈₀₀, PEG₅₉₀₀, PEG₆₀₀₀, PEG₆₁₀₀, PEG₆₂₀₀, PEG₆₃₀₀, PEG₆₄₀₀, PEG₆₅₀₀, PEG₆₆₀₀, PEG₆₇₀₀, PEG₆₈₀₀, PEG₆₉₀₀, PEG₇₀₀₀, PEG₇₁₀₀, PEG₇₂₀₀, PEG₇₃₀₀, PEG₇₄₀₀, PEG₇₅₀₀, PEG₇₆₀₀, PEG₇₇₀₀, PEG₇₈₀₀, PEG₇₉₀₀, or PEG₈₀₀₀. In some aspects, the PEG is PEG₅₀₀₀. In some aspects, the PEG is PEG₆₀₀₀. In some aspects, the PEG is PEG₄₀₀₀.

In some aspects, the PEG is monodisperse, e.g., mPEG₁₀₀, mPEG₂₀₀, mPEG₃₀₀, mPEG₄₀₀, mPEG₅₀₀, mPEG₆₀₀, mPEG₇₀₀, mPEG₈₀₀, mPEG₉₀₀, mPEG₁₀₀₀, mPEG₁₁₀₀, mPEG₁₂₀₀, mPEG₁₃₀₀, mPEG₁₄₀₀, mPEG₁₅₀₀, mPEG₁₆₀₀, mPEG₁₇₀₀, mPEG₁₈₀₀, mPEG₁₉₀₀, mPEG₂₀₀₀, mPEG₂₁₀₀, mPEG₂₂₀₀, mPEG₂₃₀₀, mPEG₂₄₀₀, mPEG₂₅₀₀, mPEG₁₆₀₀, mPEG₁₇₀₀, mPEG₁₈₀₀, mPEG₁₉₀₀, mPEG₂₀₀₀, mPEG₂₁₀₀, mPEG₂₂₀₀, mPEG₂₃₀₀, mPEG₂₄₀₀, mPEG₂₅₀₀, mPEG₂₆₀₀, mPEG₂₇₀₀, mPEG₂₈₀₀, mPEG₂₉₀₀, mPEG₃₀₀₀, mPEG₃₁₀₀, mPEG₃₂₀₀, mPEG₃₃₀₀, mPEG₃₄₀₀, mPEG₃₅₀₀, mPEG₃₆₀₀, mPEG₃₇₀₀, mPEG₃₈₀₀, mPEG₃₉₀₀, mPEG₄₀₀₀, mPEG₄₁₀₀, mPEG₄₂₀₀, mPEG₄₃₀₀, mPEG₄₄₀₀, mPEG₄₅₀₀, mPEG₄₆₀₀, mPEG₄₇₀₀, mPEG₄₈₀₀, mPEG₄₉₀₀, mPEG₅₀₀₀, mPEG₅₁₀₀, mPEG₅₂₀₀, mPEG₅₃₀₀, mPEG₅₄₀₀, mPEG₅₅₀₀, mPEG₅₆₀₀, mPEG₅₇₀₀, mPEG₅₈₀₀, mPEG₅₉₀₀, mPEG₆₀₀₀, mPEG₆₁₀₀, mPEG₆₂₀₀, mPEG₆₃₀₀, mPEG₆₄₀₀, mPEG₆₅₀₀, mPEG₆₆₀₀, mPEG₆₇₀₀, mPEG₆₈₀₀, mPEG₆₉₀₀, mPEG₇₀₀₀, mPEG₇₁₀₀, mPEG₇₂₀₀, mPEG₇₃₀₀, mPEG₇₄₀₀, mPEG₇₅₀₀, mPEG₇₆₀₀, mPEG₇₇₀₀, mPEG₇₈₀₀, mPEG₇₉₀₀, or mPEG₈₀₀₀. In some aspects, the mPEG is mPEG₅₀₀₀. In some aspects, the mPEG is mPEG₆₀₀₀. In some aspects, the mPEG is mPEG₄₀₀₀.

In some aspects, the water-soluble biopolymer moiety is a polyglycerol (PG) described by the formula ((R₃—O—(CH₂—CHOH—CH₂O)ₙ—) with R₃ being hydrogen, methyl or ethyl, and n having a value from 3 to 200. In some aspects, the water-soluble biopolymer moiety is a branched polyglycerol described by the formula (R³—O—(CH₂—CHOR⁵—CH₂—O)ₙ—) with R⁵ being hydrogen or a linear glycerol chain described by the formula (R³—O—(CH₂—CHOH—CH₂—O)ₙ—) and R³ being hydrogen, methyl or ethyl. In some aspects, the water-soluble biopolymer moiety is a hyperbranched polyglycerol described by the formula (R³—O—(CH₂—CHOR⁵—CH₂—O)ₙ—) with R⁵ being hydrogen or a glycerol chain described by the formula (R³—O—(CH₂—CHOR⁶—CH₂—O)ₙ), with R⁶ being hydrogen or a glycerol chain described by the formula (R³—O—(CH₂—CHOR⁷—CH₂—O)ₙ—), with R⁷ being hydrogen or a linear glycerol chain described by the formula (R³—O—(CH₂—CHOH—CH₂—O)ₙ) and R³ being hydrogen, methyl or ethyl. Hyperbranched glycerol and methods for its synthesis are described in Oudshorn et al. (2006) Biomaterials 27:5471-5479; Wilms et al. (20100 Acc. Chem. Res. 43, 129-41, and references cited therein.

In certain aspects, the PG has a molar mass between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PG is PG₁₀₀, PG₂₀₀, PG₃₀₀, PG₄₀₀, PG₅₀₀, PG₆₀₀, PG₇₀₀, PG₈₀₀, PG₉₀₀, PG₁₀₀₀, PG₁₁₀₀, PG₁₂₀₀, PG₁₃₀₀, PG₁₄₀₀, PG₁₅₀₀, PG₁₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₁₇₀₀, PG₁₈₀₀, PG₁₉₀₀, PG₂₀₀₀, PG₂₁₀₀, PG₂₂₀₀, PG₂₃₀₀, PG₂₄₀₀, PG₂₅₀₀, PG₂₆₀₀, PG₂₇₀₀, PG₂₈₀₀, PG₂₉₀₀, PG₃₀₀₀, PG₃₁₀₀, PG₃₂₀₀, PG₃₃₀₀, PG₃₄₀₀, PG₃₅₀₀, PG₃₆₀₀, PG₃₇₀₀, PG₃₈₀₀, PG₃₉₀₀, PG₄₀₀₀, PG₄₁₀₀, PG₄₂₀₀, PG₄₃₀₀, PG₄₄₀₀, PG₄₅₀₀, PG₄₆₀₀, PG₄₇₀₀, PG₄₈₀₀, PG₄₉₀₀, PG₅₀₀₀, PG₅₁₀₀, PG₅₂₀₀, PG₅₃₀₀, PG₅₄₀₀, PG₅₅₀₀, PG₅₆₀₀, PG₅₇₀₀, PG₅₈₀₀, PG₅₉₀₀, PG₆₀₀₀, PG₆₁₀₀, PG₆₂₀₀, PG₆₃₀₀, PG₆₄₀₀, PG₆₅₀₀, PG₆₆₀₀, PG₆₇₀₀, PG₆₈₀₀, PG₆₉₀₀, PG₇₀₀₀, PG₇₁₀₀, PG₇₂₀₀, PG₇₃₀₀, PG₇₄₀₀, PG₇₅₀₀, PG₇₆₀₀, PG₇₇₀₀, PG₇₈₀₀, PG₇₉₀₀, or PG₈₀₀₀. In some aspects, the PG is PG₅₀₀₀. In some aspects, the PG is PG₆₀₀₀. In some aspects, the PG is PG₄₀₀₀.

In some aspects, the PG is monodisperse, e.g., mPG₁₀₀, mPG₂₀₀, mPG₃₀₀, mPG₄₀₀, mPG₅₀₀, mPF₆₀₀, mPG₇₀₀, mPG₈₀₀, mPG₉₀₀, mPG₁₀₀₀, mPG₁₁₀₀, mPG₁₂₀₀, mPG₁₃₀₀, mPG₁₄₀₀, mPG₁₅₀₀, mPG₁₆₀₀, mPG₁₇₀₀, mPG₁₈₀₀, mPG₁₉₀₀, mPG₂₀₀₀, mPG₂₁₀₀, mPG₂₂₀₀, mPG₂₃₀₀, mPG₂₄₀₀, mPG₂₅₀₀, mPG₁₆₀₀, mPG₁₇₀₀, mPG₁₈₀₀, mPG₁₉₀₀, mPG₂₀₀₀, mPG₂₁₀₀, mPG₂₂₀₀, mPG₂₃₀₀, mPG₂₄₀₀, mPG₂₅₀₀, mPG₂₆₀₀, mPG₂₇₀₀, mPG₂₈₀₀, mPG₂₉₀₀, mPG₃₀₀₀, mPG₃₁₀₀, mPG₃₂₀₀, mPG₃₃₀₀, mPG₃₄₀₀, mPG₃₅₀₀, mPG₃₆₀₀, mPG₃₇₀₀, mPG₃₈₀₀, mPG₃₉₀₀, mPG₄₀₀₀, mPG₄₁₀₀, mPG₄₂₀₀, mPG₄₃₀₀, mPG₄₄₀₀, mPG₄₅₀₀, mPG₄₆₀₀, mPG₄₇₀₀, mPG₄₈₀₀, mPG₄₉₀₀, mPG₅₀₀₀, mPG₅₁₀₀, mPG₅₂₀₀, mPG₅₃₀₀, mPG₅₄₀₀, mPG₅₅₀₀, mPG₅₆₀₀, mPG₅₇₀₀, mPG₅₈₀₀, mPG₅₉₀₀, mPG₆₀₀₀, mPG₆₁₀₀, mPG₆₂₀₀, mPG₆₃₀₀, mPG₆₄₀₀, mPG₆₅₀₀, mPG₆₆₀₀, mPG₆₇₀₀, mPG₆₈₀₀, mPG₆₉₀₀, mPG₇₀₀₀, mPG₇₁₀₀, mPG₇₂₀₀, mPG₇₃₀₀, mPG₇₄₀₀, mPG₇₅₀₀, mPG₇₆₀₀, mPG₇₇₀₀, mPG₇₈₀₀, mPG₇₉₀₀, or mPG₈₀₀₀.

In some aspects, the water-soluble biopolymer comprises poly(propylene glycol) (“PPG”). In some aspects, PPG is characterized by the following formula, with n having a value from 1 to 1000.

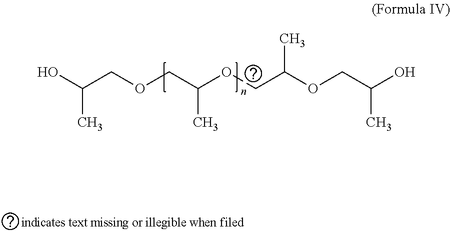

In some aspects, the n of the PPG has a value of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 189, 181, 182, 183, 184, 185, 186, 187, 188, 189, 190, 191, 192, 193, 194, 195, 196, 197, 198, 199, or 200.

In some aspects, n of the PPG is at least about 10, at least about 20, at least about 30, at least about 40, at least about 50, at least about 60, at least about 70, at least about 80, at least about 90, at least about 100, at least about 110, at least 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 440, at least about 450, at least about 460, at least about 470, at least about 480, at least about 490, at least about 500, at least about 510, at least about 520, at least about 530, at least about 540, at least about 550, at least about 560, at least about 670, at least about 580, at least about 590, at least about 600, at least about 610, at least about 620, at least about 630, at least about 640, at least about 650, at least about 660, at least about 670, at least about 680, at least about 690, at least about 700, at least about 710, at least about 720, at least about 730, at least about 740, at least about 750, at least about 760, at least about 770, at least about 780, at least about 790, at least about 800, at least about 810, at least about 820, at least about 830, at least about 840, at least about 850, at least about 860, at least about 870, at least about 880, at least about 890, at least about 900, at least about 910, at least about 920, at least about 930, at least about 940, at least about 950, at least about 960, at least about 970, at least about 980, at least about 990, or about 1000.

In some aspects, the n of the PPG is between about 50 and about 100, between about 100 and about 150, between about 150 and about 200, between about 200 and about 250, between about 250 and about 300, between about 300 and about 350, between about 350 and about 400, between about 400 and about 450, between about 450 and about 500, between about 500 and about 550, between about 550 and about 600, between about 600 and about 650, between about 650 and about 700, between about 700 and about 750, between about 750 and about 800, between about 800 and about 850, between about 850 and about 900, between about 900 and about 950, or between about 950 and about 1000.

In some aspects, the n of the PPG is at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, at least about 100, at least about 101, at least about 102, at least about 103, at least about 104, at least about 105, at least about 106, at least about 107, at least about 108, at least about 109, at least 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, at least about 141, at least about 142, at least about 143, at least about 144, at least about 145, at least about 146, at least about 147, at least about 148, at least about 149, at least about 150, at least about 151, at least about 152, at least about 153, at least about 154, at least about 155, at least about 156, at least about 157, at least about 158, at least about 159, or at least about 160.

In some aspects, the n of the PPG is about 80 to about 90, about 90 to about 100, about 100 to about 110, about 110 to about 120, about 120 to about 130, about 130 to about 140, about 140 to about 150, about 150 to about 160, about 85 to about 95, about 95 to about 105, about 105 to about 115, about 115 to about 125, about 125 to about 135, about 135 to about 145, about 145 to about 155, about 155 to about 165, about 80 to about 100, about 100 to about 120, about 120 to about 140, about 140 to about 160, about 85 to about 105, about 105 to about 125, about 125 to about 145, or about 145 to about 165.

Thus, is some aspects, the PPG is a branched PPG. Branched PPGs have three to ten PPG chains emanating from a central core group. In certain aspects, the PPG moiety is a monodisperse polyethylene glycol. In the context of the present disclosure, a monodisperse polyethylene glycol (mdPPG) is a PPG that has a single, defined chain length and molecular weight. mdPEGs are typically generated by separation from the polymerization mixture by chromatography. In certain formulae, a monodisperse PPG moiety is assigned the abbreviation mdPPG.

In some aspects, the PPG is a Star PPG. Star PPGs have 10 to 100 PPG chains emanating from a central core group. In some aspects, the PPG is a Comb PPGs. Comb PPGs have multiple PPG chains normally grafted onto a polymer backbone.

In certain aspects, the PPG has a molar mass between about 1000 g/mol and about 2000 g/mol, between about 2000 g/mol and about 3000 g/mol, between about 3000 g/mol to about 4000 g/mol, between about 4000 g/mol and about 5000 g/mol, between about 5000 g/mol and about 6000 g/mol, between about 6000 g/mol and about 7000 g/mol, or between 7000 g/mol and about 8000 g/mol.

In some aspects, the PPG is PPG₁₀₀, PPG₂₀₀, PPG₃₀₀, PPG₄₀₀, PPG₅₀₀, PPG₆₀₀, PPG₇₀₀, PPG₈₀₀, PPG₉₀₀, PPG₁₀₀₀, PPG₁₁₀₀, PPG₁₂₀₀, PPG₁₃₀₀, PPG₁₄₀₀, PPG₁₅₀₀, PPG₁₆₀₀, PPG₁₇₀₀, PPG₁₈₀₀, PPG₁₉₀₀, PPG₂₀₀₀, PPG₂₁₀₀, PPG₂₂₀₀, PPG₂₃₀₀, PPG₂₄₀₀, PPG₂₅₀₀, PPG₁₆₀₀, PPG₁₇₀₀, PPG₁₈₀₀, PPG₁₉₀₀, PPG₂₀₀₀, PPG₂₁₀₀, PPG₂₂₀₀, PPG₂₃₀₀, PPG₂₄₀₀, PPG₂₅₀₀, PPG₂₆₀₀, PPG₂₇₀₀, PPG₂₈₀₀, PPG₂₉₀₀, PPG₃₀₀₀, PPG₃₁₀₀, PPG₃₂₀₀, PPG₃₃₀₀, PPG₃₄₀₀, PPG₃₅₀₀, PPG₃₆₀₀, PPG₃₇₀₀, PPG₃₈₀₀, PPG₃₉₀₀, PPG₄₀₀₀, PPG₄₁₀₀, PPG₄₂₀₀, PPG₄₃₀₀, PPG₄₄₀₀, PPG₄₅₀₀, PPG₄₆₀₀, PPG₄₇₀₀, PPG₄₈₀₀, PPG₄₉₀₀, PPG₅₀₀₀, PPG₅₁₀₀, PPG₅₂₀₀, PPG₅₃₀₀, PPG₅₄₀₀, PPG₅₅₀₀, PPG₅₆₀₀, PPG₅₇₀₀, PPG₅₈₀₀, PPG₅₉₀₀, PPG₆₀₀₀, PPG₆₁₀₀, PPG₆₂₀₀, PPG₆₃₀₀, PPG₆₄₀₀, PPG₆₅₀₀, PPG₆₆₀₀, PPG₆₇₀₀, PPG₆₈₀₀, PPG₆₉₀₀, PPG₇₀₀₀, PPG₇₁₀₀, PPG₇₂₀₀, PPG₇₃₀₀, PPG₇₄₀₀, PPG₇₅₀₀, PPG₇₆₀₀, PPG₇₇₀₀, PPG₇₈₀₀, PPG₇₉₀₀, or PPG₈₀₀₀. In some aspects, the PPG is PPG₅₀₀₀. In some aspects, the PPG is PPG₆₀₀₀. In some aspects, the PPG is PPG₄₀₀₀.

In some aspects, the PPG is monodisperse, e.g., mPPG₁₀₀, mPPG₂₀₀, mPPG₃₀₀, mPPG₄₀₀, mPPG₅₀₀, mPPG₆₀₀, mPPG₇₀₀, mPPG₈₀₀, mPPG₉₀₀, mPPG₁₀₀₀, mPPG₁₁₀₀, mPPG₁₂₀₀, mPPG₁₃₀₀, mPPG₁₄₀₀, mPPG₁₅₀₀, mPPG₁₆₀₀, mPPG₁₇₀₀, mPPG₁₈₀₀, mPPG₁₉₀₀, mPPG₂₀₀₀, mPPG₂₁₀₀, mPPG₂₂₀₀, mPPG₂₃₀₀, mPPG₂₄₀₀, mPPG₂₅₀₀, mPPG₁₆₀₀, mPPG₁₇₀₀, mPPG₁₈₀₀, mPPG₁₉₀₀, mPPG₂₀₀₀, mPPG₂₁₀₀, mPPG₂₂₀₀, mPPG₂₃₀₀, mPPG₂₄₀₀, mPPG₂₅₀₀, mPPG₂₆₀₀, mPPG₂₇₀₀, mPPG₂₈₀₀, mPPG₂₉₀₀, mPPG₃₀₀₀, mPPG₃₁₀₀, mPPG₃₂₀₀, mPPG₃₃₀₀, mPPG₃₄₀₀, mPPG₃₅₀₀, mPPG₃₆₀₀, mPPG₃₇₀₀, mPPG₃₈₀₀, mPPG₃₉₀₀, mPPG₄₀₀₀, mPPG₄₁₀₀, mPPG₄₂₀₀, mPPG₄₃₀₀, mPPG₄₄₀₀, mPPG₄₅₀₀, mPPG₄₆₀₀, mPPG₄₇₀₀, mPPG₄₈₀₀, mPPG₄₉₀₀, mPPG₅₀₀₀, mPPG₅₁₀₀, mPPG₅₂₀₀, mPPG₅₃₀₀, mPPG₅₄₀₀, mPPG₅₅₀₀, mPPG₅₆₀₀, mPPG₅₇₀₀, mPPG₅₈₀₀, mPPG₅₉₀₀, mPPG₆₀₀₀, mPPG₆₁₀₀, mPPG₆₂₀₀, mPPG₆₃₀₀, mPPG₆₄₀₀, mPPG₆₅₀₀, mPPG₆₆₀₀, mPPG₆₇₀₀, mPPG₆₈₀₀, mPPG₆₉₀₀, mPPG₇₀₀₀, mPPG₇₁₀₀, mPPG₇₂₀₀, mPPG₇₃₀₀, mPPG₇₄₀₀, mPPG₇₅₀₀, mPPG₇₆₀₀, mPPG₇₇₀₀, m PPG₇₈₀₀, mPPG₇₉₀₀, or mPPG₈₀₀₀. In some aspects, the mPPG is mPPG₅₀₀₀. In some aspects, the mPPG is mPPG₆₀₀₀. In some aspects, the mPPG is mPPG₄₀₀₀.

### IV.A.2. Cationic Carrier

In some aspects, the cationic carrier units of the present disclosure comprise at least one cationic carrier moiety. The term “cationic carrier” refers to a moiety or portion of a cationic carrier unit of the present disclosure comprising a plurality of positive charges that can interact and bind electrostatically an anionic payload (or an anionic carrier attached to a payload). In some aspects, the number of positive charges or positively charged groups on the cationic carrier is similar to the number of negative charges or negatively charged groups on the anionic payload (or an anionic carrier attached to a payload). In some aspects, the cationic carrier comprises a biopolymer, e.g., a peptide (e.g., a polylysine).

In some aspects, the cationic carrier comprises one or more basic amino acids (e.g., lysine, arginine, histidine, or a combination thereof). In some aspects, the cationic carrier comprises at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, at least about 80, at least about 81, at least about 82, at least about 83, at least about 84, at least about 85, at least about 86, at least about 87, at least about 88, at least about 89, at least about 90, at least about 91, at least about 92, at least about 93, at least about 94, at least about 95, at least about 96, at least about 97, at least about 98, at least about 99, or at least about 100 basic amino acids, e.g., lysines, arginines, or combinations thereof. In some aspects, the cationic carrier moiety comprises about 60, about 70, about 80, about 90, or about 100 basic amino acids. In certain aspects, the cationic carrier moiety comprises about 80 basic amino acids.

In some aspects, the cationic carrier unit comprises at least about 40 basic amino acids, e.g., lysines. In some aspects, the cationic carrier unit comprises at least about 45 basic amino acids, e.g., lysines. In some aspects, the cationic carrier unit comprises at least about 50 basic amino acids, e.g., lysines. In some aspects, the cationic carrier unit comprises at least about 55 basic amino acids, e.g., lysines. In some aspects, the cationic carrier unit comprises at least about 60 basic amino acids, e.g., lysines. In some aspects, the cationic carrier unit comprises at least about 65 basic amino acids, e.g., lysines. In some aspects, the cationic carrier unit comprises at least about 70 basic amino acids, e.g., lysines. In some aspects, the cationic carrier unit comprises at least about 75 basic amino acids, e.g., lysines. In some aspects, the cationic carrier unit comprises at least about 80 basic amino acids, e.g., lysines.

In some aspects, the cationic carrier unit comprises about 30 to about 1000, about 30 to about 900, about 30 to about 800, about 30 to about 700, about 30 to about 600, about 30 to about 500, about 30 to about 400, about 30 to about 300, about 30 to about 200, about 30 to about 100, about 40 to about 1000, about 40 to about 900, about 40 to about 800, about 40 to about 700, about 40 to about 600, about 40 to about 500, about 40 to about 400, about 40 to about 300, about 40 to about 200, or about 40 to about 100 basic amino acids, e.g., lysines. In some aspects, the basic amino acids, e.g., lysines, are not modified such that they possess —NH3+(e.g., positive charge).

In some aspects, the cationic carrier unit comprises about 30 to about 100, about 30 to about 90, about 30 to about 80, about 30 to about 70, about 30 to about 60, about 30 to about 50, about 30 to about 40, about 40 to about 100, about 40 to about 90, about 40 to about 80, about 40 to about 70, about 40 to about 60, about 70 to about 80, about 75 to about 85, about 65 to about 75, about 65 to about 80, about 60 to about 85, or about 40 to about 500 basic amino acids, e.g., lysines.

In some aspects, the cationic carrier unit comprises about 100 to about 1000, about 100 to about 900, about 100 to about 800, about 100 to about 700, about 100 to about 600, about 100 to about 500, about 100 to about 400, about 100 to about 300, about 100 to about 200, about 200 to about 1000, about 200 to about 900, about 200 to about 800, about 200 to about 700, about 200 to about 600, about 200 to about 500, about 200 to about 400, about 200 to about 300, about 300 to about 1000, about 300 to about 900, about 300 to about 800, about 300 to about 700, about 300 to about 600, about 300 to about 500, about 300 to about 400, about 400 to about 1000, about 400 to about 900, about 400 to about 800, about 400 to about 700, about 400 to about 600, about 400 to about 500, about 500 to about 1000, about 500 to about 900, about 500 to about 800, about 500 to about 700, about 500 to about 600, about 600 to about 1000, about 600 to about 900, about 600 to about 800, about 600 to about 700, about 700 to about 1000, about 700 to about 900, about 700 to about 800, about 800 to about 1000, about 800 to about 900, or about 900 to about 1000 basic amino acids, e.g., lysines.

In some aspects, the number of basic amino acids, e.g., lysines, arginines, histidines, or combinations thereof, can be adjusted based on the length of the anionic payload. For example, an anionic payload with a longer sequence can be paired with higher number of basic amino acids, e.g., lysines. In some aspects, the number of basic amino acids, e.g., lysines, in the cationic carrier unit can be calculated so that the molar ratio of protonated amine in polymer to phosphate in an anionic payload, e.g., mRNA (N/P ratio) is at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, or at least about 20. In some aspects, the molar ratio of protonated amine in polymer to phosphate in an anionic payload, e.g., mRNA (N/P ratio) is between about 1 to about 20, about 1, about 2, about 3, about 4, about 5, about 6, about 7, about 8, about 9, about 10, about 11, about 12, about 13, about 14, about 15, about 16, about 17, about 18, about 19, or about 20. In some aspects, the number of basic amino acids, e.g., lysines, in the cationic carrier unit is calculated so that the molar ratio of protonated amine in polymer to phosphate in an anionic payload, e.g., mRNA (N/P ratio) is about 1 to about 10, e.g., about 3 to about 4, about 4 to about 5, about 5 to about 6, about 6 to about 7, or about 7 to about 8. In some aspects, the number of basic amino acids, e.g., lysines, in the cationic carrier unit is calculated so that the molar ratio of protonated amine in polymer to phosphate in an anionic payload, e.g., mRNA (N/P ratio) is about 1 to about 2. In some aspects, the number of basic amino acids, e.g., lysines, in the cationic carrier unit is calculated so that the molar ratio of protonated amine in polymer to phosphate in an anionic payload, e.g., mRNA (N/P ratio) is about 3 to about 4. In some aspects, the number of basic amino acids, e.g., lysines, in the cationic carrier unit is calculated so that the molar ratio of protonated amine in polymer to phosphate in an anionic payload, e.g., mRNA (N/P ratio) is about 2 to about 3. In some aspects, the number of basic amino acids, e.g., lysines, in the cationic carrier unit is calculated so that the molar ratio of protonated amine in polymer to phosphate in an anionic payload, e.g., mRNA (N/P ratio) is about 4 to about 5. In some aspects, the number of basic amino acids, e.g., lysines, in the cationic carrier unit is calculated so that the molar ratio of protonated amine in polymer to phosphate in an anionic payload, e.g., mRNA (N/P ratio) is about 5 to about 6. In some aspects, the number of basic amino acids, e.g., lysines, in the cationic carrier unit is calculated so that the molar ratio of protonated amine in polymer to phosphate in an anionic payload, e.g., mRNA (N/P ratio) is about 6 to about 7. In some aspects, the number of basic amino acids, e.g., lysines, in the cationic carrier unit is calculated so that the molar ratio of protonated amine in polymer to phosphate in an anionic payload, e.g., mRNA (N/P ratio) is about 7 to about 8. In some aspects, the number of basic amino acids, e.g., lysines, in the cationic carrier unit is calculated so that the molar ratio of protonated amine in polymer to phosphate in an anionic payload, e.g., mRNA (N/P ratio) is about 8 to about 9. In some aspects, the number of basic amino acids, e.g., lysines, in the cationic carrier unit is calculated so that the molar ratio of protonated amine in polymer to phosphate in an anionic payload, e.g., mRNA (N/P ratio) is about 9 to about 10.

A person of skill in the art would understand that since a role of the cationic carrier moiety is to neutralize negative charges on the payload (e.g., negative changes in the phosphate backbone of an mRNA) via electrostatic interaction, in some aspects (e.g., when the payload is a nucleic acid such as an antimir), the length of the cationic carrier, number of positively charged groups on the cationic carrier, and distribution and orientation of charges present on the cationic carrier will depend on the length and charge distribution on the payload molecule.

In some aspects, the cationic carrier comprises between about 5 and about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about and about 70, between about 70 and about 75, or between about 75 and about 80 basic amino acids. In some specific aspects, the positively charged carrier comprises between 30 and about 50 basic amino acids. In some specific aspects, the positively charged carrier comprises between 70 and about 80 basic amino acids.

In some aspects, the basic amino acid comprises arginine, lysine, histidine, or any combination thereof. In some aspects, the basic amino acid is a D-amino acid. In some aspects, the basic amino acid is an L-amino acid. In some aspects, the positively charged carrier comprises D-amino acids and L-amino acids. In some aspects, the basic amino acid comprises at least one unnatural amino acid or a derivative thereof. In some aspects, the basic amino acid is arginine, lysine, histidine, L-4-aminomethyl-phenylalanine, L-4-guanidine-phenylalanine, L-4-aminomethyl-N-isopropyl-phenylalanine, L-3-pyridyl-alanine, L-trans-4-aminomethylcyclohexyl-alanine, L-4-piperidinyl-alanine, L-4-aminocyclohexyl-alanine, 4-guanidinobutyric acid, L-2-amino-3-guanidinopropionic acid, DL-5-hydroxylysine, pyrrolysine, 5-hydroxy-L-lysine, methyllysine, hypusine, or any combination thereof. In a particular aspect, the positively charged carrier comprises about 40 lysines. In a particular aspect, the positively charged carrier comprises about 50 lysines. In a particular aspect, the positively charged carrier comprises about 60 lysines. In a particular aspect, the positively charged carrier comprises about 70 lysines. In a particular aspect, the positively charged carrier comprises about 80 lysines.

In other aspects, the cationic carrier comprises a polymer or copolymer comprising at least three, at least four, at least five, at least six, at least seven, at least eight, at least nine, at least ten, at least 11, at least 12, at least 13, at least 14, at last 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, or at least 80 cationic groups (e.g., amino groups). In some aspects, the cationic carrier comprises a polymer or copolymer comprising between about 5 and about 10 cationic groups, between about 10 and about 15 cationic groups, between about 15 and about 20 cationic groups, between about 20 and about 25 cationic groups, between about 25 and about 30 cationic groups, between about 30 and about 35 cationic groups, between about 35 and about 40 cationic groups, between about 40 and about 45 cationic groups, between about 45 and about 50 cationic groups, between about 50 and about 55 cationic groups, between about 55 and about 60 cationic groups, between about 60 and about 65 cationic groups, between about 65 and about 70 cationic groups, between about 70 and about 75 cationic groups, or between about 45 and about 50 cationic groups (e.g., amino groups). In some specific aspects, the cationic carrier comprises a polymer or copolymer comprising between 30 and about 50 cationic groups (e.g., amino groups). In some specific aspects, the cationic carrier comprises a polymer or copolymer comprising between 70 and about 80 cationic groups (e.g., amino groups). In some aspects, the polymer or copolymer is an acrylate, a polyalcohol, or a polysaccharide.

In some aspects, the cationic carrier moiety binds to a single payload molecule. In other aspects, a cationic carrier moiety can bind to multiple payload molecules, which may be identical or different.

In some aspects, the positive charges of the cationic carrier moiety and negative charges of a nucleic acid payload are at an ionic ratio of about 20:1, about 19:1, about 18:1, about 17:1, about 16:1, about 15:1, about 14:1, about 13:1, about 12:1, about 11:1, about 10:1, about 9:1, about 8:1 about 7:1, about 6:1 about 5:1, about 4:1, about 3:1, about 2:1, or about 1:1. In some aspects, the negative charges of a nucleic acid payload and the positive charges of the cationic carrier moiety are at an ionic ratio of about 20:1, about 19:1, about 18:1, about 17:1, about 16:1, about 15:1, about 14:1, about 13:1, about 12:1, about 11:1, about 10:1, about 9:1, about 8:1 about 7:1, about 6:1 about 5:1, about 4:1, about 3:1, about 2:1, or about 1:1.

In some aspects, the anionic payload comprises a nucleotide sequence having about 10 to about 1000 (e.g., about 100 to about 1000) in length, wherein the N/P ratio of the the cationic carrier moiety and the anionic payload is about 2 to about 10, e.g., about 2 to about 9, about 2 to about 8, about 2 to about 7, about 2 to about 6, about 2 to about 5, about 2 to about 4, about 2 to about 3, e.g., e.g., about 2, about 3, about 4, about 5, about 6, about 7, about 8, about 9, or about 10. In some aspects, an N/P ratio of the cationic carrier moiety and the anionic payload of about 10 to about 1000 nucleotides in length is between about 1 and about 10, e.g., about 1, about 2, about 3, about 4, about 5, about 6, about 7, about 8, about 9, or about 10.

In some aspects, the anionic payload comprises a nucleotide sequence having about 1000 to about 2000 in length, wherein the N/P ratio of the cationic carrier moiety and the anionic payload is about 3 to about 12, e.g., about 3, about 4, about 5, about 6, about 7, about 8, about 9, about 10, about 11, or about 12. In some aspects, the N/P ratio of the cationic carrier moiety and the anionic payload is between about 4 and about 7, e.g., about 4, about 5, about 6, or about 7.

In some aspects, the anionic payload comprises a nucleotide sequence having about 2000 to about 3000 in length, wherein the N/P ratio of the cationic carrier moiety and the anionic payload is about 3 to about 16, e.g., about 3, about 4, about 5, about 6, about 7, about 8, about 9, about 10, about 11, about 12, about 13, about 14, about 15, about 16. In some aspects, wherein the N/P ratio of the cationic carrier moiety and the anionic payload is between about 6 and about 9, e.g., about 6, about 7, about 8, or about 9.

In some aspects, the anionic payload comprises a nucleotide sequence having about 3000 to about 4000 in length, wherein the N/P ratio of the cationic carrier moiety and the anionic payload is about 3 to about 20, e.g., about 3, about 4, about 5, about 6, about 7, about 8, about 9, about 10, about 11, about 12, about 13, about 14, about 15, about 16, about 17, about 18, about 19, or about 20. In some aspects, wherein the N/P ratio of the cationic carrier moiety and the anionic payload is between about 7 and about 10, e.g., about 7, about 8, about 9, or about 10.

In some aspects, the cationic carrier moiety has a free terminus wherein the end group is a reactive group. In some aspects, the cationic carrier moiety has a free terminus (e.g., the C-terminus in a poly-lysine cationic carrier moiety) wherein the end group is an amino (—NH₂) group. In some aspects, the cationic carrier moiety has a free terminus wherein the end group is an sulfhydryl group. In some aspects, the reactive group of the cationic carrier moiety is attached to a hydrophobic moiety, e.g., a vitamin B3 hydrophobic moiety.

### IV.A.3. Crosslinking Moiety

In some aspects, the cationic carrier units of the present disclosure comprise at least one crosslinking moiety. The term “crosslinking moiety” refers to a moiety or portion of a polymer block comprising a plurality of agents that are capable of forming crosslinks. In some aspects, the number of agents that are capable of forming crosslinks comprises an amino acid with a side chain of a crosslinking agent. In some aspects, the CM comprises a biopolymer, e.g., a peptide (e.g., a polylysine) linked to a crosslinking agent.

In some aspects, the crosslinking moiety comprises one or more amino acids (e.g., lysine, arginine, histidine, or a combination thereof). In some aspects, the crosslinking moiety comprises at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 amino acids, e.g., lysines, arginines, or combinations thereof, each of which is linked to a crosslinking agent.

In some aspects, the crosslinking moiety comprises at least about 10 amino acids, e.g., lysines, each of which is linked to a crosslinking agent. In some aspects, the crosslinking moiety comprises at least about 11 amino acids, e.g., lysines, each of which is linked to a crosslinking agent. In some aspects, the crosslinking moiety comprises at least about 12 amino acids, e.g., lysines, each of which is linked to a crosslinking agent. In some aspects, the crosslinking moiety comprises at least about 13 amino acids, e.g., lysines, each of which is linked to a crosslinking agent. In some aspects, the crosslinking moiety comprises at least about 14 amino acids, e.g., lysines, each of which is linked to a crosslinking agent. In some aspects, the crosslinking moiety comprises at least about 15 amino acids, e.g., lysines, each of which is linked to a crosslinking agent. In some aspects, the crosslinking moiety comprises at least about 16 amino acids, e.g., lysines, each of which is linked to a crosslinking agent. In some aspects, the crosslinking moiety comprises at least about 17 amino acids, e.g., lysines, each of which is linked to a crosslinking agent. In some aspects, the crosslinking moiety comprises at least about 18 amino acids, e.g., lysines, each of which is linked to a crosslinking agent. In some aspects, the crosslinking moiety comprises at least about 19 amino acids, e.g., lysines, each of which is linked to a crosslinking agent. In some aspects, the crosslinking moiety comprises at least about 20 amino acids, e.g., lysines, each of which is linked to a crosslinking agent.

In some aspects, a crosslinking agent is a thiol. In some aspects, a crosslinking agent is a thiol derivative.

### IV.A.4. Hydrophobic Moiety

In some aspects, the cationic carrier units of the present disclosure comprise at least one hydrophobic moiety. The term “hydrophobic moiety”, as used herein, refers to a molecular entity that can, e.g., (i) complement the therapeutic or prophylactic activity of the payload, (ii) modulate the therapeutic or prophylactic activity of the payload, (iii) function as a therapeutic and/or prophylactic agent in the target tissue or target cells, (iv) facilitate the transport of the cationic carrier unit across a physiological barrier, e.g., the BBB and/or the plasma membrane, (v) improve the homeostasis of the target tissue or target cell, (vi) contribute positively charges groups to the cationic carried moiety, or (vii) any combination thereof.

In some aspects, the hydrophobic moiety is capable of modulating, e.g., an immune response, an inflammatory response, or a tissue microenvironment.

In some aspects, a hydrophobic moiety capable of modulating an immune response can comprise, e.g., tyrosine or dopamine. Tyrosine can be transformed into L-DOPA, and then be converted to dopamine via 2-step enzymatic reaction. Normally, dopamine levels are low in the Parkinson's disease patients. Therefore, in some aspects, tyrosine is a hydrophobic moiety in cationic carrier units used for the treatment of Parkinson's disease. Tryptophan can be converted to serotonin, a neurotransmitter thought to play a role in appetite, emotions, and motor, cognitive, and autonomic functions. Accordingly, in some aspects, cationic carrier units of the present disclosure used for the treatment of disease or conditions related to low serotonin levels comprise tryptophan as a hydrophobic moiety.

In some aspects, a hydrophobic moiety can modulate a tumor microenvironment in a subject with a tumor, for example, by inhibiting or reducing hypoxia in the tumor microenvironment.

In some aspects, the hydrophobic moiety comprises, e.g., an amino acid linked to an imidazole derivative, a vitamin, or any combination thereof.

In some aspects, the hydrophobic moiety comprises an amino acid (e.g., lysine) linked to an imidazole derivative comprising:

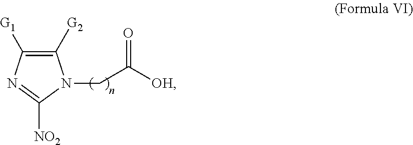

wherein each of G₁ and G₂ is independently H, an aromatic ring, or 1-10 alkyl, or G₁ and G₂ together form an aromatic ring, and wherein n is 1-10.

In some aspects, the hydrophobic moiety comprises an amino acid (e.g., lysine) linked to nitroimidazole. Nitroimidazoles function as antibiotics. Nitroheterocycles in nitroimidazoles can be reductively activated in hypoxic cells, and then undergo redox recycling or decompose to cytotoxic products. Reduction usually happens only in anaerobic bacteria or in anoxic tissues, therefore, they have relative little effect upon human cells or aerobic bacteria. In some aspects, the hydrophobic moiety comprises an amino acid (e.g., lysine) linked to metronidazole, tinidazole, nimorazole, dimetridazole, pretomanid, ornidazole, megazol, azanidazole, benznidazole, nitroimidazole, or any combination thereof.

In some aspects, the hydrophobic moiety comprises

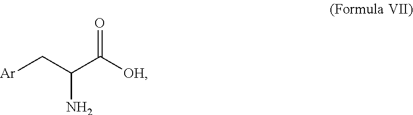

wherein Ar is

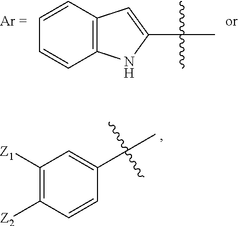

and
wherein each of Z1 and Z2 is H or OH.

In some aspects, the hydrophobic moiety is capable of inhibiting or reducing an inflammatory response.

In some aspects, the hydrophobic moiety is an amino acid (e.g., lysine) linked to a vitamin. In some aspects, the vitamin comprises a cyclic ring or cyclic hetero atom ring and a carboxyl group or hydroxyl group. In some aspects, the vitamin comprises:

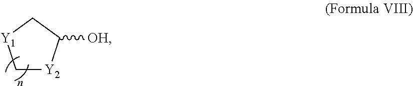

wherein each of Y1 and Y2 is C, N, O, or S, and wherein n is 1 or 2.

In some aspects, the vitamin is selected from the group consisting of vitamin A (retinol), vitamin B1 (Thiamine Chloride), vitamin B2 (Riboflavin), vitamin B3 (Niacinamide), vitamin B6 (Pyridoxal), vitamin B7 (Biotin), vitamin B9 (Folic acid), vitamin B12 (Cobalamin), vitamin C (Ascorbic acid), vitamin D2, vitamin D3, vitamin E (Tocopherol), vitamin M, vitamin H, a derivative thereof, and any combination thereof.

In some aspects, the vitamin is vitamin B3 (also known as niacin or nicotinic acid).

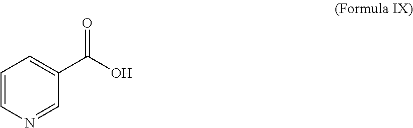

In some aspects, the hydrophobic moiety comprises at least about one, at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 amino acids (e.g., lysines), each of which is linked to vitamin B3. In some aspects, the hydrophobic moiety comprises about 1 amino acid (e.g., lysine), each of which is linked to vitamin B3. In some aspects, the hydrophobic moiety comprises about 2 amino acids (e.g., lysines), each of which is linked to vitamin B3. In some aspects, the hydrophobic moiety comprises about 3 amino acids (e.g., lysines), each of which is linked to vitamin B3. In some aspects, the hydrophobic moiety comprises about 4 amino acids (e.g., lysines), each of which is linked to vitamin B3. In some aspects, the hydrophobic moiety comprises about 5 amino acids (e.g., lysines), each of which is linked to vitamin B3. In some aspects, the hydrophobic moiety comprises about 6 amino acids (e.g., lysines), each of which is linked to vitamin B3. In some aspects, the hydrophobic moiety comprises about 7 amino acids (e.g., lysines), each of which is linked to vitamin B3. In some aspects, the hydrophobic moiety comprises about 8 amino acids (e.g., lysines), each of which is linked to vitamin B3. In some aspects, the hydrophobic moiety comprises about 9 amino acids (e.g., lysines), each of which is linked to vitamin B3. In some aspects, the hydrophobic moiety comprises about 10 amino acids (e.g., lysines), each of which is linked to vitamin B3. In some aspects, the hydrophobic moiety comprises about 11 amino acids (e.g., lysines), each of which is linked to vitamin B3. In some aspects, the hydrophobic moiety comprises about 12 amino acids (e.g., lysines), each of which is linked to vitamin B3. In some aspects, the hydrophobic moiety comprises about 13 amino acids (e.g., lysines), each of which is linked to vitamin B3. In some aspects, the hydrophobic moiety comprises about 14 amino acids (e.g., lysines), each of which is linked to vitamin B3. In some aspects, the hydrophobic moiety comprises about 15 amino acids (e.g., lysines), each of which is linked to vitamin B3. In some aspects, the hydrophobic moiety comprises about 16 amino acids (e.g., lysines), each of which is linked to vitamin B3. In some aspects, the hydrophobic moiety comprises about 17 amino acids (e.g., lysines), each of which is linked to vitamin B3. In some aspects, the hydrophobic moiety comprises about 18 amino acids (e.g., lysines), each of which is linked to vitamin B3. In some aspects, the hydrophobic moiety comprises about 19 amino acids (e.g., lysines), each of which is linked to vitamin B3. In some aspects, the hydrophobic moiety comprises about 20 amino acids (e.g., lysines), each of which is linked to vitamin B3.

In some aspects, the hydrophobic moiety comprises from about 1 to about 10 amino acids (e.g., lysines), each of which is linked to vitamin B3, about 5 to about 10 amino acids (e.g., lysines), each of which is linked to vitamin B3, about 10 to about 15 amino acids (e.g., lysines), each of which is linked to vitamin B3, about 15 to about 20 amino acids (e.g., lysines), each of which is linked to vitamin B3, about 1 to about 20 vitamin amino acids (e.g., lysines), each of which is linked to B3, about 1 to about 15 vitamin amino acids (e.g., lysines), each of which is linked to B3, about 1 to about 10 amino acids (e.g., lysines), each of which is linked to vitamin B3, about 1 to about 5 amino acids (e.g., lysines), each of which is linked to vitamin B3.

Niacin is a precursor of the coenzymes nicotinamide adenine dinucleotide (NAD) and nicotinamide adenine dinucleotide phosphate (NADP) in vivo. NAD converts to NADP by phosphorylation in the presence of the enzyme NAD+ kinase. NADP and NAD are coenzymes for many dehydrogenases, participating in many hydrogen transfer processes. NAD is important in catabolism of fat, carbohydrate, protein, and alcohol, as well as cell signaling and DNA repair, and NADP mostly in anabolism reactions such as fatty acid and cholesterol synthesis. High energy requirements (brain) or high turnover rate (gut, skin) organs are usually the most susceptible to their deficiency.

Niacin produces marked anti-inflammatory effects in a variety of tissues—including the brain, gastrointestinal tract, skin, and vascular tissue—through the activation of NIACR1. Niacin has been shown to attenuate neuroinflammation and may have efficacy in treating neuroimmune disorders such as multiple sclerosis and Parkinson's disease. See Offermanns & Schwaninger (2015) Trends in Molecular Medicine 21:245-266; Chai et al (2013) Current Atherosclerosis Reports 15:325; Graff et al. (2016) Metabolism 65:102-13; and Wakade & Chong (2014) Journal of the Neurological Sciences 347:34-8, which are herein incorporated by reference in their entireties.

### IV.A.5. Targeting Moiety

In some aspects, the cationic carrier unit comprises a targeting moiety, which is linked to the water-soluble polymer optionally via a linker. As used herein, the term “targeting moiety” refers to a biorecognition molecule that binds to a specific biological substance or site. In some aspects, the targeting moiety is specific for a certain target molecule (e.g., a ligand targeting a receptor, or an antibody targeting a surface protein), tissue (e.g., a molecule that would preferentially carry the micelle to a specific organ or tissue, e.g., liver, brain, or endothelium), or facilitate transport through a physiological barrier (e.g., a peptide or other molecule that may facilitate transport across the brain blood barrier or plasma membrane).

For targeting a payload (e.g., a nucleotide molecule, e.g., an mRNA) according to the present disclosure, a targeting moiety can be coupled to a cationic carrier unit, and therefore, to the external surface of a micelle, whereas the micelle has the payload entrapped within its core.

In some aspects, the targeting moiety is a targeting moiety capable of targeting the micelle of the present disclosure to a tissue. In some aspects, the tissue is liver, brain, kidney, lung, ovary, pancreas, thyroid, breast, stomach, or any combination thereof. In some aspects, the tissue is cancer tissue, e.g., liver cancer, brain cancer, kidney cancer, lung cancer, ovary cancer, pancreas cancer, thyroid cancer, breast cancer, stomach cancer, or any combination thereof.

In a specific aspect, the tissue is liver. In a specific aspect, the targeting moiety targeting liver is cholesterol. In other aspects, the targeting moiety targeting liver is a ligand that binds an asialoglycoprotein receptor targeting moiety. In some aspects, the asialoglycoprotein receptor targeting moiety comprises a GalNAc cluster. In some aspects, the GalNAc cluster is a monovalent, divalent, trivalent, or tetravalent GalNAc cluster.

In another aspect, the tissue is pancreas. In some aspects, the targeting moiety targeting pancreas comprises a ligand targeting αvβ3 integrin receptors on pancreatic cells. In some aspects, the targeting moiety comprises an arginylglycylaspartic acid (RGD) peptide sequence (L-Arginyl-Glycyl-L-Aspartic acid; Arg-Gly-Asp).

In some aspects, the tissue is a tissue in the central nervous system, e.g., neural tissue. In some aspects, the targeting moiety targeting the central nervous system is capable being transported by Large-neutral Amino Acid Transporter 1 (LAT1). LAT1 (SLC7A5) is a transporter for both the uptake of large neutral amino acids and a number of pharmaceutical drugs. LAT1 can transport drugs such as L-dopa or gabapentin.

In some aspects, a targeting moiety comprises glucose, e.g., D-glucose, which can bind to Glucose transporter 1 (or GLUT1) and cross BBB. GLUT1, also known as solute carrier family 2, facilitated glucose transporter member 1 (SLC2A1), is a uniporter protein that in humans is encoded by the SLC2A1 gene. GLUT1 facilitates the transport of glucose across the plasma membranes of mammalian cells. This gene encodes a major glucose transporter in the mammalian blood-brain barrier.

In some aspects, a targeting moiety comprises galactose, e.g., D-galactose, which can bind to GLUT1 transporter to cross BBB. In some aspects, a targeting moiety comprises glutamic acid, which can bind to acetylcholinesterase inhibitor (AChEI) and/or EAATs inhibitors and cross BBB. Acetylcholinesterase is the enzyme that is the primary member of the cholinesterase enzyme family. An acetylcholinesterase inhibitor (AChEI) is the inhibitor that inhibits acetylcholinesterase from breaking down acetylcholine into choline and acetate, thereby increasing both the level and duration of action of the neurotransmitter acetylcholine in the central nervous system, autonomic ganglia and neuromuscular junctions, which are rich in acetylcholine receptors. Acetylcholinesterase inhibitors are one of two types of cholinesterase inhibitors; the other being butyryl-cholinesterase inhibitors.

In some aspects, the tissue targeted by a targeting moiety is a skeletal muscle. In some aspects, the targeting moiety targeting skeletal muscle is capable being transported by Large-neutral Amino Acid Transporter 1 (LAT1).

It is expressed in numerous cell types including T-cells, cancer cells and brain endothelial cells. LAT1 is consistently expressed at high levels in brain microvessel endothelial cells. Being a solute carrier located primarily in the BBB, targeting the micelles of the present disclosure to LAT1 allows delivery through the BBB. In some aspects, the targeting moiety targeting a micelle of the present disclosure to the LAT1 transporter is an amino acid, e.g., a branched-chain or aromatic amino acid. In some aspects, the amino acid is valine, leucine, and/or isoleucine. In some aspects, the amino acid is tryptophan and/or tyrosine. In some aspects, the amino acid is tryptophan. In other aspects, the amino acid is tyrosine.

In some aspects, the targeting moiety is a LAT1 ligand selected from tryptophan, tyrosine, phenylalanine, tryptophan, methionine, thyroxine, melphalan, L-DOPA, gabapentin, 3,5-I-diiodotyrosine, 3-iodo-I-tyrosine, fenclonine, acivicin, leucine, BCH, methionine, histidine, valine, or any combination thereof.

See Singh & Ecker (2018) “Insights into the Structure, Function, and Ligand Discovery of the Large Neutral Amino Acid Transporter 1, LAT1,” Int. J. Mol. Sci. 19:1278; Geier et al. (2013) “Structure-based ligand discovery for the Large-neutral Amino Acid Transporter 1, LAT-1,” Proc. Natl. Acad. Sci. USA 110:5480-85; and Chien et al. (2018) “Reevaluating the Substrate Specificity of the L-type Amino Acid Transporter (LAT1),” J. Med. Chem. 61:7358-73, which are herein incorporated by reference in their entireties.

Non-limiting examples of targeting moieties are described below.

IV.A.5.a. Ligands

A ligand functions as a type of targeting moiety defined as a selectively bindable material that has a selective (or specific), affinity for another substance. The ligand is recognized and bound by a usually, but not necessarily, larger specific binding body or “binding partner,” or “receptor.” Examples of ligands suitable for targeting are antigens, haptens, biotin, biotin derivatives, lectins, galactosamine and fucosylamine moieties, receptors, substrates, coenzymes and cofactors among others.

When applied to the micelles of the present disclosure a ligand includes an antigen or hapten that is capable of being bound by, or to, its corresponding antibody or fraction thereof. Also included are viral antigens or hemagglutinins and neuraminidases and nucleocapsids including those from any DNA and RNA viruses, AIDS, HIV and hepatitis viruses, adenoviruses, alphaviruses, arenaviruses, coronaviruses, flaviviruses, herpesviruses, myxoviruses, oncornaviruses, papovaviruses, paramyxoviruses, parvoviruses, picornaviruses, poxviruses, reoviruses, rhabdoviruses, rhinoviruses, togaviruses and viroids; any bacterial antigens including those of gram-negative and gram-positive bacteria, Acinetobacter, Achromobacter, Bacteroides, Clostridium, Chlamydia, enterobacteria, Haemophilus, Lactobacillus, Neisseria, Staphyloccus, or Streptoccocus; any fungal antigens including those of Aspergillus, Candida, Coccidiodes, mycoses, phycomycetes, and yeasts; any mycoplasma antigens; any rickettsial antigens; any protozoan antigens; any parasite antigens; any human antigens including those of blood cells, virus infected cells, genetic markers, heart diseases, oncoproteins, plasma proteins, complement factors, rheumatoid factors. Included are cancer and tumor antigens such as alpha-fetoproteins, prostate specific antigen (PSA) and CEA, cancer markers and oncoproteins, among others.

Other substances that can function as ligands for targeting a micelle of the present disclosure are certain vitamins (i.e. folic acid, B₁₂), steroids, prostaglandins, carbohydrates, lipids, antibiotics, drugs, digoxins, pesticides, narcotics, neuro-transmitters, and substances used or modified such that they function as ligands.

In some aspects, the targeting moiety comprises a protein or protein fragment (e.g., hormones, toxins), and synthetic or natural polypeptides with cell affinity. Ligands also include various substances with selective affinity for ligators that are produced through recombinant DNA, genetic and molecular engineering. Except when stated otherwise, ligands of the instant disclosure also include ligands as defined in U.S. Pat. No. 3,817,837, which is herein incorporated by reference in its entirety.

IV.A.5.b. Ligators

A ligator functions as a type of targeting moiety defined for this disclosure as a specific binding body or “partner” or “receptor,” that is usually, but not necessarily, larger than the ligand it can bind to. For the purposes of this disclosure, it can be a specific substance or material or chemical or “reactant” that is capable of selective affinity binding with a specific ligand. A ligator can be a protein such as an antibody, a nonprotein binding body, or a “specific reactor.”

When applied to this disclosure, a ligator includes an antibody, which is defined to include all classes of antibodies, monoclonal antibodies, chimeric antibodies, Fab fractions, fragments and derivatives thereof. The term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, scFab, (scFab)₂, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the targeting moiety is an antibody or a molecule comprising an antigen binding fragment thereof. In some aspects, the antibody is a nanobody. In some aspects, the antibody is an ADC. The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the targeting moiety is an antibody-drug conjugate.

Under certain conditions, the instant disclosure is also applicable to using other substances as ligators. For instance, other ligators suitable for targeting include naturally occurring receptors, any hemagglutinins and cell membrane and nuclear derivatives that bind specifically to hormones, vitamins, drugs, antibiotics, cancer markers, genetic markers, viruses, and histocompatibility markers. Another group of ligators includes any RNA and DNA binding substances such as polyethylenimine (PEI) and polypeptides or proteins such as histones and protamines.

Other ligators also include enzymes, especially cell surface enzymes such as neuraminidases, plasma proteins, avidins, streptavidins, chalones, cavitands, thyroglobulin, intrinsic factor, globulins, chelators, surfactants, organometallic substances, staphylococcal protein A, protein G, ribosomes, bacteriophages, cytochromes, lectins, certain resins, and organic polymers.

Targeting moieties also include various substances such as any proteins, protein fragments or polypeptides with affinity for the surface of any cells, tissues or microorganisms that are produced through recombinant DNA, genetic and molecular engineering. Thus, in some aspects, the targeting moiety directs a micelle of the present disclosure to a specific tissue (i.e., liver tissue or brain tissue), to a specific type of cell (e.g., a certain type of cancer cells), or to a physiological compartment or physiological barrier (e.g., the BBB).

### IV.A.6. Linkers

As described above, a cationic carrier unit disclosed herein can comprise one or more linkers. As used herein, the term “linker” refers to a peptide or polypeptide sequence (e.g., a synthetic peptide or polypeptide sequence), or a non-peptide linker for which its main function is to connect two moieties in a cationic carrier unit disclosed herein. In some aspects, cationic carrier units of the present disclosure can comprise at least one linker connecting a tissue-specific targeting moiety (TM) with a water soluble polymer (WS), at least one linker connecting a water-soluble biopolymer (WP) with cationic carrier (CC) or a hydrophobic moiety (HM) or a crosslinking moiety (CM), at least one linker connecting a cationic carrier (CC) with a hydrophobic moiety (HM), or any combination thereof. In some aspects, two or more linkers can be linked in tandem.

When multiple linkers are present in a cationic carrier unit disclosed herein, each of the linkers can be the same or different. Generally, linkers provide flexibility to the cationic carrier unit. Linkers are not typically cleaved; however, in certain aspects, such cleavage can be desirable. Accordingly, in some aspects a linker can comprise one or more protease-cleavable sites, which can be located within the sequence of the linker or flanking the linker at either end of the linker sequence.

In one aspect, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker can comprise at least about 110, at least about 120, at least about 130, at least about 140, at least about 150, at least about 160, at least about 170, at least about 180, at least about 190, or at least about 200 amino acids.

In other aspects, the peptide linker can comprise at least about 200, at least about 250, at least about 300, at least about 350, at least about 400, at least about 450, at least about 500, at least 550, at least about 600, at least about 650, at least about 700, at least about 750, at least about 800, at least about 850, at least about 900, at least about 950, or at least about 1,000 amino acids.

The peptide linker can comprise between 1 and about 5 amino acids, between 1 and about 10 amino acids, between 1 and about 20 amino acids, between about 10 and about 50 amino acids, between about 50 and about 100 amino acids, between about 100 and about 200 amino acids, between about 200 and about 300 amino acids, between about 300 and about 400 amino acids, between about 400 and about 500 amino acids, between about 500 and about 600 amino acids, between about 600 and about 700 amino acids, between about 700 and about 800 amino acids, between about 800 and about 900 amino acids, or between about 900 and about 1000 amino acids.

Examples of peptide linkers are well known in the art. In some aspects, the linker is a glycine/serine linker. In some aspects, the peptide linker is glycine/serine linker according to the formula [(Gly)n-Ser]m where n is any integer from 1 to 100 and m is any integer from 1 to 100. In other aspects the glycine/serine linker is according to the formula [(Gly)x-Sery]z (SEQ ID NO: 1) wherein x in an integer from 1 to 4, y is 0 or 1, and z is an integers from 1 to 50. In one aspect, the peptide linker comprises the sequence Gn, where n can be an integer from 1 to 100. In a specific aspect, the sequence of the peptide linker is GGGG (SEQ ID NO: 2).

In some aspects, the peptide linker can comprise the sequence (GlyAla)n (SEQ ID NO: 3), wherein n is an integer between 1 and 100. In other aspects, the peptide linker can comprise the sequence (GlyGlySer)n (SEQ ID NO: 4), wherein n is an integer between 1 and 100.

In other aspects, the peptide linker comprises the sequence (GGGS)n (SEQ ID NO: 5). In still other aspects, the peptide linker comprises the sequence (GGS)n(GGGGS)n (SEQ ID NO: 6). In these instances, n can be an integer from 1-100. In other instances, n can be an integer from one to 20, i.e., 1, 2, 3, 4, 5, 6, 7, 8, 9, 10, 11, 12, 13, 14, 15, 16, 17, 18, 19, or 20.

Examples of linkers include, but are not limited to, GGG, SGGSGGS (SEQ ID NO: 7), GGSGGSGGSGGSGGG (SEQ ID NO: 8), GGSGGSGGGGSGGGGS (SEQ ID NO: 9), GGSGGSGGSGGSGGSGGS (SEQ ID NO: 10), or GGGGSGGGGSGGGGS (SEQ ID NO: 11). In other aspects, the linker is a poly-G sequence (GGGG)n (SEQ ID NO: 12), where n can be an integer from 1-100.

In one aspect, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion). In another aspect, the peptide linker can comprise non-naturally occurring amino acids. In another aspect, the peptide linker can comprise naturally occurring amino acids occurring in a linear sequence that does not occur in nature. In still another aspect, the peptide linker can comprise a naturally occurring polypeptide sequence.

In some aspects, the linker comprises a non-peptide linker. In other aspects, the linker consists of a non-peptide linker. In some aspects, the non-peptide linker can be, e.g., maleimido caproyl (MC), maleimido propanoyl (MP), methoxyl polyethyleneglycol (MPEG), succinimidyl 4-(N-maleimidomethyl)-cyclohexane-1-carboxylate (SMCC), m-maleimidobenzoyl-N-hydroxysuccinimide ester (MBS), succinimidyl 4-(p-maleimidophenyl)butyrate (SMPB), N-succinimidyl(4-iodoacetyl)aminobenzonate (SIAB), succinimidyl 6-[3-(2-pyridyldithio)-propionamide]hexanoate (LC-SPDP), 4-succinimidyloxycarbonyl-alpha-methyl-alpha-(2-pyridyldithio)toluene (SMPT), etc. (see, e.g., U.S. Pat. No. 7,375,078).

Linkers can be introduced into polypeptide sequences using techniques known in the art (e.g., chemical conjugation, recombinant techniques, or peptide synthesis). Modifications can be confirmed by DNA sequence analysis. In some aspects, the linkers can be introduced using recombinant techniques. In other aspects, the linkers can be introduced using solid phase peptide synthesis. In certain aspects, a cationic carrier unit disclosed herein can contain simultaneously one or more linkers that have been introduced using recombinant techniques and one or more linkers that have been introduced using solid phase peptide synthesis or methods of chemical conjugation known in the art. In some aspects, the linker comprises a cleavage site.

### V. Cells

In some aspects, provided herein are cells that have been modified to comprise a polynucleotide described herein (e.g., comprising an ORF, HA-5′-UTR, and a HA-3′-UTR). In certain aspects, the cells comprise a vector (e.g., AAV or lentivirus vector) that comprises a polynucleotide described herein.

Not to be bound by any one theory, in some aspects, the cells described herein (i.e., comprising a polynucleotide of the present disclosure or a vector comprising the polynucleotide) are useful for producing a protein (e.g., therapeutic protein described herein, e.g., coronavirus protein). As described herein, in some aspects, polynucleotides of the present disclosure (e.g., comprising an ORF, HA-5′-UTR, and a HA-3′-UTR) are capable of increasing the expression of the encoded protein in a cell. Accordingly, in some aspects, a cell described herein (i.e., comprising a polynucleotide of the present disclosure or a vector comprising the polynucleotide) produces greater expression of the encoded protein compared to a reference cell. In certain aspects, the reference cell comprises a polynucleotide lacking the HA-5′-UTR and/or HA-3′-UTR described herein). In some aspects, expression of an encoded protein (e.g., therapeutic protein described herein) in a cell described herein is increased by at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 11-fold, at least about 12-fold, at least about 13-fold, at least about 14-fold, at least about 15-fold, at least about 16-fold, at least about 17-fold, at least about 18-fold, at least about 19-fold, at least about 20-fold, at least about 25-fold, at least about 30-fold, at least about 35-fold, at least about 40-fold, at least about 45-fold, at least about 50-fold, at least about 75-fold, or at least about 100-fold or more, compared to the corresponding expression in the reference cell.

In some aspects, the cells described herein (i.e., comprising a polynucleotide of the present disclosure or a vector comprising the polynucleotide) can produce the encoded protein in vitro. In certain aspects, the cells described herein (i.e., comprising a polynucleotide of the present disclosure or a vector comprising the polynucleotide) can produce the encoded protein in vivo. For instance, in some aspects, a polynucleotide described herein or a vector comprising the polynucleotide can be introduced into a cell ex vivo (e.g., via transfection), and then the cell can be administered to a subject (e.g., adoptive cell therapy), wherein the encoded protein is produced in the subject after the administration. In some aspects, a polynucleotide described herein or a vector comprising the polynucleotide can be administered to a subject, e.g., as part of a gene therapy. In some aspects, the cells described herein (i.e., comprising a polynucleotide described herein or a vector comprising the polynucleotide) can produce the encoded protein both in vitro and in vivo.

Any suitable cells known in the art can be modified to comprise a polynucleotide described herein (e.g., comprising an ORF, a HA-5′-UTR, and a HA-3′-UTR) or a vector comprising the polynucleotide. In some aspects, a cell that can be used to produce (e.g., in vivo) a protein encoded by a polynucleotide of the present disclosure comprises a human cell. In certain aspects, the human cell is a cell of a subject that is to receive an administration of a polynucleotide described herein or a vector comprising the polynucleotide. In certain aspects, the human cell is from a donor (e.g., healthy human subject).

In some aspects, a cell that can be used to produce (e.g., in vitro) a protein encoded by a polynucleotide of the present disclosure comprises a host cell. In some aspects, the host cell is a eukaryotic cell. In some aspects, the host cell is selected from the group consisting of a mammalian cell, an insect cell, a yeast cell, a transgenic mammalian cell, a plant cell, and any combination thereof. In some aspects, the host cell is a prokaryotic cell. In some aspects, the prokaryotic cell is a bacterial cell.

In some aspects, the host cell is a mammalian cell. Non-limiting examples of mammalian host cells that are suitable for the present disclosure include: CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7O3O, COS (e.g., COS1 or COS), PER.C6, VERO, HsS78Bst, HEK-293T, HepG2, SP210, R1.1, B-W, L-M, BSC1, BSC40, YB/20, BMT10, HBK, NSO, HT1080, HsS78Bst cells, and combinations thereof.

### VI. Pharmaceutical Compositions

As is apparent from the present disclosure, any of the polynucleotides, vectors, proteins (e.g., therapeutic protein encoded by a polynucleotide described herein), and cells described herein (also referred to herein as “active compounds”) can be incorporated into pharmaceutical compositions suitable for administration. Accordingly, in some aspects, a pharmaceutical composition comprises the active compound and a pharmaceutically acceptable excipient.

As used herein, the term “pharmaceutically acceptable excipient” (also referred to herein as “pharmaceutically acceptable carrier”) comprises any and all solvents, dispersion media, coatings, antibacterial and antifungal agents, isotonic and absorption delaying agents, and the like, compatible with pharmaceutical administration. The use of such media and agents for pharmaceutically active compounds is well known in the art. Except insofar as any conventional media or agent is incompatible with the active compound, use thereof in the compositions is contemplated. Supplementary active compounds can also be incorporated into the compositions.

In some aspects, disclosed herein is a pharmaceutical composition comprising (a) a polynucleotide described herein (e.g., comprising an ORF, a HA-5′-UTR, and a HA-3′-UTR) and (b) a pharmaceutically acceptable excipient. In some aspects, disclosed herein is a pharmaceutical composition comprising (a) a vector (e.g., AAV or lentivirus vector) as described herein and (b) a pharmaceutically acceptable excipient. In some aspects, disclosed herein is a pharmaceutical composition comprising (a) a cell as described herein (e.g., modified to comprise a polynucleotide of the present disclosure) and (b) a pharmaceutically acceptable excipient.

A pharmaceutical composition of the present disclosure is formulated to be compatible with its intended route of administration. In some aspects, a suitable route of administration that can be used with the present disclosure comprises intramuscular administration. In some aspects, a suitable route of administration includes intranasal administration. Additional non-limiting examples of suitable routes of administration include parenteral, e.g., intravenous, intradermal, subcutaneous, oral, transdermal (topical), and transmucosal, and any combination thereof. Another route of administration includes pulmonary administration. In addition, it can be desirable to administer a therapeutically effective amount of the pharmaceutical composition locally to an area in need of treatment. This can be achieved by, for example, local or regional infusion or perfusion during surgery, topical application, injection, catheter, suppository, or implant (for example, implants formed from porous, non-porous, or gelatinous materials, including membranes, such as sialastic membranes or fibers), and the like. In some aspects, the therapeutically effective amount of the pharmaceutical composition is delivered in a vesicle, such as liposomes (see, e.g., Langer, Science 249:1527-33, 1990 and Treat et al., in Liposomes in the Therapy of Infectious Disease and Cancer, Lopez Berestein and Fidler (eds.), Liss, N.Y., pp. 353-65, 1989).

In some aspects, a pharmaceutical composition described herein can be delivered in a controlled release system. For instance, in certain aspects, a pump can be used (see, e.g., Langer, Science 249:1527-33, 1990; Sefton, Crit. Rev. Biomed. Eng. 14:201-40, 1987; Buchwald et al., Surgery 88:507-16, 1980; Saudek et al., N Engl. J Med. 321:574-79, 1989). In some aspects, polymeric materials can be used (see, e.g., Levy et al., Science 228:190-92, 1985; During et al., Ann. Neural. 25:351-56, 1989; Howard et al., J Neurosurg. 71:105-12, 1989). Other controlled release systems, such as those discussed by Langer (Science 249:1527-33, 1990), can also be used.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN© 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor ELS (BASF; Parsippany, NJ), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation can be vacuum drying and freeze-drying, which yields a powder of the active ingredient plus any additional desired ingredient from a previously sterile-filtered solution thereof.

For administration by inhalation, the compounds are delivered in the form of an aerosol spray from a pressurized container or dispenser that contains a suitable propellant, e.g., a gas such as carbon dioxide, or a nebulizer. Systemic administration can also be by transmucosal or transdermal means.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In some aspects, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

In some aspects, active compounds of the present disclosure can be formulated in dosage unit form for ease of administration and uniformity of dosage. Dosage unit form as used herein refers to physically discrete units suited as unitary dosages for the subject to be treated with each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such a functional compound for the treatment of individuals. The pharmaceutical compositions can be included in a container, pack, or dispenser together with instructions for administration.

### VII. Kits

The present disclosure also provides kits or products of manufacture, comprising any of the polynucleotides, vectors, encoded proteins, cells, and/or pharmaceutical compositions described herein, and optionally instructions for use, e.g., instructions for use according to the methods disclosed herein. Accordingly, in some aspects, disclosed herein is a kit comprising (i) a polynucleotide described herein (e.g., comprising an ORF, a HA-5′-UTR, and a HA-3′-UTR), and (ii) instructions for use. In some aspects, disclosed herein is a kit comprising (i) a vector described herein, and (ii) instructions for use. In some aspects, disclosed herein is a kit comprising (i) a cell described herein, and (ii) instructions for use. In some aspects, disclosed herein is a kit comprising (i) a pharmaceutical composition described herein, and (ii) instructions for use.

In some aspects, the kit or product of manufacture comprises a polynucleotide, vector, encoded protein, cell, and/or pharmaceutical composition described herein in a single container. In certain aspects, the kit or product of manufacture comprises the a polynucleotide, vector, encoded protein, cell, and/or pharmaceutical composition described herein in multiple (e.g., at least two) containers. one or more containers. One skilled in the art will readily recognize that any of the vectors, polynucleotides, cells, proteins, and pharmaceutical compositions of the present disclosure can be readily incorporated into one of the established kit formats which are well known in the art.

### VIII. Uses and Methods

### VIII.A. Methods of Producing

Also disclosed herein are methods of producing a polynucleotide described herein (e.g., comprising an ORF, a HA-5′-UTR, and a HA-3′-UTR). In some aspects, the polynucleotides described herein can be obtained, and the nucleotide sequence of the polynucleotides determined, by any method known in the art. Nucleotide sequences encoding protein of interest (e.g., therapeutic protein, e.g., coronavirus protein) can be determined using methods well known in the art, i.e., nucleotide codons known to encode particular amino acids are assembled in such a way to generate a nucleic acid that encodes the polypeptides. Such a polynucleotide encoding the polypeptide can be assembled from chemically synthesized oligonucleotides (e.g., as described in Kutmeier G et al., (1994), BioTechniques 17: 242-6), which, briefly, involves the synthesis of overlapping oligonucleotides containing portions of the sequence encoding the polypeptide, annealing and ligating of those oligonucleotides, and then amplification of the ligated oligonucleotides by PCR.

If a clone containing a nucleic acid encoding a particular polypeptide is not available, but the sequence of the polypeptide molecule is known, a nucleic acid encoding the polypeptide can be chemically synthesized or obtained from a suitable source (e.g., a cDNA library or a cDNA library generated from, or nucleic acid, preferably poly A+ RNA, isolated from, any tissue or cells expressing the proteins of interest, such as cells selected to express a polypeptide described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptides. Amplified nucleic acids generated by PCR can then be cloned into replicable cloning vectors using any method well known in the art.

Additional description of exemplary methods that can be used to produce the polynucleotides described herein are provided, e.g., in U.S. Pat. No. 9,597,380; US 2013/0259923; and US 2013/0115272; each of which is incorporated herein by reference in its entirety.

DNA encoding polypeptides described herein can be readily isolated and sequenced using conventional procedures (e.g., by using oligonucleotide probes that are capable of binding specifically to genes encoding the polypeptides disclosed herein). Many cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of polypeptides in the recombinant host cells.

Also provided herein are methods of producing a protein of interest (e.g., therapeutic protein, e.g., coronavirus protein). In some aspects, such a method comprises culturing a cell described herein under suitable conditions and, optionally, recovering the protein of interest. In certain aspects, a method of producing a protein of interest comprises administering a polynucleotide of the present disclosure to a subject in need thereof, such that the encoded protein is produced in the subject. Additional disclosure relating to such in vivo method of producing a protein is provided elsewhere in the present disclosure (see, e.g., “therapeutic uses”).

### VII.B. Therapeutic Uses

As is apparent from the present disclosure, compositions provided herein (e.g., polynucleotides, vectors, proteins, cells, and pharmaceutical compositions) have numerous in vitro and in vivo utilities. For example, a polynucleotide described herein (e.g., comprising an ORF, a HA-5′-UTR, and a HA-3′-UTR) can be administered to cells in culture, in vitro or ex vivo, or to human subjects, e.g., in vivo, to treat diseases.

Accordingly, in some aspects, the present disclosure is directed to therapeutic methods using any of the polynucleotides, vectors, proteins, cells, and pharmaceutical compositions described herein. In some aspects, disclosed herein is a method of expressing a protein of interest (e.g., therapeutic protein) in a subject in need thereof, comprising administering to the subject a polynucleotide of the present disclosure (e.g., comprising an ORF encoding the protein of interest, a HA-5′-UTR, and a HA-3′-UTR), wherein the protein of interest is expressed in the subject after the administration.

As described herein, the UTRs of the present disclosure (e.g., HA-5′-UTR and HA-3′-UTR) can increase the expression of a protein encoded by a polynucleotide when translated. Accordingly, in certain aspects, provided herein is a method of increasing the expression of a protein, comprising contacting a cell with a polynucleotide described herein (e.g., comprising an ORF encoding the protein, a HA-5′-UTR, and a HA-3′-UTR) or a vector comprising the polynucleotide. In some aspects, the contacting occurs in vivo (e.g., gene therapy). In certain aspects, the contacting occurs ex vivo. In some aspects, contacting the cell with a polynucleotide described herein results in increased protein expression compared to contacting the cell with a corresponding polynucleotide that lacks the HA-5′-UTR and HA-3′-UTR. In certain aspects, the protein expression is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least 25-fold, at least about 30-fold, at least about 35-fold, at least about 40-fold, at least about 45-fold, at least about 50-fold, at least about 75-fold, or at least about 100-fold or more.

In some aspects, disclosed herein is a method of treating a disease or disorder in a subject in need thereof comprising administering to the subject any of the polynucleotides, vectors, proteins, cells, and/or pharmaceutical compositions described herein. As is apparent from the present disclosure, in certain aspects, the administering results in the production of the encoded protein in the subject. Not to be bound by any one theory, in some aspects, the encoded protein when produced can regulate (e.g., induce or suppress) an immune response (e.g., cellular immune response and/or antibody-mediated immune response) in the subject, and thereby, treat the disease or disorder. In certain aspects, the encoded protein when produced can regulate other biological processes within the subject. Non-limiting examples of proteins (e.g., therapeutic proteins) that can be encoded by a polynucleotide described herein are provided elsewhere in the present disclosure (see Section II.B. “Open Reading Frame”).

Accordingly, in certain aspects, the present disclosure provides a method of inducing an immune response in a subject in need thereof, comprising administering to the subject a polynucleotide of the present disclosure (e.g., comprising an ORF encoding a heterologous protein, a HA-5′-UTR, and a HA-3′-UTR), wherein after the administration an immune response against the heterologous protein is induced in the subject. In some aspects, the present disclosure provides a method of suppressing an immune response in a subject in need thereof, comprising administering to the subject a polynucleotide of the present disclosure, wherein the encoded protein is capable of suppressing an immune response (e.g., by inducing the activation and/or proliferation of suppressive cells, e.g., regulatory T cells) in a subject.

Based on the disclosures provided herein, it will be apparent to those skilled in the art that any disease or disorder can be treated with the present disclosure, with the caveat that the encoded protein is capable of exerting a therapeutic effect on the disease or disorder. In certain aspects, a disease or disorder that can be treated with the present disclosure comprises a viral infection (or a disease or disorder associated with the viral infection). In some aspects, a viral infection comprises a coronavirus infection, influenza virus infection, or both.

As is apparent from the present disclosure, in some aspects, a disease or disorder that can be treated with the present disclosure comprises a cancer. Non-limiting examples of cancers include a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or combinations thereof.

In some aspects, a disease or disorder that can be treated with the present disclosure comprises a genetic disorder. In certain aspects, the genetic disorder comprises a Hunter syndrome.

In some aspects, the polynucleotides, vectors, proteins, cells, and pharmaceutical compositions described herein can be administered intravenously, transdermally, intradermally, subcutaneously, orally, pulmonarily, or any combination thereof. In some aspects, polynucleotides, vectors, proteins, cells, and pharmaceutical compositions described herein can be administered via a topical, epidermal mucosal, intranasal, oral, vaginal, rectal, sublingual, topical, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, or intrasternal route. In some aspects, any of the compositions of the present disclosure (e.g., polynucleotides, vectors, proteins, cells, and pharmaceutical compositions) are administered to a subject intramuscularly. In some aspects, the compositions of the present disclosure (e.g., polynucleotides, vectors, proteins, cells, and pharmaceutical compositions) are administered to a subject intravenously. In some aspects, the compositions of the present disclosure (e.g., polynucleotides, vectors, proteins, cells, and pharmaceutical compositions) are administered to a subject subcutaneously.

The following examples are offered by way of illustration and not by way of limitation.

### EXAMPLES

### Example 1: Construction of Modified RNA Construct

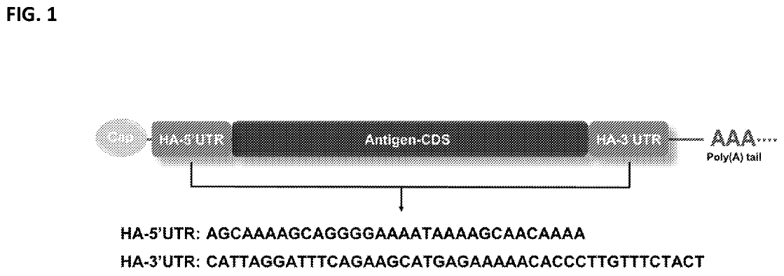

To construct a modified polynucleotide described herein, the 5′-UTR (SEQ ID NO: 13) and 3′-UTR (SEQ ID NO: 14) sequences of the hemagglutinin (HA) protein of the 2009 pandemic influenza virus (H1N1pdm09_A/Korea/01/09) were used. Briefly, for mRNA template production, the coding sequence of plasmids were amplified with DNA polymerase (KOD-Plus-Neo, TOYOBO). Transcriptions were performed at 37° C. for 2 hours using T7 polymerase (EZ™ MEGA T7 transcription kit, Enzynomics) and purified by LiCl precipitation. RNA was then capped using the vaccinia capping system (NEB) and mRNA Cap 2′-O-methyltransferase (NEB) and purified by LiCl precipitation. The 3′-poly(A) tail was added using E. coli poly(A) polymerase (NEB) and purified by LiCl precipitation. FIG. 1 provides an exemplary RNA construct described herein.

### Example 2: In Vitro Analysis of Influenza HA Protein Expression

To assess the ability of the modified polynucleotides to induce protein expression, the 5′-UTR and 3′-UTR sequences described in Example 1 were applied to a mRNA encoding the influenza HA (2009 pH1N1) protein (“modified mRNA construct” or “mod-mRNA”). Then, the HA protein expression was assessed using both an immunofluorescence assay and western blot.

For the immunofluorescence assay, HEK293T cells were seeded on coverslip in a 24-well plate (7×10⁴ cells/well) at 37° C. in Dulbecco's modified Eagle's medium (DMEM; Welgene Inc.). The cells were then transfected with either the modified mRNA construct (0.5 μg) or a control mRNA construct (0.5 μg) (i.e., corresponding mRNA that lack the 5′-UTR and 3′-UTR sequences described above) using Lipofectamine2000 (Invitrogen Life Technologies Inc.). Non-transfected cells were used as control. After 24 hours, the cells were fixed with 4% formaldehyde for 20 minutes and then, blocked with 2.5% BSA in PBS solution for 1 hour at room temperature. Afterwards, the cells were incubated for 2 hours at room temperature with the primary anti-HA antibody (Invitrogen Life Technologies Inc.), followed by a 1 hour incubation at room temperature with the secondary anti-rabbit Alexa 488 conjugated antibody. After the staining with the antibodies, the coverslips were mounted on slide glass with mounting medium containing 4,6-diamidino-2-phenylindole (DAPI) and imaged using confocal microscopy (Leica Biosystems).

For the western blot, HEK293T cells were seeded in a 6-well plate (3.5×10⁵ cells/well) at 37° C. in Dulbecco's modified Eagle's medium (DMEM; Welgene Inc.). The cells were then transfected with either the modified mRNA construct (3 μg) or the control mRNA construct (3 μg) using Lipofectamine2000 (Invitrogen Life Technologies Inc.). Non-transfected cells were used as control. After 6 hours or 24 hours after transfection, the cells were harvested and total protein was extracted with RIPA buffer (Sigma-Aldrich) and quantified using a protein BCA assay kit (Thermo Scientific). Then, the proteins (50 μg) were loaded onto a 10% SDS-PAGE gel membrane, and the resolved proteins were transferred to PVDF (Milipore) and blocked overnight with 3% (w/v) skim milk in PBS supplemented with 0.1% (v/v) Tween-20 at 4° C. Afterwards, the membrane was stained with the primary anti-HA antibody (Invitrogen Life Technologies Inc.) for 2 hours at room temperature. β-actin was used as the normalizing control (Cell signaling tech.). Next, the membrane was washed 3-times with PBS/T and incubated with anti-mouse or rabbit-conjugated HRP for 1 hour at room temperature. After the incubation, the membrane was incubated with ECL solution (GE healthcare) for visualization using LAS500 system (GE healthcare).

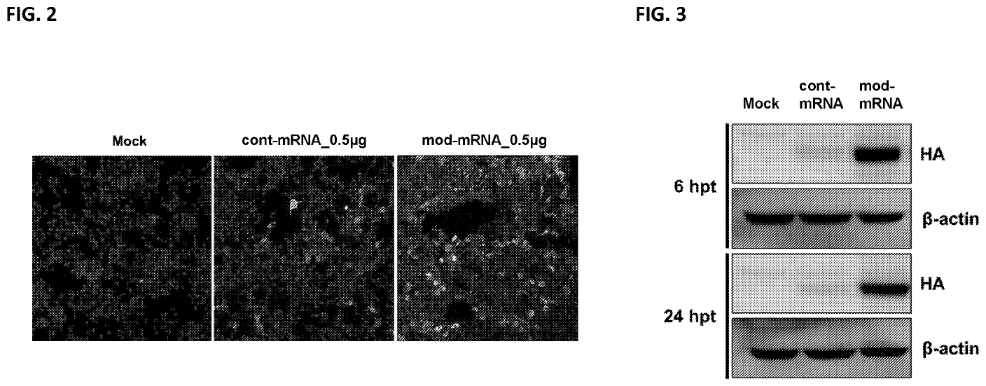

As shown in FIG. 2, HEK293T cells transfected with the modified mRNA construct (i.e., comprising the 5′-UTR and 3′-UTR sequences described herein) exhibited the greatest amount of HA protein expression as measured by immunofluorescence assay. Similar results were observed using western blot (see FIG. 3). At both 6-hours and 24-hours post-transfection, cells transfected with the modified mRNA construct expressed much greater amount of HA protein compared to cells transfected with the control mRNA construct, which lacked the 5′-UTR and 3′-UTR sequences.

The above results demonstrate that the inclusion of the 5′-UTR and 3′-UTR sequences described herein can increase the translation efficiency of a mRNA construct encoding a HA protein.

### Example 3: In Vitro Analysis of EGFP Protein Expression

To assess the ability of the 5′-UTR and 3′-UTR sequences described herein to increase the expression of non-influenza proteins, modified mRNA constructs encoding EGFP were constructed using the methods described in Example 1.

Briefly, HEK293T cells were seeded in a 6-well plate (3.5×10⁵ cells/well) at 37° C. in Dulbecco's modified Eagle's medium (DMEM; Welgene Inc.). The cells were then transfected with either the modified mRNA construct (i.e., comprising the 5′-UTR and 3′-UTR sequences described in Example 1) (3 μg) or the control mRNA construct (3 μg) (i.e., corresponding construct that does not comprise the 5′-UTR and 3′-UTR sequences described in Example 1) using Lipofectamine2000 (Invitrogen Life Technologies Inc.). At hours 6, 12, and 24 post-transfection, EGFP signal was measured using a fluorescent microscope (Leica Biosystems).

For western blot analysis, the HEK293 T cells were transfected with the modified mRNA construct or the control mRNA construct at two different doses using Lipofectamine2000 (Invitrogen Life Technologies Inc.): 1 μg and 3 μg. At 24-hours post transfection, total protein was extracted with RIPA buffer (Sigma-Aldrich) and quantified using a protein BCA assay kit (Thermo Scientific). Then, 10% SDS-PAGE gel membrane was used to separate out the proteins and analyzed (see Example 2).

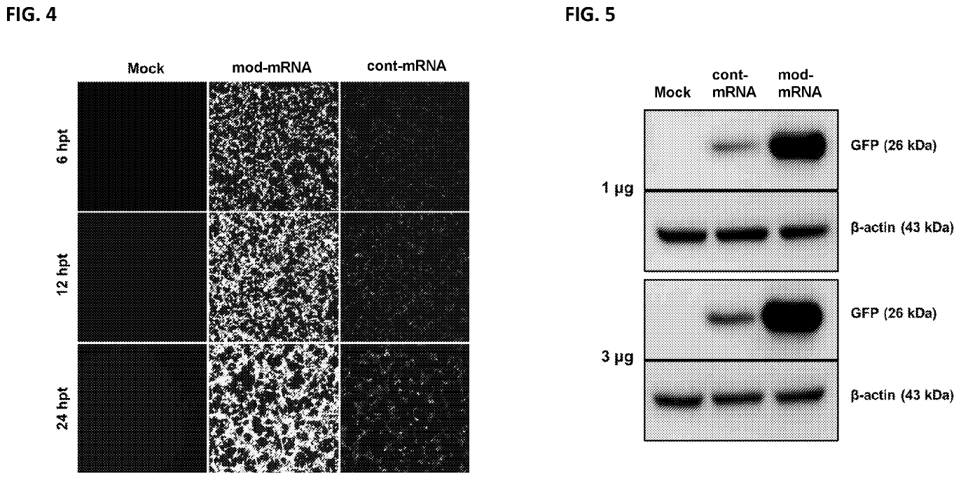

Similar to the results observed for the HA protein, cells transfected with the modified mRNA construct encoding EGFP exhibited much greater EGFP expression compared to both the mock control cells and cells transfected with the non-modified control mRNA constructs, as measured by fluorescence microscopy and western blot (see FIGS. 4 and 5, respectively).

Next, to assess whether the 5′-UTR and 3′-UTR sequences described herein can increase the expression of the proteins at the individual cell level, HEK293T cells were seeded in a 6-well plate (3.5×10⁵ cells/well) at 37° C. in Dulbecco's modified Eagle's medium (DMEM; Welgene Inc.) supplemented with 2% fetal bovine serum (Welgene Inc.). Then, the cells were transfected with either the modified mRNA encoding EGFP (i.e., comprising the 5′-UTR and 3′-UTR sequences) or the control modified mRNA, which also encodes EGFP but does not comprise the 5′-UTR and 3′-UTR sequences described in Example 1. Non-transfected cells were used as control (“Mock”). At 24 hours post-transfection, EGFP-positive cells were analyzed suing flow cytometer (BD Biosciences). In particular, 20,000 cells were collected from each of the groups, and the EGFP signal was captured through a fluorescein isothiocyanate (FITC) channel.

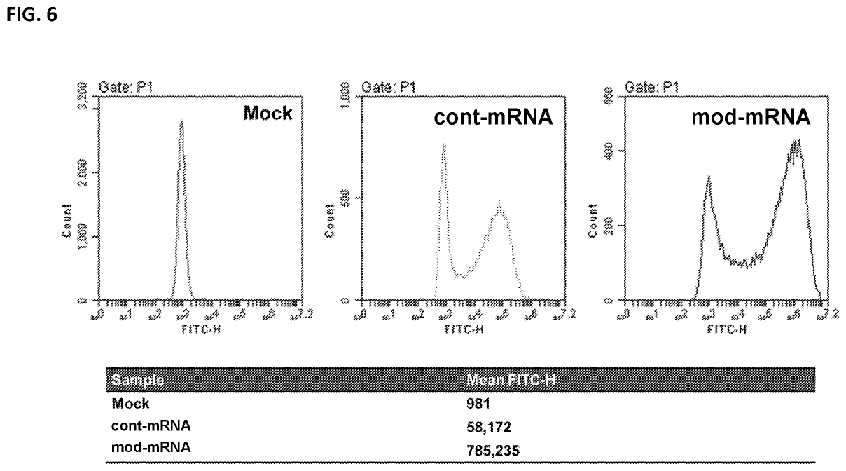

As shown in FIG. 6, among the EGFP+ cells from the different groups, the average EGFP expression level was much greater when the cells were transfected with the modified mRNA construct as compared to the control mRNA construct.

Collectively, the above results confirm the ability of the 5′-UTR and 3′-UTR sequences provided herein to increase cellular expression of EGFP.

### Example 4: In Vitro Analysis of Firefly Luciferase (Luc) Protein Expression

To confirm the results provided above, modified mRNA constructs encoding firefly luciferase (Luc) were constructed as described in Example 1. Then, HEK293T cells were seeded in a 6-well plate as described in Examples 2 and 3. The cells were then transfected with the modified mRNA construct encoding Luc (i.e., comprising the 5′-UTR and 3′-UTR sequences described in Example 1) or the control mRNA construct which lacks the 5′-UTR and 3′-UTR sequences. The mRNA constructs were transfected at two different doses: 1 μg and 3 μg. At 24 hours post-transfection, cells were treated with D-luciferin (150 ag), incubated for 5 minutes in the dark, and luciferase signal was subsequently visualized using IVIS imaging system (PerkinElmer). Using the methods provided in Example 2, LUC protein expression was also assessed using western blot.

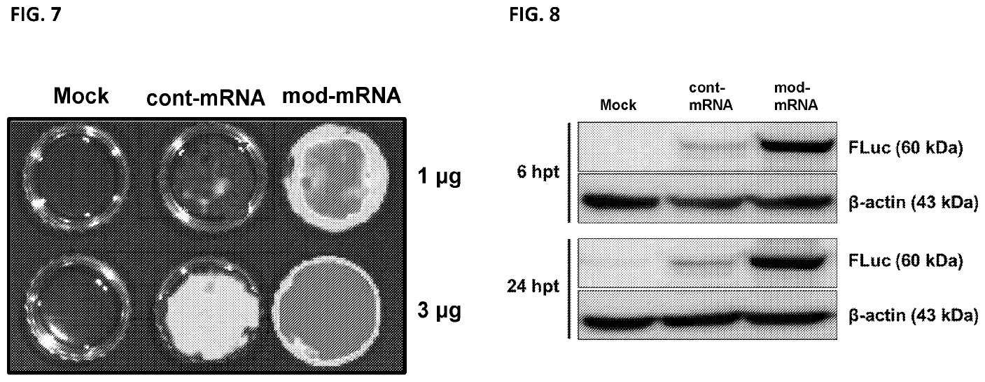

As shown in FIG. 7, compared to cells transfected with the control mRNA construct (middle column), cells transfected with the modified mRNA construct (right column) expressed significantly greater luciferase expression as measured using IVIS imaging system. The increase in luciferase expression appeared to be dependent on dose of the mRNA construct, as much greater luciferase expression was observed at 3 μg in both cells transfected with the modified mRNA construct and cells transfected with the control mRNA construct. Similar results were observed using western blot (see FIG. 8).

The above results confirm the earlier described data and confirm that the 5′-UTR and 3′-UTR sequences provided herein can be used to increase the expression of both influenza protein and other heterologous proteins (e.g., EGFP and Luc).

### Example 5: In Vivo Analysis of Firefly Luciferase (Luc) Protein Expression

To assess whether the 5′-UTR and 3′-UTR sequences of the present disclosure can also increase protein expression in vivo, 6-week old BALB/c mice (female) were used. Briefly, the mice were treated with either the modified mRNA construct encoding Luc or the control mRNA construct. The mRNA constructs were injected intramuscularly into both hind legs of the animals (5 μg/leg). Non-treated animals were used as control. Then, 5 minutes prior to imaging (at 6 hours, 1 day, 2 days, 3 days, 4 days, 5 days, and 6 days post-treatment), the animals were injected (intraperitoneally) with VIVOGLO™ Luciferin (Promega) (3 mg), and the luciferase signal assessed using whole body imaging.

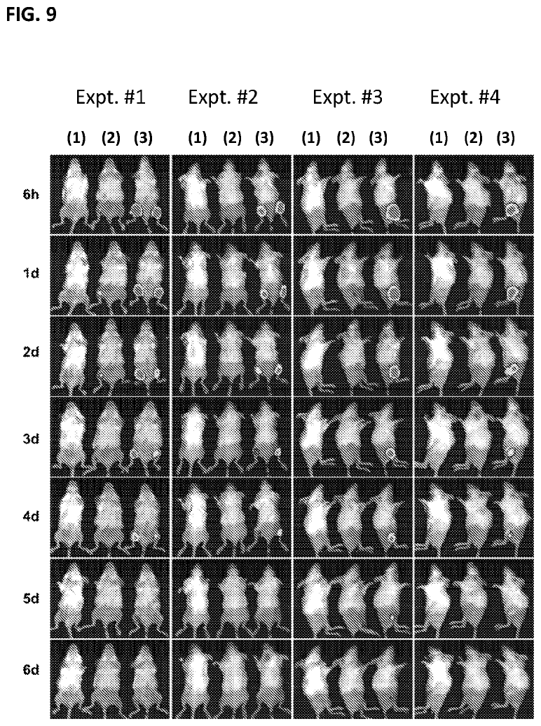

As shown in FIG. 9, some of the animals injected with the control mRNA construct (i.e., Group 2) showed weak luciferase signal at 6 hours post-transfection, which disappeared by about day 3 post-transfection. In contrast, all the animals treated with the modified mRNA construct (i.e., Group 3) exhibited much greater luciferase signal at the site of injection at 6 hours post-transfection. The increased expression was maintained as late as day 5 post-transfection.

These results confirm that the 5′-UTR and 3′-UTR sequences can also increase the expression of protein (including heterologous proteins) in vivo.

### Example 6: Comparison to Control UTR Sequences

To demonstrate the superiority of the 5′-UTR and 3′-UTR sequences provided herein, HEK293T cells were transfected with either the modified EGFP mRNA construct described in Example 3 or with an EGFP mRNA construct comprising a reference UTR sequence (ctcgagagctcgctttcttgctgtccaatttctattaaaggttcctttgttccctaagtccaactactaaactgggggatattatgaagggcc ttgagcatctggattctgcctaataaaaaacatttattttcattgctgcgtcgagagctcgctttcttgctgtccaatttctattaaaggttccttt gttccctaagtccaactactaaactgggggatattatgaagggccttgagcatctggattctgcctaataaaaaacatttattttcattgctg cgtc) (SEQ ID NO: 15). See U.S. Pat. No. 10,301,368 B2. The mRNA constructs were transfected at one of the following doses: 0 μg, 0.5 μg, 1 μg, and 3 μg. Then, at various time points post-transfection (6 hours, 12 hours, and 24 hours), EGFP expression was assessed using both fluorescence microscopy and western blot.

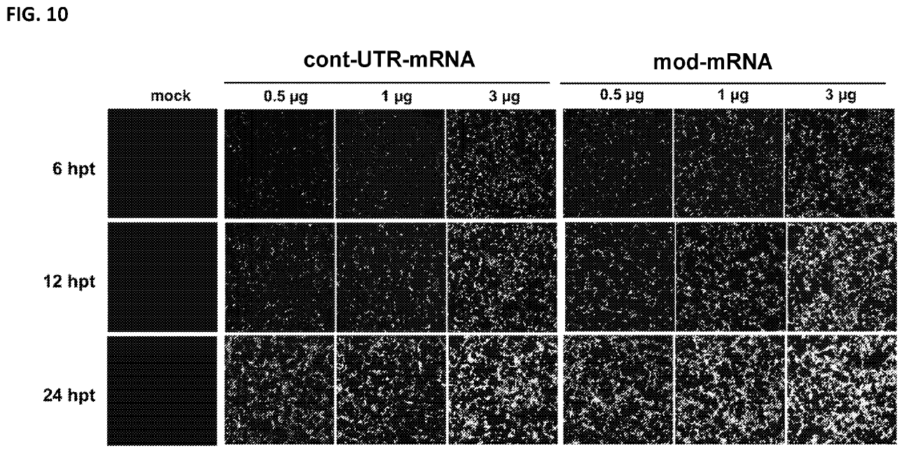

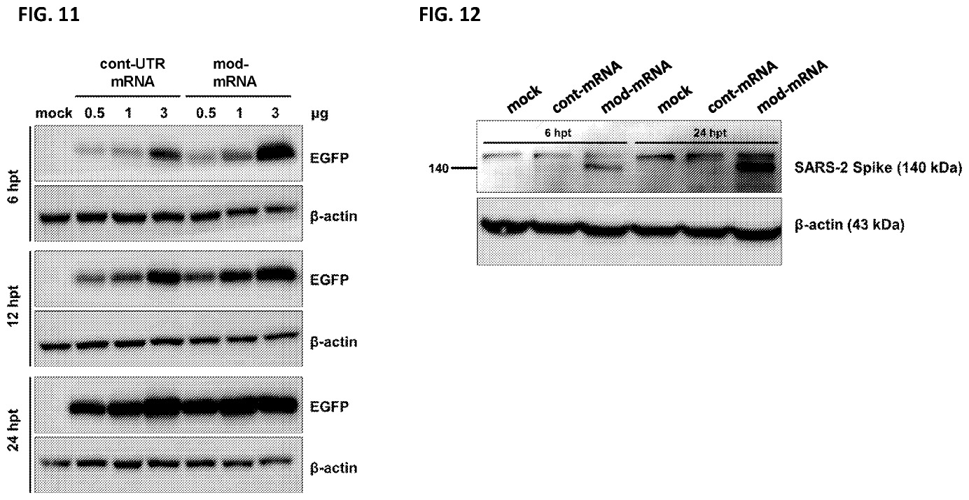

As shown in FIGS. 10 and 11, compared to the control UTR sequences, the 5′-UTR and 3′-UTR sequences provided herein were able to induce greater translation of the EGFP protein as early as 6 hours post-transfection. The increased luciferase expression was apparent at all time points measured, and appeared to be dose dependent (e.g., at 12 hours post-transfection, greater luciferase expression was observed in cells transfected with 3 μg of the modified mRNA construct compared to cells transfected with 0.5 μg of the modified mRNA construct).

The above results suggest that the inclusion of the 5′-UTR and 3′-UTR sequences provided herein can increase translation efficiency to a greater extent compared to other UTR sequences known in the art.

### Example 7: Analysis of Protein Expression of Modified mRNA Construct Encoding a SARS-CoV-2 Spike Protein

Further to the data provided above, whether the 5′-UTR and 3′-UTR sequences provided herein can also increase the expression of other viral proteins, mRNA construct encoding the SARS-CoV-2 spike protein and comprising the 5′-UTR and 3′-UTR sequences described in Example was constructed using the methods provided herein (e.g., Example 1). As a control, a SARS-CoV-2 spike protein mRNA construct lacking the 5′-UTR and 3-UTR sequences was also generated. Then, HEK293 T cells were transfected with the mRNA constructs (3 μg) and the expression of the spike protein was assessed using Western blot.

Similar to the results described herein, cells transfected with the modified SAR-CoV-2 spike protein mRNA construct expressed significantly greater amount of the spike protein compared to cells transfected with the control mRNA construct (see FIG. 12). The results provided here further confirm the ability of the UTR sequences provided herein to increase the expression of a broad array of proteins.

### Example 8: Analysis of the Therapeutic Efficacy of Modified HA mRNA Construct

To assess whether the above-described increase in protein expression correlate with improved therapeutic efficacy, the modified HA mRNA construct described in Examples 1 and 2 (i.e., comprising the 5′-UTR and 3′-UTR sequences described in Example 1) was administered to 6-week-old DBA2/J female mice (OrientBio). The modified mRNA construct was injected intramuscularly to both hind legs (20 μg/leg for a total of 40 μg/mouse). In some of the animals, the modified mRNA construct was formulated in a delivery reagent, i.e., IN VIVO-JETRNA® (Polyplus, France) and then administered to the animals (2.5 μg/leg). Control animals were untreated and not infected (“Mock”) or treated with nuclease free water (Enzynomics). The animals were treated both on days 0 and 28. Then, at day 56 post initial treatment, the animals were infected with the influenza virus (MLD₅₀ of A/South Korea/01/2009). The “mock” control animals were not infected. Both body weight and survival of the animals were monitored for 14 days after infection.

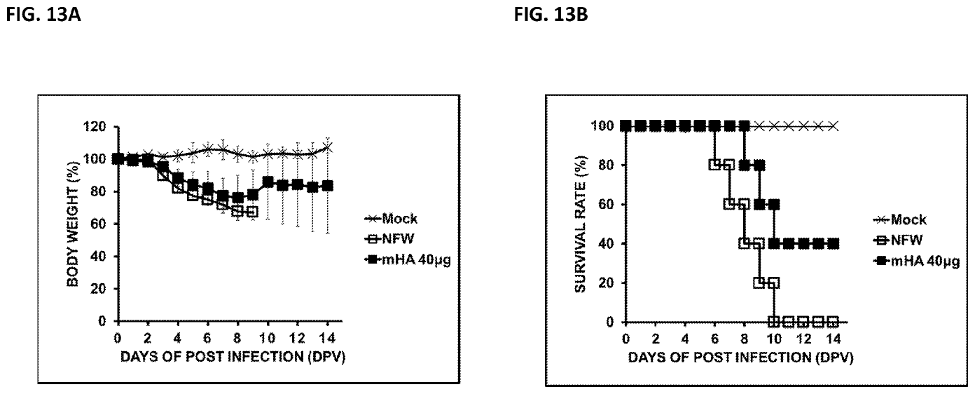

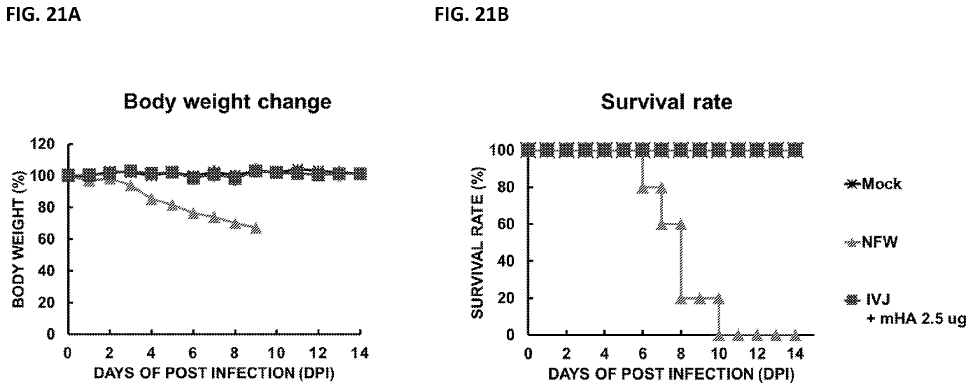

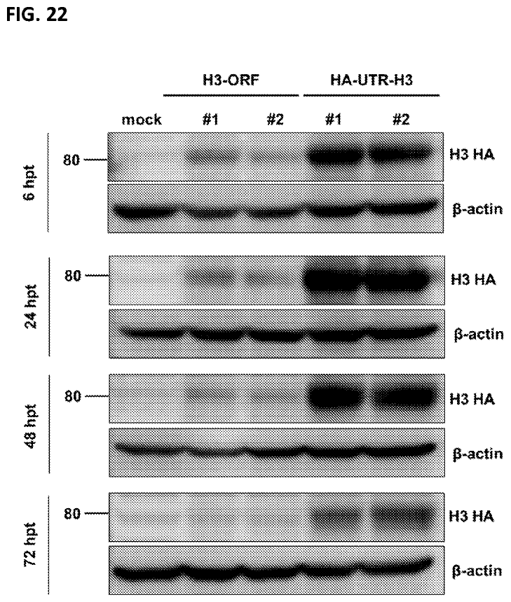

In terms of body weight, no significant differences were observed among animals treated with the modified HA mRNA construct or nuclease free water (see FIG. 13A). However, by day 10 post-infection, all the animals treated with the nuclease free water had succumbed to the infection (see FIG. 13B and FIG. 21B). In contrast, at least 40% of the animals treated with the modified HA mRNA construct were still alive at day 14 post-infection. In animals that were treated with modified HA mRNA formulated in the delivery reagent, all the animals were alive at day 14 post-infection (see FIG. 21B). Not to be bound by any one theory, in some aspects, the delivery reagent could have allowed for greater delivery of the modified HA mRNA construct.

The results provided here demonstrate that the UTR sequences provided herein can also improve therapeutic efficacy, suggesting their inclusion as part of vaccine constructs. Moreover, the improved efficacy observed here was achieved even without the use of any particular delivery agent. And, as demonstrated above, the use of suitable delivery agents (e.g., those described herein) can further improve the therapeutic efficacy of the modified mRNA constructs of the present disclosure.

### Example 9: Comparison to Human Beta Globin UTR Sequences

To further demonstrate the capabilities of the UTR sequences described herein, HEK293T cells were transfected with mRNA construct encoding a green fluorescent protein (GFP) and comprising either (i) the 5′-UTR and 3′-UTR sequences described in Example 1 (“GFP mRNA with HA UTRs”) or (ii) the 5′- and 3′-UTR sequences of human beta globin (hBg) (“GFP mRNA with hBg UTRs”), which are commonly used in the art to increase mRNA expression rate. The sequences for the the hBg-5′-UTR and hBg-3′-UTR are provided in Table 1 (below).

TABLE 1
hBg UTR Sequences

| | |
|---|---|
| hBg-5′-UTR | cagggcagagccatctattgcttacatttgcttc |
| (SEQ ID NO: | tgacacaactgtgttcactagcaacctcaaacag |
| 33) | acacc |
| hBg-3′-UTR | gctcgctttcttgctgtccaatttctattaaagg |
| (SEQ ID NO: | ttcctttgttccctaagtccaactactaaactgg |
| 34) | gggatattatgaagggccttgagcatctggattc |
| | tgcctaataaaaaacatttattttcattg |

Briefly, as described in the earlier examples, HEK293 T cells were seeded (4×10⁵ cells/well) in a 6-well plate at 37 C in Dulbecco's modified Eagle's medium (DMEM; Welgene Inc.). Then, the cells were transfected with 1 μg either of the GFP-encoding mRNA constructs using Lipofectamine2000 (Invitrogen Life Technologies Inc.). Non-transfected cells were used as control. At 6 hours, 24 hours, 48 hours, and 72 hours, GFP signal was measured using various approaches (fluorescent microscopy (Leica Biosystems), Western blot analysis, and flow cytometry).

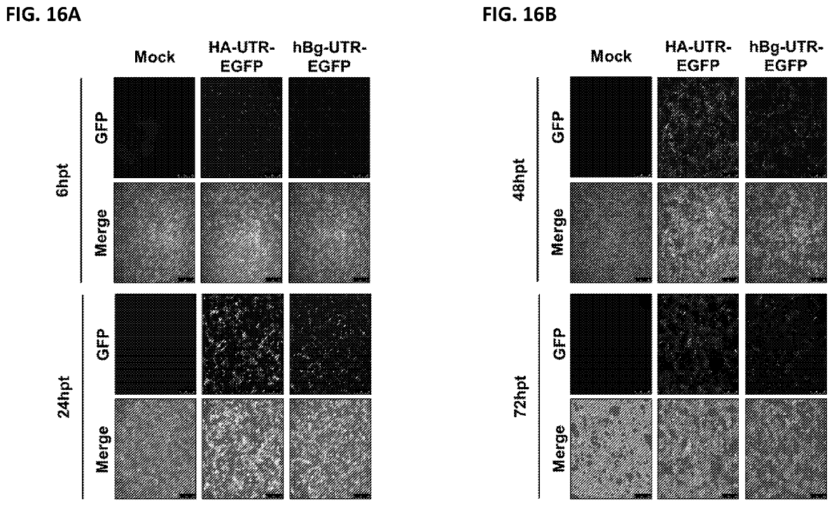

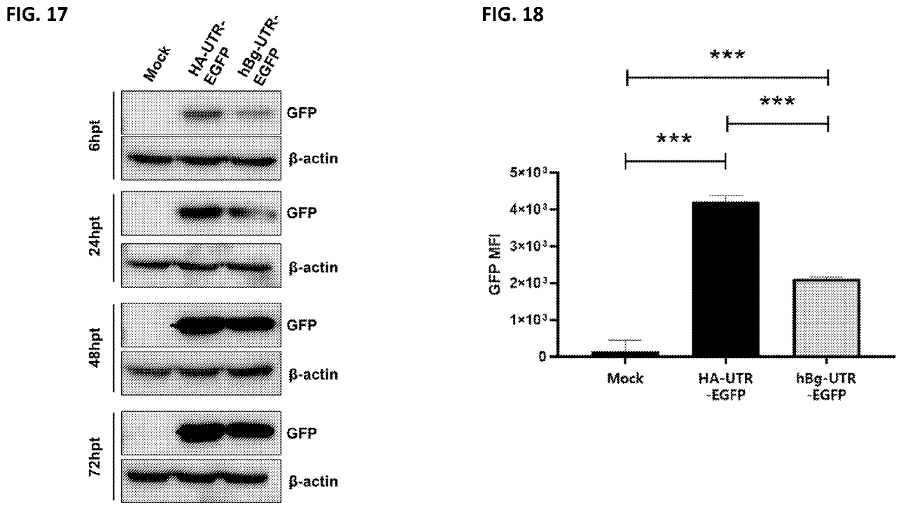

As shown in FIGS. 16A and 16B, for all time points assessed, HEK293 T cells transfected with the GFP mRNA with HA UTRs had consistently higher GFP expression, as compared to those cells transfected with the GFP mRNA with hBg UTRs. Similar results were observed using both Western blot analysis (see FIG. 17) and flow cytometry (see FIG. 18).

To demonstrate that the above-described result is not specific to GFP, luciferase-encoding mRNA constructs comprising either (i) the UTRs of the present disclosure (those described in Example 1) (“Luc mRNA with HA UTRs”) or (ii) the 5′- and 3′-UTR sequences of human beta globin (hBg) (“Luc mRNA with hBg UTRs”) were also constructed and used to transfect the HEK293 T cells. The general transfection strategy was similar to that used above and in the earlier examples (e.g., 1 μg of the mRNA constructs used to transfect with Lipofectamine2000). For bioluminescence analysis, cells were incubated with D-luciferin (150 μg/mL) for 5 minutes in the dark, and then luciferase signal was visualized by IVIS imaging system (PerkinElmer). For Western blot analysis, the general methods were similar to those described in Example 2.

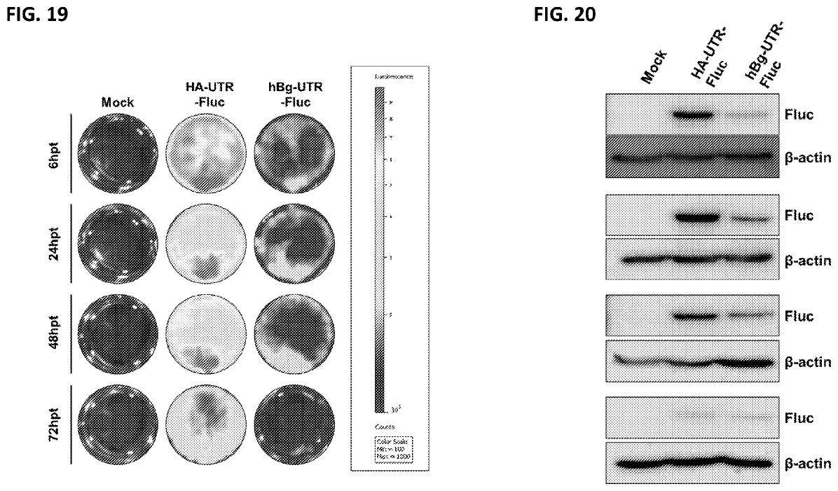

As shown in FIGS. 19 and 20, luciferase expression was also significantly increased in cells transfected with Luc mRNA with HA UTRs, as compared to cells transfected with Luc mRNA with hBg UTRs.

Collectively, the above results further confirm the superiority of the UTRs described herein (i.e., HA-5′-UTR and HA-3′-UTR) in enhancing translation efficiency, compared to other UTRs available in the art.

### Example 10: Further In Vitro Analysis of Influenza HA Protein Expression

Further to Example 2 (provided above), the ability of the UTRs described herein (i.e., 5′-UTR and 3′-UTR sequences described in Example 1) to enhance HA protein expression of different influenza virus strains was assessed. Briefly, using the methods provided in the above examples (e.g., Example 1), mRNA constructs encoding the HA protein from one of the following influenza strains were constructed: (i) A/H3N2, and (ii) B/Yamagata. Some of the mRNA constructs additionally comprised the 5′-UTR (SEQ ID NO: 13) and 3′-UTR (SEQ ID NO: 14) sequences of the hemagglutinin (HA) protein of the 2009 pandemic influenza virus (H1N1pdm09_A/Korea/01/09). Then, the mRNA constructs with and without the UTRs described in Example 1 were used to transfect HEK293 T cells. At various time points post-transfection (6 hours, 24 hours, 48 hours, and 72 hours), Western blot analysis was conducted to measure the expression of the HA protein in the transfected cells. The general methods used for the transfection and Western blot analysis were the same as those described in the earlier examples (see, e.g., Example 2).

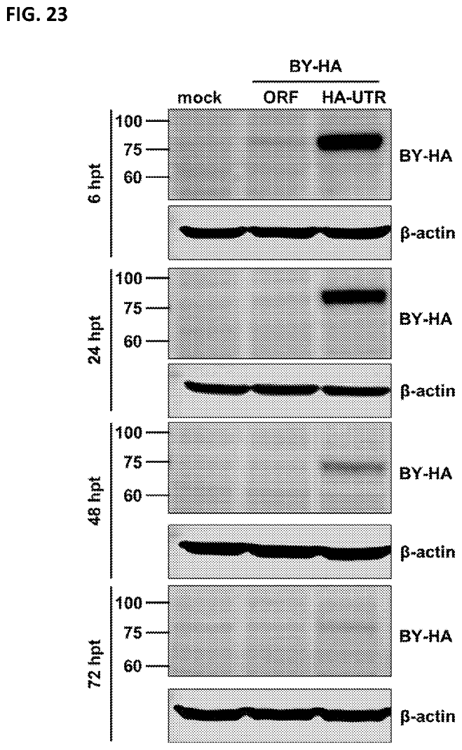

As shown in FIGS. 22 and 23, regardless of the influenza strain, the use of the UTRs described herein increased the expression of the encoded HA protein at all time points assessed. These results demonstrate that the UTRs of the present disclosure (belonging to the 2009 pandemic influenza virus (H1N1pdm09_A/Korea/01/09)) are capable of not only increasing the expression of H1N1 HA protein (see Example 2) but are also useful in increasing expression of HA proteins belonging to other influenza strains.

### Example 11: In Vitro Analysis of Iduronate-2-Sulfatase Expression

Iduronate-2-sulfatase (IDS) is an enzyme involved in the lysosomal degradation of heparin sulfate and dermatan sulfate. Abnormal IDS activity can lead to enzymatic deficiency leading to diseases, such as Hunter syndrome. To assess whether the UTRs described herein (e.g., 5′-UTR and 3′-UTR sequences described in Example 1) could be useful in treating such diseases (e.g., as part of gene replacement therapy), mRNA constructs encoding iduronate-2-sulfatase (IDS) were constructed. Some of the mRNA constructs additionally comprised the 5′-UTR (SEQ ID NO: 13) and 3′-UTR (SEQ ID NO: 14) sequences described herein. Then, the IDS-encoding mRNA constructs with and without the HA-UTRs described herein were used to transfect HEK293 T cells (1 μg or 3 μg). At various time points post-transfection (6 hours, 24 hours, 48 hours, and 72 hours), Western blot analysis was conducted to measure the expression of the HA protein in the transfected cells. The general methods used for the transfection and Western blot analysis were the same as those described in the earlier examples (see, e.g., Example 2).

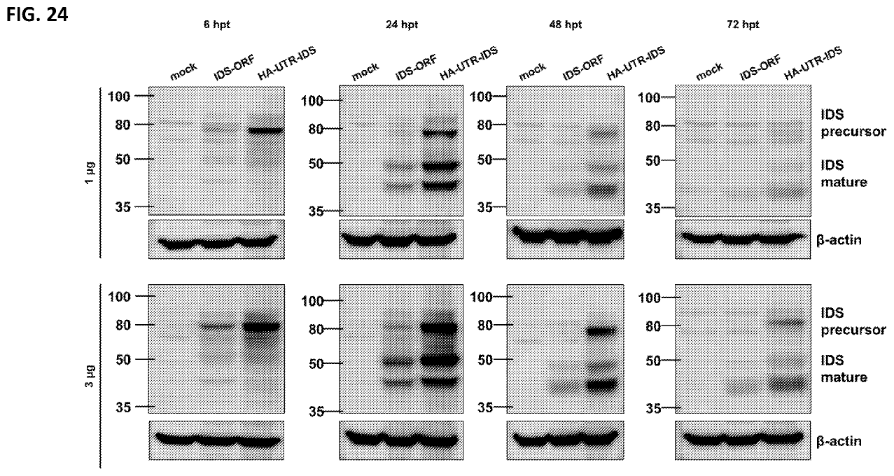

As shown in FIG. 24 (and in agreement with the earlier examples), the inclusion of the UTRs described herein significantly increased the expression of the IDS protein in the transfected cells, as compared to those transfected IDS-encoding mRNA without the UTRs. The increased expression appeared to be dose-dependent, as the highest protein expression was observed in cells transfected with the higher dose of the IDS-encoding mRNA comprising the HA-UTRs described herein.

The above results further demonstrate that the HA-UTRs described herein are capable of increasing the expression of heterologous proteins, including those associated with various genetic diseases. Not to be bound by any one theory, the above results also demonstrate that the HA-UTRs described herein could be useful in developing gene replacement therapies for many genetic diseases.

### Example 12: In Vitro Analysis of Tumor Protein Expression

To further assess the therapeutic potential of the UTRs described herein (e.g., 5′-UTR and 3′-UTR sequences described in Example 1), mRNA constructs encoding certain tumor antigens (NYESO1 or MAGEA3) were constructed. As in the earlier examples, some of the mRNA constructs comprised the 5′-UTR (SEQ ID NO: 13) and 3′-UTR (SEQ ID NO: 14) sequences described herein. Then, the tumor antigen-encoding mRNA constructs with and without the HA-UTRs described herein were used to transfect HEK293 T cells (1 μg), and expression of the tumor antigen was assessed using Western blot. The general methods used for the transfection and Western blot analysis were the same as those described in the earlier examples (see, e.g., Example 2).

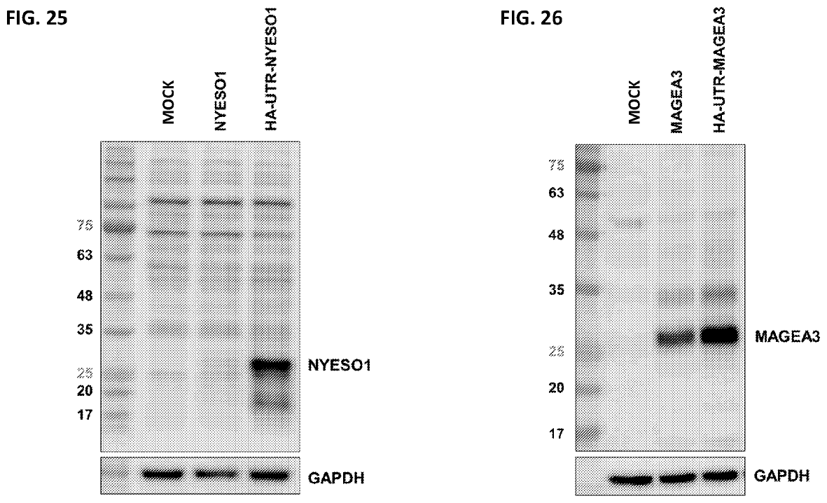

As shown in FIGS. 25 and 26, compared to cells transfected with the mRNA construct lacking the HA-UTRs, cells transfected with the tumor-antigen encoding mRNA construct comprising the HA-UTRs described herein exhibited significantly higher level of tumor antigen expression (both NYESO1 and MAGEA3).

The above results suggest the benefits of the HA-UTRs described herein on cancer immunotherapy, confirming the wide range of therapeutic use associated with the HA-UTRs described herein.

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The contents of all cited references (including literature references, patents, patent applications, and websites) that can be cited throughout this application are hereby expressly incorporated by reference in their entirety for any purpose, as are the references cited therein.

What is claimed is:

1. An isolated polynucleotide comprising an open reading frame (ORF) and (i) a 5′-untranslated region element (5′-UTR) of an influenza hemagglutinin (HA) protein, (ii) a 3′-untranslated region element (3′-UTR) of an influenza hemagglutinin (HA) protein, or both (i) and (ii); wherein the ORF encodes a protein that is heterologous to the 5′-UTR, 3′-UTR, or both 5′-UTR and 3′-UTR.

2. The isolated polynucleotide of claim 1, comprising both the 5′-UTR and the 3′-UTR.

3. The isolated polynucleotide of claim 1 or 2, wherein the 5′-UTR comprises the nucleic acid sequence set forth in SEQ ID NO: 13 (AGCAAAAGCAGGGGAAAATAAAAGCAACAAAA).

4. The isolated polynucleotide of any one of claims 1 to 3, wherein the 5′-UTR consists of the nucleic acid sequence set forth in SEQ ID NO: 13 (AGCAAAAGCAGGGGAAAATAAAAGCAACAAAA).

5. The isolated polynucleotide of any one of claims 1 to 5, wherein the 3′-UTR comprises the nucleic acid sequence set forth in SEQ ID NO: 14 (CATTAGGATTTCAGAAGCATGAGAAAAACACCCTTGTTTCTACT).

6. The isolated polynucleotide of any one of claims 1 to 5, wherein the 3′-UTR consists of the nucleic acid sequence set forth in SEQ ID NO: 14 (CATTAGGATTTCAGAAGCATGAGAAAAACACCCTTGTTTCTACT).

7. The isolated polynucleotide of any one of claims 1 to 6, wherein the 5′-UTR, the 3′-UTR, or both the 5′-UTR and the 3′-UTR are capable of increasing the expression of the heterologous protein encoded by the ORF when transfected in a cell, compared to a corresponding expression in a cell transfected with a reference polynucleotide that does not comprise both the 5′-UTR and the 3′-UTR.

8. The isolated polynucleotide of any one of claims 1 to 7, which further comprises a 5′-cap, a poly(A) tail, at least one translation enhancer element (TEE), a translation initiation sequence, at least one microRNA binding site or seed thereof, a 3′ tailing region of linked nucleosides, an AU rich element (ARE), a post transcription control modulator, or combinations thereof.

9. The isolated polynucleotide of claim 8, wherein the 5′-cap comprises m₂^{7,2′-O}GppₛpGRNA, m⁷GpppG, m⁷Gppppm⁷G, m₂^{(7,3′-O)}GpppG, m₂^{(7,2′-O}GppspG(D1), m₂^{(7,2′-O)}GppspG(D2), m₂^{7,3′-O}Gppp(m₁^{2′-O})ApG, (m⁷G-3′ mppp-G; which may equivalently be designated 3′ O-Me-m7G(5′)ppp(5′)G, N7,2′-O-dimethyl-guanosine-5′-triphosphate-5′-guanosine, m⁷Gm-ppp-G, N7-4-chlorophenoxyethyl)-G(5′)ppp(5′)G, N7-(4-chlorophenoxyethyl)-m^{3′-O}G(5′)ppp(5′)G, 7mG(5′)ppp(5′)N,pN2p, 7mG(5′)ppp(5′)NlmpNp, 7mG(5′)-ppp(5′)NlmpN2 mp, m(7)Gpppm(3)(6,6,2′)Apm(2′)Apm(2′)Cpm(2)(3,2′)Up, inosine, N1-methyl-guanosine, 2′ fluoro-guanosine, 7-deaza-guanosine, 8-oxo-guanosine, 2-amino-guanosine, LNA-guanosine, 2-azido-guanosine, N1-methylpseudouridine, m7G(5′)ppp(5′)(2′OMeA)pG, or combinations thereof.

10. The isolated nanoparticle of claim 8 or 9, wherein the 3′ tailing region of linked nucleosides comprises a poly-A tail, a polyA-G quartet, or a stem loop sequence.

11. The isolated polynucleotide of any one of claims 1 to 8, which comprises at least one modified or non-naturally occurring nucleotide.

12. The isolated polynucleotide of claim 11, wherein the least one modified or non-naturally occurring nucleotide comprises 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, pseudo-uridine, inosine, α-thio-guanosine, 8-oxo-guanosine, 06-methyl-guanosine, 7-deaza-guanosine, N1-methyl adenosine, 2-amino-6-chloro-purine, N6-methyl-2-amino-purine, 6-chloro-purine, N6-methyl-adenosine, α-thio-adenosine, 8-azido-adenosine, 7-deaza-adenosine, pyrrolo-cytidine, 5-methyl-cytidine, N4-acetyl-cytidine, 5-methyl-uridine, 5-iodo-cytidine, or combinations thereof.

13. The isolated polynucleotide of any one of claims 1 to 12, wherein the heterologous protein encoded by the ORF comprises a coronavirus protein.

14. The isolated polynucleotide of claim 13, wherein the coronavirus protein comprises a SARS-CoV-2 spike protein.

15. The isolated polynucleotide of any one of claims 3 to 12, wherein the heterologous protein encoded by the ORF comprises an influenza protein.

16. The isolated polynucleotide of claim 15, wherein the influenza protein comprises a HA protein, a neuraminidase (NA) protein, a nucleoprotein (NP), a matrix 1 (M1) protein, a matrix 2 (M2) protein, a non-structural protein 1 (NS1), a non-structural protein 2 (NS2), a polymerase acidic (PA) protein, a polymerase basic 1 (PB1) protein, a PB1-F2 protein, a polymerase basic 2 (PB2) protein, or any combination thereof.

17. The isolated polynucleotide of any one of claims 1 to 12, wherein the heterologous protein encoded by the ORF comprises a tumor antigen.

18. The isolated polynucleotide of claim 17, wherein the tumor antigen comprises an alpha-fetoprotein (AFP), B-cell maturation antigen (BCMA), carcinoembryonic antigen (CEA), epithelial tumor antigen (ETA), mucin 1 (MUC1), Tn-MUC1, mucin 16 (MUC16), tyrosinase, melanoma-associated antigen (MAGE; e.g., MAGEA3), tumor protein p53 (p53), CD4, CD8, CD45, CD80, CD86, programmed death ligand 1 (PD-L1), programmed death ligand 2 (PD-L2), prostate-specific membrane antigen (PSMA), TAG-72, human epidermal growth factor receptor 2 (HER2), GD2, cMET, EGFR, mesothelin, VEGFR, alpha-folate receptor, CE7R, IL-3, cancer-testis antigen (e.g., New York esophageal squamous cell carcinoma 1 (NY-ESO-1), MART-1 gp100, ROR1, ROR2, glypican-2, glypican-3, TNF-related apoptosis-inducing ligand, or combinations thereof.

19. The isolated polynucleotide of any one of claims 1 to 12, wherein the heterologous protein encoded by the ORF comprises a protein associated with a genetic disorder.

20. The isolated polynucleotide of claim 19, wherein the genetic disorder comprises a Hunter syndrome.

21. An isolated polynucleotide comprising, from 5′ to 3′:
(a) a 5′-untranslated region element (5′-UTR) of an influenza hemagglutinin (HA) protein, which comprises the nucleic acid sequence set forth in SEQ ID NO: 13 (AGCAAAAGCAGGGGAAAATAAAAGCAACAAAA);
(b) an open reading frame (ORF); and
(c) a 3′-untranslated region element (3′-UTR) of an influenza hemagglutinin (HA) protein, which comprises the nucleic acid sequence set forth in SEQ ID NO: 14 (CATTAGGATTTCAGAAGCATGAGAAAAACACCCTTGTTTCTACT);
wherein the ORF encodes a protein that is heterologous to both the 5′-UTR and the 3′-UTR.

22. A vector comprising the isolated polynucleotide of any one of claims 1 to 21.

23. A cell comprising the isolated polynucleotide of any one of claims 1 to 21 or the vector of claim 22.

24. A pharmaceutical composition comprising (i) the isolated polynucleotide of any one of claims 1 to 21, the vector of claim 22, or the cell of claim 23; and (ii) a pharmaceutically acceptable excipient.

25. A kit comprising (i) the isolated polynucleotide of any one of claims 1 to 21, the vector of claim 22, or the cell of claim 23; and (ii) instructions for use.

26. A method of treating a disease or disorder in a subject in need thereof, comprising administering to the subject the isolated polynucleotide of any one of claims 1 to 21.

27. The method of claim 26, wherein the disease or disorder comprises a viral infection, cancer, genetic disorder, or combinations thereof.

28. The method of claim 27, wherein the viral infection comprises a coronavirus infection, influenza virus infection, or both.

29. The method of claim 27, wherein the cancer comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or combinations thereof.

30. The method of claim 27, wherein the genetic disorder comprises a Hunter syndrome.

31. A method of inducing an immune response in a subject in need thereof, comprising administering to the subject the isolated polynucleotide of any one of claims 1 to 21, wherein after the administration, an immune response against the heterologous protein encoded by the ORF is induced in the subject.

32. A method of increasing the expression of a protein, comprising contacting a cell with the isolated polynucleotide of any one of claims 1 to 21.

33. The method of claim 32, wherein the contacting occurs in vivo.

34. The method of claim 32, wherein the contacting occurs ex vivo.

35. The method of any one of claims 32 to 34, wherein the expression of the protein is increased by at least about 0.5-fold, about 1-fold, about 2-fold, about 3-fold, about 4-fold, about 5-fold, about 10-fold, about 20-fold, about 30-fold, about 40-fold, or about 50-fold, compared to the expression of the protein in a cell contacted with a reference polynucleotide that does not comprise both the 5′-UTR and the 3′-UTR.

36. The method of any one of claims 26 to 35, wherein the isolated polynucleotide is delivered in a delivery agent.

37. The method of claim 36, wherein the delivery agent comprises a micelle, an exosome, a lipidoid, a liposome, a lipoplex, a lipid nanoparticle, an extracellular vesicle, a synthetic vesicle, a polymeric compound, a peptide, a protein, a cell, a nanoparticle mimic, a nanotube, a conjugate, a viral vector, or combinations thereof.

38. The method of claim 36 or 37, wherein the delivery agent comprises a cationic carrier unit comprising:
[WP]-L1-[CC]-L2-[AM] (formula I)
or
[WP]-L1-[AM]-L2-[CC] (formula II),
wherein
WP is a water-soluble biopolymer moiety;
CC is a cationic carrier moiety;
AM is an adjuvant moiety; and,
L1 and L2 are independently optional linkers.

39. The method of claim 38, wherein the cationic carrier unit and the isolated polynucleotide are capable of associating with each other to form a micelle when mixed together.

40. The method of claim 39, wherein the association is via a covalent bond.

41. The method of claim 39, wherein the association is via a non-covalent bond.

42. The method of claim 41, wherein the non-covalent bond comprises an ionic bond.

43. The method of any one of claims 38 to 42, wherein the water-soluble polymer comprises poly(alkylene glycols), poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyglycerol, polyphosphazene, polyoxazolines (“POZ”) poly(N-acryloylmorpholine), or any combinations thereof.

44. The method of any one of claims 38 to 43, wherein the water-soluble polymer comprises polyethylene glycol (“PEG”), polyglycerol, or poly(propylene glycol) (“PPG”).

45. The method of any one of claims 38 to 44, wherein the water-soluble polymer comprises: 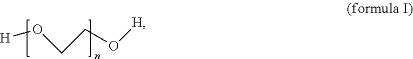
wherein n is 1-1000.

46. The method of claim 45, wherein the n is at least about 110, at least about 111, at least about 112, at least about 113, at least about 114, at least about 115, at least about 116, at least about 117, at least about 118, at least about 119, at least about 120, at least about 121, at least about 122, at least about 123, at least about 124, at least about 125, at least about 126, at least about 127, at least about 128, at least about 129, at least about 130, at least about 131, at least about 132, at least about 133, at least about 134, at least about 135, at least about 136, at least about 137, at least about 138, at least about 139, at least about 140, or at least about 141.

47. The method of claim 45, wherein the n is about 80 to about 90, about 90 to about 100, about 100 to about 110, about 110 to about 120, about 120 to about 130, about 140 to about 150, or about 150 to about 160.

48. The method of any one of claims 45 to 47, wherein the n is about 114.

49. The method of any one of claims 38 to 48, wherein the water-soluble polymer is linear, branched, or dendritic.

50. The method of any one of claims 38 to 49, wherein the cationic carrier moiety comprises one or more basic amino acids.

51. The method of claim 50, wherein the cationic carrier moiety comprises at least about three, at least about four, at least about five, at least about six, at least about seven, at least about eight, at least about nine, at least about ten, at least about 11, at least about 12, at least about 13, at least about 14, at last about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, or at least about 50 basic amino acids.

52. The method of claim 51, wherein the cationic carrier moiety comprises about 60, about 70, about 80, about 90, or about 100 basic amino acids.

53. The method of claim 52, wherein the cationic carrier moiety comprises about 80 basic amino acids.

54. The method of any one of claims 50 to 53, wherein the basic amino acid comprises arginine, lysine, histidine, or any combination thereof.

55. The method of any one of claims 38 to 54, wherein the cationic carrier moiety comprises about 80 lysine monomers.

56. The method of any one of claims 38 to 55, wherein the adjuvant moiety is capable of modulating an immune response, an inflammatory response, and/or a tissue microenvironment.

57. The method of any one of claims 38 to 56, wherein the adjuvant moiety comprises an imidazole derivative, an amino acid, a vitamin, or any combination thereof.

58. The method of claim 57, wherein the adjuvant moiety comprises: 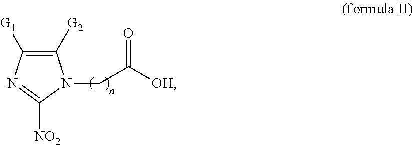
wherein each of G1 and G2 is H, an aromatic ring, or 1-10 alkyl, or G1 and G2 together form an aromatic ring, and wherein n is 1-10.

59. The method of claim 57, wherein the adjuvant moiety comprises nitroimidazole.

60. The method of claim 57, wherein the adjuvant moiety comprises metronidazole, tinidazole, nimorazole, dimetridazole, pretomanid, ornidazole, megazol, azanidazole, benznidazole, or any combination thereof.

61. The method of any one of claims 38 to 60, wherein the adjuvant moiety comprises an amino acid.

62. The method of claim 61, wherein the adjuvant moiety comprises 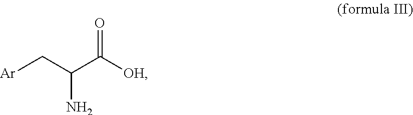
wherein Ar is 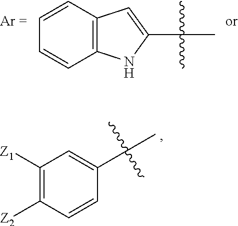
and
wherein each of Z1 and Z2 is H or OH.

63. The method of any one of claims 38 to 62, wherein the adjuvant moiety comprises a vitamin.

64. The method of claim 63, wherein the vitamin comprises a cyclic ring or cyclic hetero atom ring and a carboxyl group or hydroxyl group.

65. The method of claim 63 or 64, wherein the vitamin comprises: 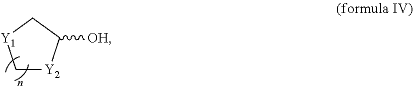
wherein each of Y1 and Y2 is C, N, O, or S, and wherein n is 1 or 2.

66. The method of any one of claims 63 to 65, wherein the vitamin is selected from the group consisting of vitamin A, vitamin B1, vitamin B2, vitamin B3, vitamin B6, vitamin B7, vitamin B9, vitamin B12, vitamin C, vitamin D2, vitamin D3, vitamin E, vitamin M, vitamin H, and any combination thereof.

67. The method of claim 66, wherein the vitamin is vitamin B3.

68. The method of claim 66 or 67, wherein the adjuvant moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, or at least about 20 vitamin B3.

69. The method of any one of claims 66 or 68, wherein the adjuvant moiety comprises about 5 vitamin B3.

70. The method of any one of claims 66 to 69, wherein the delivery agent comprises a water-soluble biopolymer moiety with about 120 to about 130 PEG units, a cationic carrier moiety comprising a poly-lysine with about 80 lysines, and an adjuvant moiety with about 5 vitamin B3.

71. The method of claim 36 or 37, wherein the delivery agent comprises a cationic carrier unit comprising:
[CC]-L1-[CM]-L2-[HM] (Schema I);
[CC]-L1-[HM]-L2-[CM] (Schema II);
[HM]-L1-[CM]-L2-[CC] (Schema III);
[HM]-L1-[CC]-L2-[CM] (Schema IV);
[CM]-L1-[CC]-L2-[HM] (Schema V); or
[CM]-L1-[HM]-L2-[CC] (Schema VI);
wherein
CC is a positively charged carrier moiety;
CM is a crosslinking moiety;
HM is a hydrophobic moiety; and,
L1 and L2 are independently optional linkers, and
wherein the number of HM is less than 40% relative to [CC] and [CM].

72. The method of claim 71, wherein the number of HM is less than 39%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or about 1% relative to [CC] and [CM].

73. The method of claim 71 or 72, the cationic carrier unit is capable of interacting with the isolated polynucleotide according to claims 1-21.

74. The method of claims 71 to 73, wherein the cationic carrier moiety comprises at least about three, at least about four, at least about five, at least about six, at least about seven, at least about eight, at least about nine, at least about ten, at least about 11, at least about 12, at least about 13, at least about 14, at least 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, at least about 35, at least about 36, at least about 37, at least about 38, at least about 39, at least about 40, at least about 41, at least about 42, at least about 43, at least about 44, at least about 45, at least about 46, at least about 47, at least about 48, at least about 49, at least about 50, at least about 51, at least about 52, at least about 53, at least about 54, at least about 55, at least about 56, at least about 57, at least about 58, at least about 59, at least about 60, at least about 61, at least about 62, at least about 63, at least about 64, at least about 65, at least about 66, at least about 67, at least about 68, at least about 69, at least about 70, at least about 71, at least about 72, at least about 73, at least about 74, at least about 75, at least about 76, at least about 77, at least about 78, at least about 79, or at least about 80 amino acids.

75. The method of claim 74, wherein the cationic carrier moiety comprises about 80 amino acids.

76. The method of claim 75, wherein the amino acids comprise lysines.

77. The method of claims any one of claims 71 to 76, wherein the hydrophobic moiety comprises at least about two, at least about three, at least about four, at least about five, at least about six, at least about seven, at least about eight, at least about nine, at least about ten, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 26, at least about 27, at least about 28, at least about 29, at least about 30, at least about 31, at least about 32, at least about 33, at least about 34, or at least about 35 amino acids, each linked to a vitamin.

78. The method of claim 77, wherein the hydrophobic moiety comprises about two vitamin B3, about three vitamin B3, about four vitamin B3, about five vitamin B3, about six vitamin B3, about seven vitamin B3, about eight vitamin B3, about nine vitamin B3, about ten vitamin B3, about 11 vitamin B3, about 12 vitamin B3, about 13 vitamin B3, about 14 vitamin B3, about 15 vitamin B3, about 16 vitamin B3, about 17 vitamin B3, about 18 vitamin B3, about 19 vitamin B3, about 20 vitamin B3, about 21 vitamin B3, about 22 vitamin B3, about 23 vitamin B3, about 24 vitamin B3, about 25 vitamin B3, about 26 vitamin B3, about 27 vitamin B3, about 28 vitamin B3, about 29 vitamin B3, about vitamin B3, about 31 vitamin B3, about 32 vitamin B3, about 33 vitamin B3, about 34 vitamin B3, or about 35 vitamin B3.

79. The method of any one of claims 71 to 78, wherein the cationic carrier moiety comprises about 35 to about 45 lysines, the crosslinking moiety comprises about 5 to about 40 lysine-thiol, and the hydrophobic moiety comprises about 1 to about 10 lysine-vitamin B3.

80. The method of claim 79, wherein the cationic carrier moiety comprises about 40 lysines, the crosslinking moiety comprises about 35 lysine-thiol, and the hydrophobic moiety comprises about 5 lysine-vitamin B3.

81. The method of any one of claims 71 to 80, wherein the water-soluble biopolymer moiety comprises about 120 to about 130 PEG units.

82. The method of claim 81, wherein the water-soluble biopolymer moiety comprises about 114 PEG units.

83. The method of any one of claims 26 to 81, wherein the isolated polynucleotide is administered parenthetically, intramuscularly, subcutaneously, ophthalmic, intravenously, intraperitoneally, intradermally, intraorbitally, intranasally, intracerebrally, intracranially, intracerebroventricularly, intraspinally, intraventricular, intrathecally, intracistemally, intracapsularly, intratumorally, topically, or any combination thereof.
